(19) United States
(12) Patent Application Publication
(10) Pub. No.: US 2015/0159166 A1
(43) Pub. Date: Jun. 11, 2015

(54) Plant drought tolerance and nitrogen use efficiency by reducing plant sensitivity to ethylene

(71) Applicant: PIONEER HI BRED INTERNATIONAL INC, JOHNSTON, IA (US)

(72) Inventors: Rayeann Archibald, Altoona, IA (US); Wesley Bruce, Raleigh, NC (US); Mei Guo, West Des Moines, IA (US); Rajeev Gupta, Johnston, IA (US); Mary Rupe, Altoona, IA (US); Kathleen Schellin, West Des Monies, IA (US); Jinrui Shi, Johnston, IA (US); Carl Simmons, Des Moines, IA (US)

(21) Appl. No.: 14/355,249

(22) PCT Filed: Oct. 29, 2012
(86) PCT No.: PCT/US2012/062392

§ 371 Date: Apr. 30, 2014

Related U.S. Application Data
(60) Provisional application No. 61/553,443, filed on Oct. 31, 2011.

(51) Int. Cl. C12N 15/82; C07K 14/415; A01H 1/04

(52) U.S. Cl. CPC C12N 15/8249; C12N 15/8271; C12N 15/8273; C12N 15/8261; A01H 1/04; C07K 14/415

(57) ABSTRACT

The present disclosure provides polynucleotides and related polypeptides which are used to modify ethylene sensitivity in plants. Ethylene insensitive transgenic maize plants produce higher grain yields in water deficient and low nitrogen environments than non-transgenic plants. Through controlled expression of the transgene in desired tissues and organs, or specific plant developmental stages, the ethylene perception and signal transduction is altered to create transgenic plants which yield better under abiotic stress.

### FIELD OF THE DISCLOSURE

The disclosure relates generally to the field of molecular biology.

### BACKGROUND

The domestication of many plants has correlated with dramatic increases in yield. Most phenotypic variation occurring in natural populations is continuous and is effected by multiple gene influences. The identification of specific genes responsible for the dramatic differences in yield, in domesticated plants, has become an important focus of agricultural research.

Ethylene (C₂H₄) is a gaseous plant hormone that affects myriad developmental processes and fitness responses in plants, such as germination, flower and leaf senescence, fruit ripening, leaf or fruit abscission, root nodulation, programmed cell death and responsiveness to stress and pathogen attack. Additional ethylene effects include stem extension of aquatic plants, gas space (aerenchyma) development in roots, leaf epinastic curvatures, stem and shoot swelling (in association with stunting), femaleness in curcubits, fruit growth in certain species, apical hook closure in etiolated shoots, root hair formation, flowering in the Bromeliaceae, diageotropism of etiolated shoots and increased gene expression (e.g., of polygalacturonase, cellulase, chitinases, β1,3-glucanases, etc.). These effects are sometimes affected by the action of other plant hormones, other physiological signals and the environment, both biotic and abiotic.

Ethylene is released by ripening fruit and is also produced by most plant tissues, e.g., in response to stress (e.g., drought, crowding, pathogen attack, temperature stress, wounding, etc.) and in maturing and senescing organs. Genetic screens have identified more than a dozen genes involved in the ethylene response in plants.

Ethylene is generated from methionine by a well-defined pathway involving the conversion of S-adenosyl-L-methionine (SAM or Ado Met) to the cyclic amino acid 1-aminocyclopropane-1-carboxylic acid (ACC) which is facilitated by ACC synthase. Ethylene is then produced from the oxidation of ACC through the action of ACC oxidase. Alternatively, ACC may be converted into a-ketobutyric acid and ammonia by the action of ACC deaminase.

The phytohormone ethylene modulates plant growth and development as well as biotic and abiotic stress responses in plants. Experimental activities shown here demonstrate that ectopic expression of ARGOS genes renders the plants insensitive to ethylene. Ethylene insensitive maize plants produce higher grain yields in water deficient and low nitrogen environments than non-transgenic plants having normal sensitivity to ethylene. Through controlled expression of ARGOS transgene in desired tissues and organs, or specific plant developmental stages, the ethylene perception and signal transduction are altered by design to create transgenic plants which yield better under abiotic stress.

### BRIEF SUMMARY

Methods embodied by this disclosure include: a method of modulating the ethylene sensitivity in a plant, comprising: introducing into a plant cell a recombinant construct comprising a polynucleotide encoding a transmembrane protein comprising a proline rich motif having a sequence PPLXPPPX (SEQ ID NO: 96), wherein the proline rich domain is located between a first transmembrane sequence and a second transmembrane sequence, operably linked to a promoter; and expressing said polynucleotide to modulate the level of ethylene sensitivity in said plant, also this same wherein the proline rich motif (PRM) sequence comprises original PRM (SEQ ID NO: 88), or variant PRM (SEQ ID NO: 102).

An addition this method wherein: the plant is selected from the group consisting of: maize, soybean, sorghum, canola, wheat, alfalfa, cotton, rice, barley, millet, peanut, sugarcane, miscanthus, poaceae, cocoa, camelina, Ipomoea and Solanum; the ethylene sensitivity is decreased; said construct is an over expression construct; said construct comprises SEQ ID NO: 88 or SEQ ID NO: 102.

Another embodiment would include method of modulating the ethylene sensitivity in a plant, comprising: introducing into a plant cell a nucleotide construct comprising a polynucleotide which encodes a TPT domain having at least 50% sequence identity to that of TM1 SEQ ID NO: 90 or TM2 SEQ ID NO: 91 operably linked to a promoter, also including the proline motif aforementioned and growing the plant under either a drought or a low nitrogen condition; wherein the plant is: selected from the group consisting of: maize, soybean, sorghum, canola, wheat, alfalfa, cotton, rice, barley, millet, peanut, sugarcane, poaceae, cocoa, camelina, Ipomoea and Solanum, is from a monocot, is from maize.

Embodiments also include plants produced by the aforementioned mentods, including: wherein the plant has decreased ethylene sensitivity when compared to a plant which has not been transformed; wherein the plant has decreased susceptibility to abiotic stress; wherein the plant has decreased susceptibility to drought stress; wherein the plant has decreased susceptibility to crowding stress; wherein the plant has decreased susceptibility to flooding stress.

Additional embodiments include isolated protein comprising: polypeptide of at least 20 contiguous amino acids from a polypeptide of SEQ ID NO: 89; a polypeptide of SEQ ID NO: 89;

a polypeptide having at least 80% sequence identity to, and having at least one linear epitope in common with, a polypeptide of SEQ ID NO: 89, wherein said sequence identity is determined using BLAST 2.0 under default parameters; and, at least one polypeptide as describe in previous embodiments..

Embodiments of the disclosure include: an isolated polynucleotide sequence encoding a protein with ethylene regulatory activity having the sequence of SEQ ID NO: 89 and polypeptide with ethylene regulatory activity having the sequence of SEQ ID NO: 89.

Methods are provided for ectopic expression of ARGOS genes in plants to affect plant sensitivity to ethylene. ZmARGOS constructs demonstrated improved drought tolerance, nitrogen use efficiency and reduced plant sensitivity to ethylene.

Compositions and methods for controlling plant growth for increasing yield under stress in a plant are provided. The compositions include ARGOS sequences from maize, soybean, arabidopsis, rice and sorghum. Compositions of the disclosure comprise amino acid sequences and nucleotide sequences selected from SEQ ID NOS: 1-37, 40-91 and 96 as well as variants and fragments thereof.

Polynucleotides encoding the ARGOS sequences are provided in DNA constructs for expression in a plant of interest. Expression cassettes, plants, plant cells, plant parts and seeds comprising the sequences of the disclosure are further provided. In specific embodiments, the polynucleotide is operably linked to a constitutive promoter.

Methods for modulating the level of an ARGOS sequence in a plant or plant part is provided. The methods comprise introducing into a plant or plant part a heterologous polynucleotide comprising an ARGOS sequence of the disclosure. The level of ARGOS polypeptide can be increased or decreased. Such method can be used to increase the yield in plants; in one embodiment, the method is used to increase grain yield in cereals.

Method of increasing yield in a crop plant, the method includes expressing a recombinant construct comprising a polynucleotide encoding a transmembrane protein comprising a proline rich motif having a sequence PPLXPPPX (SEQ ID NO: 96), wherein the proline rich domain is located between a first transmembrane sequence and a second transmembrane sequence, operably linked to a promoter; and increasing the yield of the crop plant, wherein the yield is increased under lower than normal nitrogen levels. In an embodiment, the lower nitrogen level is about 10% to about 40% less compared to a normal nitrogen level. In an embodiment, the lower nitrogen level is reduced to about 50% less compared to a normal nitrogen level. In an embodiment, the applied nitrogen level is reduced during a later reproductive stage of the plant. In an embodiment, the crop plant is maize and is hybrid maize.

A method of improving an agronomic parameter of a maize plant, the method includes expressing a recombinant construct comprising a polynucleotide encoding a transmembrane protein comprising a proline rich motif having a sequence PPLXPPPX (SEQ ID NO: 96), wherein the proline rich domain is located between a first transmembrane sequence and a second transmembrane sequence, operably linked to a promoter; and improving at least one of the agronomic parameters selected from the group consisting of root growth, shoot biomass, root biomass, kernel number, ear size, and drought stress.

A method of marker-assisted selection of a maize plant that exhibits an altered expression pattern of an endogenous gene, the method includes obtaining a maize plant comprising an allelic variation in the genomic region of a polynucleotide encoding a transmembrane protein comprising a proline rich motif having a sequence PPLXPPPX (SEQ ID NO: 96), wherein the expression of the polynucleotide is increased compared to a control maize plant not having the variation; selecting the maize plant comprising the variation; and developing a population of maize plants comprising the variation through marker-assisted selection process. In an embodiment, the variation is present in the regulatory region of the genomic region. In an embodiment, the variation is present in the coding region of the polynucleotide. In an embodiment, the variation is present in the non-coding region of the genomic region. In an embodiment, the expression of the polynucleotide is increased differentially in different genetic backgrounds.

### BRIEF DESCRIPTION OF THE DRAWINGS

FIG. 1: Dendrogram illustrating the relationship between the ARGOS polypeptides of this disclosure from various plant species: maize, rice, soybean, sorghum and arabidopsis.

FIG. 2: Alignment of the maize, rice, soybean, sorghum and arabidopsis polypeptide sequences with identification of conserved regions. The proteins have a well-conserved proline-rich region near the C-terminus. The N-termini are generally diverged. The proteins are quite short, ranging from 58 to 146, and averaging 110 amino acids.

FIG. 3: Alignment of ZmARGOS1, 2 and 3, with AtARGOS1 and 4, highlighting their areas of consensus and conservative substitutions.

FIG. 4. ARGOS8 transformation into an inbred. Data collected from a T1 inbred field observation. (A) representative ears, (C) ear length, (B) plant height, (D) stalk diameter measurements.

FIG. 5. Sequence alignment of ZmARGOS1 (SEQ ID NO: 2) vs. ZmARGOS8 (SEQ ID NO: 44).

FIG. 6. Predicted protein structure of ZmARGOS1 and ZmARGOS8. FIG. 7. Effect of ZmARGOS8 on plant biomass accumulation at seedling stage under 3 nitrogen concentrations. * indicated a statistical significant difference from non-transgenic null at p<0.05.

FIG. 8. Field grain yield of transgenic ZmARGOS8 in multiple location tests. Events with * showed a statistical significant difference from non-transgenic null at p<0.1.

FIG. 9. Effect of ZmARGOS8 on plant and ear growth under 2 mM nitrate concentrations. * indicted a statistical significant difference from non-transgenic null at p<0.05.

FIG. 10. Effect of ZmARGOS8 on plant and ear growth under 6.5 mM nitrate concentrations. * indicted a statistical significant difference from non-transgenic null at p<0.05.

FIG. 11. Effects of ZmARGOS1 overexpression on ethylene biosynthesis and responses in maize plants, structure of TPT domain-containing transmembrane ARGOS proteins and hormonal regulation of ARGOS gene expression in maize.

(A) Increased ethylene production in Ubi:ZmARGOS1 maize transgenic plants. The two uppermost collared leaves of V7 plants of inbred PHWWE were analyzed. Ethylene was collected for a period of 20 hr and subsequently measured using a gas chromatograph. Ethylene production in transgenic plants (TR) and wild-type segregants (WT) was calculated based on tissue fresh weight. Mean±standard deviation were determined for six replications. Three transgenic events (E1, E2 and E3) are shown. (B) Five-day-old maize seedlings of ZmARGOS1 transgenic plants (TR) and wild-type segregants (WT) germinated in the dark in the presence of 0 (upper), 25 (middle) or 100 μM (bottom) of the ethylene precursor ACC. One representative event is shown.

(C) Schematic presentation of structure of maize ARGOS proteins and Arabidopsis homologs. The TPT domain in maize ZmARGOS1 consists of two predicted transmembrane helices (TM1, aa79-101; TM2, aa110-134) and the proline-rich motif (PRM, aa102PPLPPPPS109) (upper). Predicted orientation of the transmembrane helices (TM1 and TM2), the connecting loop (proline-rich motif, PRM), and the N- and C-terminal sequences in membranes is shown in lower panel.

(D) Induction of ZmARGOS1 and ZmARGOS8 gene expression by hormonal treatment. Maize V3 seedlings were sprayed with 50 uM ACC, 50 uM ABA, 20 uM cytokinin (N-6-benzylaminopurine), 100 uM jasmonic acid (JA), and 10 uM IAA. Leaf tissues were harvested 2 and 4 hr for RNA extraction. The gel stained with ethidium bromide is shown as a control for loading.

FIG. 12. Sequence alignment of the ARGOS genes to show the conserved region among the family members and homologs across grass species. Conserved region is identified as LX1X2LPLX3LPPLX4X5PP (SEQ ID NO: 86) where X1=L,V,I; X2=L,V,I,F; X3=V,L,A; X4=P,Q,S; X5=P,A.

FIG. 13. Overexpression of ZmARGOS1 conferring ethylene insensitivity in Arabidopsis

(A) Comparison of 3-day-old dark-grown seedlings germinated in the presence or absence of the ethylene precursor ACC (10 μM). Representative seedlings of wild-type Col-0 (WT), vector controls and ZmARGOS1 transgenic plants are shown. (B) Comparison of 3-day-old etiolated seedlings germinated in the presence of 10, 50 or 100 ppm gaseous ethylene.

(C) ZmARGOS1 transgenic plants (right) and vector controls (left) grown in a growth chamber at 24° C. in the light (16 hr of illumination at an intensity of approximately 120 mE m⁻² s⁻¹) and 23° C. in the dark (8 hr).

Upper panel, 16-days after planting (DAP) plants showing smaller rosette in transgenic plants; bottom, 39-DAP plants showing delayed flowering and leaf senescence phenotypes.

(D) Inflorescences of ZmARGOS1 transgenic (upper right) and vector control plants (upper left) grown under the same conditions as in (A). Transgenic plants display prolonged longevity and retention of perianth organs. Petals and sepals of the ZmARGOS1 transgenic plants remain turgid (bottom right) while the perianth organs of the flower in the same position on inflorescences abscised in vector control plants (bottom left).

FIG. 14. Effect of ZmARGOS1 Overexpression on the etol-1 Mutant Phenotype in Arabidopsis.

(A) Three-day-old etiolated etol-1 seedlings overexpressing ZmARGOS1 (right) lack the constitutive ethylene response phenotype of the etol-1 mutant (left).

(B) Morphology of light-grown etol-1 mutant plants (right), etol-1 plants overexpressing ZmARGOS1 (left) and vector controls (middle).

FIG. 15. Increased Ethylene Production and Reduced Expression of Ethylene-Inducible Genes in Arabidopsis Overexpressing ZmARGOS1.

(A) Ethylene production in rosette leaves of ZmARGOS1 transgenic events (E1, E2 and E3), vector controls (Vec) and wild-type Col-0 (WT) grown under the light 20 days after planting. Ethylene was collected for a period of 22 hr and subsequently measured using a gas chromatograph. Ethylene production was calculated based on tissue fresh weight. Error bars, standard deviation (n=4).

(B) Down-regulation of ethylene responsive gene expression in transgenic plants overexpressing ZmARGOS1. Total RNA was extracted from rosette leaves of 3-week-old plants. Northern blotting analysis of three ZmARGOS1 events (E1, E2 and E3) and vector controls (Vec) were performed using 10 pg of RNA per lane and probed with ethylene-inducible genes EBF2 and AtERF5. The gel stained with ethidium bromide is shown at the bottom as a control for loading.

FIG. 16. Overexpression of maize ARGOS1 in the ctrl-1 Mutant Background.

(A) Three-day-old etiolated seedlings of ctr1-1 mutant plants overexpressing ZmARGOS1 or vector control displaying the triple response in the absence of exogenous ethylene.

(B) Thirty-day-old ctr1-1 mutant plants overexpressing ZmARGOS1 or vector control displaying the constitutive ethylene response phenotype.

FIG. 17. Overexpression of maize and Arabidopsis TPT domain-containing transmembrane ARGOS proteins confers reduced sensitivity to ethylene.

(A) Reduced ethylene sensitivity phenotype in 3-day-old etiolated seedlings overexpressing maize ZmARGOS1, ZmARGOS9, ZmARGOS8 and ZmARGOS7 and the Arabidopsis homologous gene AtARGOS3 and AtARGOS4. Seedlings were grown in the presence of 10 μM ACC. Representative transgenic T1 seedlings are shown.

(B) Overexpression of Arabidopsis AtARGOS2 reduced sensitivity to ethylene. T3 seedlings of four randomly selected transgenic events (E1-E4) and wild-type Col-0 (WT) were grown in the dark for 3 days in the presence of 0, 1.0 and 2.5 μM ACC. The mean of relative length of hypocotyls and roots is shown for 20 seedlings. The hypotocyl and root length at 0 μM ACC was set as 100%. Asterisks indicate differences between WT and transgenics with statistical significance at P<0.01 (t-test). Error bars, standard deviation (n=20).

FIG. 18. Functional Analysis of Truncated and Mutated ZmARGOS1 in Transgenic Arabidopsis.

(A) Schematic representation of ZmARGOS1 variants. Truncation of the N- and C-terminal sequences of ZmARGOS1 produced TR-n1 (aa 31-144), n2 (aa 62-144) and n3 (aa 92-144) and TR-c1 (aa 1-134), c2 (aa 1-124) and c3 (aa 1-114), respectively. TR-nc (aa 62-134) has the N- and C-terminal sequence truncated. TM1m contains amino acid substitution of P83D and A84D in the first transmembrane domain (TM1). TM2m carries mutation of L120D, L121D and L122D in the second transmembrane domain (TM2). L104D represents single amino acid substitution of L104D in proline-rich motif (PRM).

(B) Measurements of hypocotyl and root length of 3-day-old etiolated seedlings for wild-type control and transgenic Arabidopsis overexpressing ZmARGOS1 and truncated and mutated ZmARGOS1 in the presence of 10 μM ACC. The mean±SD is shown for 12-20 T1 seedlings per construct.

FIG. 19. Single-Amino Acid Substitution Analysis of the Proline-Rich Motif in ZmARGOS1.

Each of the eight amino acids in the proline-rich motif (aa102PPLPPPPS109) of maize ZmARGOS1 gene was substituted with aspartate. The mutant ZmARGOS1 variants and the wild-type ZmARGOS1 were overexpressed in Arabidopsis under the control of the CaMV 35S promoter. Twenty-five T1 seeds were randomly selected for each construct based on the expression of the yellow fluorescent protein marker gene. Ethylene responses were assayed using etiolated seedlings in the presence of 10 μM ACC. Wild-type Col-0 plants (WT) served as controls. Representative seedlings are shown.

FIG. 20. Localization of ZmARGOS1 protein in the ER and Golgi membrane.

(A) Western blotting analysis of cellular fractions of Arabidopsis overexpressing FLAG-HA epitope-tagged ZmARGOS1 (ZmARGOS1) and untagged ZmARGOS1 control (CK). Total (T) homogenates were ultracentrifuged to separate the soluble (S) and micosomal membranes (M) fraction. Western blotting analysis was performed with anti-FLAG antibodies.

(B) Epi-fluorescence microscopy of representative hypocotyl cells of stable transgenic Arabidopsis expressing AcGFP-tagged ZmARGOS1 showing green fluorescence associated with the ER and Golgi membrane.

(C) Co-localization of AcGFP tagged ZmARGOS1 with the ER marker in transiently transformed onion epidermal cells.

(D) Co-localization of AcGFP tagged ZmARGOS1 with the Golgi marker in transiently transformed onion epidermal cells.

FIG. 21. Alignment of ARGOS polypeptide sequences from various species identifying conserved transmembrane segments. Information is labeled as follows:

ID=SEQ ID, although grass sp. are identified per Table 1 as argos #

St=sequence start number in the aligned sequence panel,

Ed=sequence ending number in the aligned sequence panel,

TMH1/2=transmembrane segments,

Ident/TMH1,2=ratio of identity. Alignment produced by Clustalw with ZmARGOS8 (SEQ ID NO: 44) as the aligning profile. The identity calculation is as compared to ZmARGOS8.

FIG. 22. Effect of ZmARGOS8 transgene on plant growth under 2 mM nitrate conditions.

Three UBI:ZmARGOS8 transgenic events and null were grown in 10 liter pots with 2 mM nitrate treatment in the field. Eight plants per event were sampled and the shoot and root biomass in fresh weight (g) was collected. (A) Average shoot (top) and root biomass (bottom) at V7 stage; (B) Average shoot (top) and root biomass (bottom) at R3 stage. Asterisks indicate significance at p<0.05.

FIG. 23. Overexpression of ZmARGOS8 improves maize yields under drought stresses. The graph describes the yield increase in bushels per acre relative to non-transgenic controls for 10 independent events

### DETAILED DESCRIPTION

There is a continuing need for modulation of ethylene sensitivity and ethylene response pathways in plants for manipulating plant development or stress responses.

This disclosure relates to the identification, characterization and manipulation of genes which are used to modulate improve yield and/or stress tolerance in plants. Improvement in yield and/or stress tolerance may be achieved by regulating ethylene sensitivity.

The disclosure includes methods to alter the genetic composition of crop plants, for example maize, so that such crops can be higher yielding and/or more tolerant to stress conditions. The utility of this class of disclosure is then both yield enhancement and stress tolerance through modulation of ethylene sensitivity and/or regulation of ethylene responses.

Regulation of ethylene responses include but are not limited to those involving: crowding tolerance, seed set and development, growth in compacted soils, flooding tolerance, maturation and senescence, drought tolerance and disease resistance. This disclosure provides methods and compositions to effect various alterations in ethylene sensitivity or an ethylene response in a plant that would result in improved agronomic performance in normal or stress conditions. The plants disclosed have altered ethylene sensitivity as compared to a control plant. In some plants, the altered ethylene sensitivity is directed to a vegetative tissue, a reproductive tissue, or a vegetative tissue and a reproductive tissue. Plants of the disclosure can have at least one of the following phenotypes including but not limited to: differences in crowding tolerance, seed set and development, growth in compacted soils, flooding tolerance, drought tolerance, maturation and senescence and disease resistance compared to non transformed plants.

Unless defined otherwise, all technical and scientific terms used herein have the same meaning as commonly understood by one of ordinary skill in the art to which this disclosure belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the disclosure.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

The practice of the present disclosure will employ, unless otherwise indicated, conventional techniques of botany, microbiology, tissue culture, molecular biology, chemistry, biochemistry and recombinant DNA technology, which are within the skill of the art. Such techniques are explained fully in the literature. See, e.g., Langenheim and Thimann, BOTANY: PLANT BIOLOGY AND ITS RELATION TO HUMAN AFFAIRS, John Wiley (1982); CELL CULTURE AND SOMATIC CELL GENETICS OF PLANTS, vol. 1, Vasil, ed. (1984); Stanier, et al., THE MICROBIAL WORLD, 5^{th} ed., Prentice-Hall (1986); Dhringra and Sinclair, BASIC PLANT PATHOLOGY METHODS, CRC Press (1985); Maniatis, et al., MOLECULAR CLONING: A LABORATORY MANUAL (1982); DNA CLONING, vols. I and II, Glover, ed. (1985); OLIGONUCLEOTIDE SYNTHESIS, Gait, ed. (1984); NUCLEIC ACID HYBRIDIZATION, Hames and Higgins, eds. (1984); and the series METHODS IN ENZYMOLOGY, Colowick and Kaplan, eds, Academic Press, Inc., San Diego, Calif.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present disclosure, the following terms will be employed, and are intended to be defined as indicated below.

By “microbe” is meant any microorganism (including both eukaryotic and prokaryotic microorganisms), such as fungi, yeast, bacteria, actinomycetes, algae and protozoa, as well as other unicellular structures.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS) and strand displacement amplification (SDA). See, e.g., DIAGNOSTIC MOLECULAR MICROBIOLOGY: PRINCIPLES AND APPLICATIONS, Persing, et al., eds., American Society for Microbiology, Washington, DC (1993). The product of amplification is termed an amplicon.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32) can be modified to yield a functionally identical molecule. Accordingly, each silent variation of a nucleic acid, which encodes a polypeptide of the present disclosure, is implicit in each described polypeptide sequence and incorporated herein by reference.

As to amino acid sequences, one of skill will recognize that individual substitutions, deletions or additions to a nucleic acid, peptide, polypeptide or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80% or 90%, preferably 60-90% of the native protein for it's native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton, PROTEINS, W.H. Freeman and Co. (1984).

As used herein, “consisting essentially of” means the inclusion of additional sequences to an object polynucleotide where the additional sequences do not selectively hybridize, under stringent hybridization conditions, to the same cDNA as the polynucleotide and where the hybridization conditions include a wash step in 0.1×SSC and 0.1% sodium dodecyl sulfate at 65° C.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present disclosure may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which contains a vector and supports the replication and/or expression of the expression vector. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The terms “isolated” refers to material, such as a nucleic acid or a protein, which is substantially or essentially free from components which normally accompany or interact with it as found in its naturally occurring environment. The isolated material optionally comprises material not found with the material in its natural environment. Nucleic acids, which are “isolated”, as defined herein, are also referred to as “heterologous” nucleic acids. Unless otherwise stated, the term “ARGOS nucleic acid” means a nucleic acid comprising a polynucleotide (“ARGOS polynucleotide”) encoding a ARGOS polypeptide.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, GUIDE TO MOLECULAR CLONING TECHNIQUES, from the series METHODS IN ENZYMOLOGY, vol. 152, Academic Press, Inc., San Diego, Calif. (1987); Sambrook, et al., MOLECULAR CLONING: A LABORATORY MANUAL, 2^{nd} ed., vols. 1-3 (1989); and CURRENT PROTOCOLS IN MOLECULAR BIOLOGY, Ausubel, et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds suspension cultures, embryos, meristematic regions, callus tissue, leaves, roots, shoots, gametophytes, sporophytes, pollen and microspores. The class of plants, which can be used in the methods of the disclosure, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is Zea mays.

As used herein, “yield” includes reference to bushels per acre of a grain crop at harvest, as adjusted for grain moisture (15% typically). Grain moisture is measured in the grain at harvest. The adjusted test weight of grain is determined to be the weight in pounds per bushel, adjusted for grain moisture level at harvest.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Examples are promoters that preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may effect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions.

The term “ARGOS polypeptide” refers to one or more amino acid sequences. The term is also inclusive of fragments, variants, homologs, alleles or precursors (e.g., preproproteins or proproteins) thereof. A “ARGOS protein” comprises a ARGOS polypeptide. Unless otherwise stated, the term “ARGOS nucleic acid” means a nucleic acid comprising a polynucleotide (“ARGOS polynucleotide”) encoding a ARGOS polypeptide.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

The term “residue” or “amino acid residue” or “amino acid” are used interchangeably herein to refer to an amino acid that is incorporated into a protein, polypeptide or peptide (collectively “protein”). The amino acid may be a naturally occurring amino acid and, unless otherwise limited, may encompass known analogs of natural amino acids that can function in a similar manner as naturally occurring amino acids.

It is understood, as those skilled in the art will appreciate, that the invention encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The protein of the current invention may also be a protein which comprises an amino acid sequence comprising deletion, substitution, insertion and/or addition of one or more amino acids in an amino acid sequence selected from the group consisting of SEQ ID NOS listed in Table 1. The substitution may be conservative, which means the replacement of a certain amino acid residue by another residue having similar physical and chemical characteristics. Non-limiting examples of conservative substitution include replacement between aliphatic group-containing amino acid residues such as Ile, Val, Leu or Ala and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research 10(20):6487-6500 (1982), which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

The protein of the present invention may also be a protein which is encoded by a nucleic acid comprising a nucleotide sequence comprising deletion, substitution, insertion and/or addition of one or more nucleotides in a nucleotide sequence selected from the group consisting of SEQ ID NOS listed in Table 1. Nucleotide deletion, substitution, insertion and/or addition may be accomplished by site-directed mutagenesis or other techniques as mentioned above.

The protein of the present invention may also be a protein which is encoded by a nucleic acid comprising a nucleotide sequence hybridizable under stringent conditions with the complementary strand of a nucleotide sequence selected from the group consisting of SEQ ID NOS listed in Table 1.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook, et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6'SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCI, and washing twice in 0.4% SDS, 0.5×SSC at 55° C. for 20 minutes and once in 2×SSC at room temperature for 5 minutes.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence”, (b) “comparison window”, (c) “sequence identity”, (d) “percentage of sequence identity” and (e) “substantial identity”.

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package®, Version 8 (available from Genetics Computer Group (GCG® programs (Accelrys, Inc., San Diego, Calif.)). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65 and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences and TBLASTX for nucleotide query sequences against nucleotide database sequences. See, CURRENT PROTOCOLS IN MOLECULAR BIOLOGY, Chapter 19, Ausubel, et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps.

GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package® are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package® is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

The disclosure discloses ARGOS polynucleotides and polypeptides. The novel nucleotides and proteins of the disclosure have an expression pattern which indicates that they regulate cell number and thus play an important role in plant development. The polynucleotides are expressed in various plant tissues. The polynucleotides and polypeptides thus provide an opportunity to manipulate plant development to alter seed and vegetative tissue development, timing or composition. This may be used to create a sterile plant, a seedless plant or a plant with altered endosperm composition.

### Nucleic Acids

The present disclosure provides, inter alia, isolated nucleic acids of RNA, DNA and analogs and/or chimeras thereof, comprising a ARGOS polynucleotide.

The present disclosure also includes polynucleotides optimized for expression in different organisms. For example, for expression of the polynucleotide in a maize plant, the sequence can be altered to account for specific codon preferences and to alter GC content as according to Murray, et al., supra. Maize codon usage for 28 genes from maize plants is listed in Table 4 of Murray, et al., supra.

The ARGOS nucleic acids of the present disclosure comprise isolated ARGOS polynucleotides which are inclusive of:
(a) a polynucleotide encoding a ARGOS polypeptide and conservatively modified and polymorphic variants thereof;
(b) a polynucleotide having at least 70% sequence identity with polynucleotides of (a) or (b);
(c) complementary sequences of polynucleotides of (a) or (b).

The following table, Table 1, lists the specific identities of the polynucleotides and polypeptides and disclosed herein.

TABLE 1

| | | Polynucleotide/ | |
|---|---|---|---|
| Gene name | Plant species | Polypeptide | SEQ ID NO: |
| ZmARGOS1 | Zea mays | Polynucleotide | SEQ ID NO: 1 |
| | | Polypeptide | SEQ ID NO: 2 |
| | | Genomic sequence | SEQ ID NO: 71 |
| ZmARGOS2 | Zea mays | Polynucleotide | SEQ ID NO: 3 |
| (allelic variant, | | Polypeptide | SEQ ID NO: 4 |
| of ZmARGOS1) | | | |
| ZmARGOS3 | Zea mays | Polynucleotide | SEQ ID NO: 5 |
| | | Polypeptide | SEQ ID NO: 6 |
| ZmARGOS4 | Zea mays | Polypeptide | SEQ ID NO: 7 |
| | | Polynucleotide | SEQ ID NO: 40 |
| ZmARGOS5 | Zea mays | Polypeptide | SEQ ID NO: 8 |
| | | Polynucleotide | SEQ ID NO: 41 |
| ZmARGOS6 | Zea mays | Polypeptide | SEQ ID NO: 9 |
| | | Polynucleotide | SEQ ID NO: 42 |
| ZmARGOS7 | Zea mays | Polypeptide | SEQ ID NO: 10 |
| | | Polynucleotide | SEQ ID NO: 43 |
| ZmARGOS8 | Zea mays | Polypeptide | SEQ ID NO: 11 |
| | | Polynucleotide | SEQ ID NO: 44 |
| ZmARGOS9 | Zea mays | Polypeptide | SEQ ID NO: 12 |
| | | Polynucleotide | SEQ ID NO: 45 |
| OsARGOS1 | Oryza sativa | Polypeptide | SEQ ID NO: 13 |
| | | Polynucleotide | SEQ ID NO: 46 |
| OsARGOS2 | Oryza sativa | Polypeptide | SEQ ID NO: 14 |
| | | Polynucleotide | SEQ ID NO: 47 |
| OsARGOS3 | Oryza sativa | Polypeptide | SEQ ID NO: 15 |
| | | Polynucleotide | SEQ ID NO: 48 |
| OsARGOS4 | Oryza sativa | Polypeptide | SEQ ID NO: 16 |
| | | Polynucleotide | SEQ ID NO: 49 |
| OsARGOS5 | Oryza sativa | Polypeptide | SEQ ID NO: 17 |
| | | Polynucleotide | SEQ ID NO: 50 |
| OsARGOS6 | Oryza sativa | Polypeptide | SEQ ID NO: 18 |
| | | Polynucleotide | SEQ ID NO: 51 |
| OsARGOS7 | Oryza sativa | Polypeptide | SEQ ID NO: 19 |
| | | Polynucleotide | SEQ ID NO: 52 |
| OsARGOS8 | Oryza sativa | Polypeptide | SEQ ID NO: 20 |
| | | Polynucleotide | SEQ ID NO: 53 |
| GmARGOS1 | Glycine max | Polypeptide | SEQ ID NO: 21 |
| | | Polynucleotide | SEQ ID NO: 54 |
| GmARGOS2 | Glycine max | Polypeptide | SEQ ID NO: 22 |
| | | Polynucleotide | SEQ ID NO: 55 |
| GmARGOS3 | Glycine max | Polypeptide | SEQ ID NO: 23 |
| | | Polynucleotide | SEQ ID NO: 56 |
| GmARGOS4 | Glycine max | Polypeptide | SEQ ID NO: 24 |
| | | Polynucleotide | SEQ ID NO: 57 |
| GmARGOS5 | Glycine max | Polypeptide | SEQ ID NO: 25 |
| | | Polynucleotide | SEQ ID NO: 58 |
| SbARGOS1 | Sorghum bicolor | Polypeptide | SEQ ID NO: 29 |
| | | Polynucleotide | SEQ ID NO: 62 |
| SbARGOS2 | Sorghum bicolor | Polypeptide | SEQ ID NO: 30 |
| | | Polynucleotide | SEQ ID NO: 63 |
| SbARGOS3 | Sorghum bicolor | Polypeptide | SEQ ID NO: 31 |
| | | Polynucleotide | SEQ ID NO: 64 |
| SbARGOS4 | Sorghum bicolor | Polypeptide | SEQ ID NO: 32 |
| | | Polynucleotide | SEQ ID NO: 65 |
| SbARGOS5 | Sorghum bicolor | Polypeptide | SEQ ID NO: 33 |
| | | Polynucleotide | SEQ ID NO: 66 |
| SbARGOS6 | Sorghum bicolor | Polypeptide | SEQ ID NO: 34 |
| | | Polynucleotide | SEQ ID NO: 67 |
| SbARGOS7 | Sorghum bicolor | Polypeptide | SEQ ID NO: 35 |
| | | Polynucleotide | SEQ ID NO: 68 |
| SbARGOS8 | Sorghum bicolor | Polypeptide | SEQ ID NO: 36 |
| | | Polynucleotide | SEQ ID NO: 69 |
| SbARGOS9 | Sorghum bicolor | Polypeptide | SEQ ID NO: 37 |
| | | Polynucleotide | SEQ ID NO: 70 |
| AtARGOS1 | Arabidopsis thaliana | Polypeptide | SEQ ID NO: 26 |
| | | Polynucleotide | SEQ ID NO: 59 |
| AtARGOS2 | Arabidopsis thaliana | Polypeptide | SEQ ID NO: 27 |
| | | Polynucleotide | SEQ ID NO: 60 |
| AtARGOS3 | Arabidopsis thaliana | Polypeptide | SEQ ID NO: 28 |
| | | Polynucleotide | SEQ ID NO: 61 |
| Primer | Artificial seguence | Polynucleotide | SEQ ID NO: 38 |
| Primer | Artificial seguence | Polynucleotide | SEQ ID NO: 39 |
| BahiaGrass ARGOS1 | Bahia Grass | Polynucleotide | SEQ ID NO: 72 |
| assm_NODE_91017 | | Polypeptide | SEQ ID NO: 73 |
| BahiaGrass ARGOS9 | Bahia Grass | Polynucleotide | SEQ ID NO: 74 |
| assm_NODE_247924 | | Polypeptide | SEQ ID NO: 75 |
| Bahia Grass ARGOS3 | Bahia Grass | Polynucleotide | SEQ ID NO: 76 |
| 182675_186771_con | | Polypeptide | SEQ ID NO: 77 |
| Bahia Grass ARGOS6 | Bahia Grass | Polynucleotide | SEQ ID NO: 78 |
| assm_NODE_583424 | | Polypeptide | SEQ ID NO: 79 |
| Resurrection Grass | Resurrection Grass | Polynucleotide | SEQ ID NO: 80 |
| ARGOS8 | | Polypeptide | SEQ ID NO: 81 |
| Con2_incomμlete | | | |
| Resurrection Grass | Resurrection Grass | Polynucleotide | SEQ ID NO: 82 |
| ARGOS7 | | Polypeptide | SEQ ID NO: 83 |
| assm_NODE_128576 | | | |
| Sudan Grass Assm Node | Sudan Grass | Polypeptide | SEQ ID NO: 84 |
| 32838 partial | | | |
| Consensus from proline | Artificial Seguence - | Polypeptide | SEQ ID NO: 85 |
| rich region | Consensus | | |
| Consensus from proline | Artificial Seguence - | Polypeptide | SEQ ID NO: 86 |
| rich region with variable | Consensus marked | | |
| regions indicated | variable regions | | |
| Truncated ZmARGOS8 | Zea mays | Polypeptide | SEQ ID NO: 87 |
| Proline rich motif PRM | Zea mays | Polypeptide | SEQ ID NO: 88 |
| ZmARGOS1a | | | |
| TPT domain | Zea mays | Polypeptide | SEQ ID NO: 89 |
| ZmARGOS1a | | | |
| TM1 | Zea mays | Polypeptide | SEQ ID NO: 90 |
| TM2 | Zea mays | Polypeptide | SEQ ID NO: 91 |
| Primer | Artificial seguence | Polypeptide | SEQ ID NO: 92 |
| Linker | Artificial seguence | Polypeptide | SEQ ID NO: 93 |
| 5-prime bar primer | Artificial seguence | Polynucleotide | SEQ ID NO: 94 |
| 3-prime bar primer | Artificial seguence | Polynucleotide | SEQ ID NO: 95 |
| PRM seguence with | Zea mays | Polypeptde | SEQ ID NO: 96 |
| identified variable regions | | | |
| SB04G023130.1 | Sorghum bicolor | Polypeptide | SEQ ID NO: 97 |
| conserved region | | | |
| SB05G0d6900.1 | Sorghum bicolor | Polypeptide | SEQ ID NO: 98 |
| conserved region | | | |
| SB06G017750.1 | Sorghum bicolor | Polypeptide | SEQ ID NO: 99 |
| conserved region | | | |
| SB7G001405.1 | Sorghum bicolor | Polypeptide | SEQ ID NO: 100 |
| conserved region | | | |
| SB09G020520.1 | Sorghum bicolor | Polypeptide | SEQ ID NO: 101 |
| conserved region | | | |
| Variant PRM | Artificial sequence | Polypeptide | SEQ ID NO: 102 |
| AtARGOS4 | Arabidopsis thaliana | Polynucleotide | SEQ ID NO: 103 |
| AtARGOS4 | Arabidopsis thaliana | Polypeptide | SEQ ID NO: 104 |

### Construction of Nucleic Acids

The isolated nucleic acids of the present disclosure can be made using (a) standard recombinant methods, (b) synthetic techniques or combinations thereof. In some embodiments, the polynucleotides of the present disclosure will be cloned, amplified or otherwise constructed from a fungus or bacteria.

### Synthetic Methods for Constructing Nucleic Acids

The isolated nucleic acids of the present disclosure can also be prepared by direct chemical synthesis by methods such as the phosphotriester method of Narang, et al., (1979) Meth. Enzymol. 68:90-9; the phosphodiester method of Brown, et al., (1979) Meth. Enzymol. 68:109-51; the diethylphosphoramidite method of Beaucage, et al., (1981) Tetra. Letts. 22(20):1859-62; the solid phase phosphoramidite triester method described by Beaucage, et al., supra, e.g., using an automated synthesizer, e.g., as described in Needham-VanDevanter, et al., (1984) Nucleic Acids Res. 12:6159-68 and the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G>7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present disclosure provides 5′ and/or 3′ UTR regions for modulation of translation of heterologous coding sequences.

Further, the polypeptide-encoding segments of the polynucleotides of the present disclosure can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present disclosure can be analyzed statistically using commercially available software packages such as “Codon Preference” available from the University of Wisconsin Genetics Computer Group. See, Devereaux, et al., (1984) Nucleic Acids Res. 12:387-395; or MacVector 4.1 (Eastman Kodak Co., New Haven, Conn.). Thus, the present disclosure provides a codon usage frequency characteristic of the coding region of at least one of the polynucleotides of the present disclosure. The number of polynucleotides (3 nucleotides per amino acid) that can be used to determine a codon usage frequency can be any integer from 3 to the number of polynucleotides of the present disclosure as provided herein. Optionally, the polynucleotides will be full-length sequences. An exemplary number of sequences for statistical analysis can be at least 1, 5, 10, 20, 50 or 100.

### Sequence Shuffling

The present disclosure provides methods for sequence shuffling using polynucleotides of the present disclosure, and compositions resulting therefrom. Sequence shuffling is described in PCT Publication Number 1996/19256. See also, Zhang, et al., (1997) Proc. Natl. Acad. Sci. USA 94:4504-9 and Zhao, et al., (1998) Nature Biotech 16:258-61. Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation or other expression property of a gene or transgene, a replicative element, a protein-binding element or the like, such as any feature which confers a selectable or detectable property. In some embodiments, the selected characteristic will be an altered Kₘ and/or K_{cat} over the wild-type protein as provided herein. In other embodiments, a protein or polynucleotide generated from sequence shuffling will have a ligand binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

### Recombinant Expression Cassettes

The present disclosure further provides recombinant expression cassettes comprising a nucleic acid of the present disclosure. A nucleic acid sequence coding for the desired polynucleotide of the present disclosure, for example a cDNA or a genomic sequence encoding a polypeptide long enough to code for an active protein of the present disclosure, can be used to construct a recombinant expression cassette which can be introduced into the desired host cell. A recombinant expression cassette will typically comprise a polynucleotide of the present disclosure operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site and/or a polyadenylation signal.

A plant promoter fragment can be employed which will direct expression of a polynucleotide of the present disclosure in all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30) and maize H3 histone (Lepetit, et al., (1992) Mol. Gen. Genet. 231:276-85 and Atanassvoa, et al., (1992) Plant Journal 2(3):291-300); ALS promoter, as described in PCT Application Number WO 1996/30530; GOS2 (U.S. Pat. No. 6,504,083) and other transcription initiation regions from various plant genes known to those of skill. For the present disclosure ubiquitin is the preferred promoter for expression in monocot plants.

Alternatively, the plant promoter can direct expression of a polynucleotide of the present disclosure in a specific tissue or may be otherwise under more precise environmental or developmental control. Such promoters are referred to here as “inducible” promoters (Rab17, RAD29). Environmental conditions that may effect transcription by inducible promoters include pathogen attack, anaerobic conditions, or the presence of light. Examples of inducible promoters are the Adh1 promoter, which is inducible by hypoxia or cold stress, the Hsp70 promoter, which is inducible by heat stress, and the PPDK promoter, which is inducible by light.

Examples of promoters under developmental control include promoters that initiate transcription only, or preferentially, in certain tissues, such as leaves, roots, fruit, seeds or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of plant genes, or from T-DNA. The 3′ end sequence to be added can be derived from, for example, the nopaline synthase or octopine synthase genes or alternatively from another plant gene or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50 and An, et al., (1989) Plant Cell 1:115-22) and the CaMV 19S gene (Mogen, et al., (1990) Plant Cell 2:1261-72).

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2 and 6, the Bronze-1 intron are known in the art. See generally, THE MAIZE HANDBOOK, Chapter 116, Freeling and Walbot, eds., Springer, N.Y. (1994).

Plant signal sequences, including, but not limited to, signal-peptide encoding DNA/RNA sequences which target proteins to the extracellular matrix of the plant cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant Cell, 2:301-13); signal peptides which cause proteins to be secreted, such as that of PRIb (Lind, et al., (1992) Plant Mol. Biol. 18:47-53) or the barley alpha amylase (BAA) (Rahmatullah, et al., (1989) Plant Mol. Biol. 12:119 and hereby incorporated by reference) or signal peptides which target proteins to the plastids such as that of rapeseed enoyl-Acp reductase (Verwaert, et al., (1994) Plant Mol. Biol. 26:189-202) are useful in the disclosure. The barley alpha amylase signal sequence fused to the ARGOS polynucleotide is the preferred construct for expression in maize for the present disclosure.

The vector comprising the sequences from a polynucleotide of the present disclosure will typically comprise a marker gene, which confers a selectable phenotype on plant cells. Usually, the selectable marker gene will encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene) or other such genes known in the art. The bar gene encodes resistance to the herbicide basta and the ALS gene encodes resistance to the herbicide chlorsulfuron.

Typical vectors useful for expression of genes in higher plants are well known in the art and include vectors derived from the tumor-inducing (Ti) plasmid of Agrobacterium tumefaciens described by Rogers, et al., (1987) Meth. Enzymol. 153:253-77. These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl, et al., (1987) Gene 61:1-11 and Berger, et al., (1989) Proc. Natl. Acad. Sci. USA, 86:8402-6. Another useful vector herein is plasmid pBI101.2 that is available from CLONTECH Laboratories, Inc. (Palo Alto, Calif.).

### Expression of Proteins in Host Cells

Using the nucleic acids of the present disclosure, one may express a protein of the present disclosure in a recombinantly engineered cell such as bacteria, yeast, insect, mammalian or preferably plant cells. The cells produce the protein in a non-natural condition (e.g., in quantity, composition, location and/or time), because they have been genetically altered through human intervention to do so.

It is expected that those of skill in the art are knowledgeable in the numerous expression systems available for expression of a nucleic acid encoding a protein of the present disclosure. No attempt to describe in detail the various methods known for the expression of proteins in prokaryotes or eukaryotes will be made.

In brief summary, the expression of isolated nucleic acids encoding a protein of the present disclosure will typically be achieved by operably linking, for example, the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present disclosure. To obtain high level expression of a cloned gene, it is desirable to construct expression vectors which contain, at the minimum, a strong promoter, such as ubiquitin, to direct transcription, a ribosome binding site for translational initiation and a transcription/translation terminator. Constitutive promoters are classified as providing for a range of constitutive expression. Thus, some are weak constitutive promoters, and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level” or about 1/10 transcripts to about 1/100 transcripts to about 1/1,000 transcripts.

One of skill would recognize that modifications could be made to a protein of the present disclosure without diminishing its biological activity. Some modifications may be made to facilitate the cloning, expression or incorporation of the targeting molecule into a fusion protein. Such modifications are well known to those of skill in the art and include, for example, a methionine added at the amino terminus to provide an initiation site or additional amino acids (e.g., poly His) placed on either terminus to create conveniently located restriction sites or termination codons or purification sequences.

### Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present disclosure are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present disclosure.

### Expression in Eukaryotes

A variety of eukaryotic expression systems such as yeast, insect cell lines, plant and mammalian cells, are known to those of skill in the art. As explained briefly below, the present disclosure can be expressed in these eukaryotic systems. In some embodiments, transformed/transfected plant cells, as discussed infra, are employed as expression systems for production of the proteins of the instant disclosure.

Synthesis of heterologous proteins in yeast is well known. Sherman, et al., (1982) METHODS IN YEAST GENETICS, Cold Spring Harbor Laboratory is a well recognized work describing the various methods available to produce the protein in yeast. Two widely utilized yeasts for production of eukaryotic proteins are Saccharomyces cerevisiae and Pichia pastoris. Vectors, strains and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase and an origin of replication, termination sequences and the like as desired.

A protein of the present disclosure, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

Appropriate vectors for expressing proteins of the present disclosure in insect cells are usually derived from the SF9 baculovirus. Suitable insect cell lines include mosquito larvae, silkworm, armyworm, moth and Drosophila cell lines such as a Schneider cell line (see, e.g., Schneider, (1987) J. Embryol. Exp. Morphol. 27:353-65).

As with yeast, when higher animal or plant host cells are employed, polyadenlyation or transcription terminator sequences are typically incorporated into the vector. An example of a terminator sequence is the polyadenlyation sequence from the bovine growth hormone gene. Sequences for accurate splicing of the transcript may also be included. An example of a splicing sequence is the VP1 intron from SV40 (Sprague, et al., (1983) J. Virol. 45:773-81). Additionally, gene sequences to control replication in the host cell may be incorporated into the vector such as those found in bovine papilloma virus type-vectors (Saveria-Campo, “Bovine Papilloma Virus DNA a Eukaryotic Cloning Vector,” in DNA CLONING: A PRACTICAL APPROACH, vol. II, Glover, ed., IRL Press, Arlington, Va., pp. 213-38 (1985)).

In addition, the gene for ARGOS placed in the appropriate plant expression vector can be used to transform plant cells. The polypeptide can then be isolated from plant callus or the transformed cells can be used to regenerate transgenic plants. Such transgenic plants can be harvested and the appropriate tissues (seed or leaves, for example) can be subjected to large scale protein extraction and purification techniques.

### Plant Transformation Methods

Numerous methods for introducing foreign genes into plants are known and can be used to insert a ARGOS polynucleotide into a plant host, including biological and physical plant transformation protocols. See, e.g., Miki, et al., “Procedure for Introducing Foreign DNA into Plants,” in METHODS IN PLANT MOLECULAR BIOLOGY AND BIOTECHNOLOGY, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch, et al., (1985) Science 227:1229-31), electroporation, micro-injection and biolistic bombardment.

Expression cassettes and vectors and in vitro culture methods for plant cell or tissue transformation and regeneration of plants are known and available. See, e.g., Gruber, et al., “Vectors for Plant Transformation,” in METHODS IN PLANT MOLECULAR BIOLOGY AND BIOTECHNOLOGY, supra, pp. 89-119.

The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski, et al., (1984) EMBO J. 3:2717-2722) and ballistic particle acceleration (see, for example, U.S. Pat. No. 4,945,050; WO 1991/10725 and McCabe, et al., (1988) Biotechnology 6:923-926). Also see, Tomes, et al., Direct DNA Transfer into Intact Plant Cells Via Microprojectile Bombardment. pp. 197-213 in Plant Cell, Tissue and Organ Culture, Fundamental Methods eds. Gamborg and Phillips, Springer-Verlag Berlin Heidelberg N.Y. 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 1991/10725 (maize); Klein, et al., (1988) Plant Physiol. 91:440-444 (maize); Fromm, et al., (1990) Biotechnology 8:833-839 and Gordon-Kamm, et al., (1990) Plant Cell 2:603-618 (maize); Hooydaas-Van Slogteren and Hooykaas, (1984) Nature (London) 311:763-764; Bytebier, et al., (1987) Proc. Natl. Acad. Sci. USA 84:5345-5349 (Liliaceae); De Wet, et al., (1985) In The Experimental Manipulation of Ovule Tissues, ed. Chapman, et al., pp. 197-209; Longman, N.Y. (pollen); Kaeppler, et al., (1990) Plant Cell Reports 9:415-418; and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185), all of which are herein incorporated by reference.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993 and Simpson, et al., (1986) Plant Mol. Biol. 6:403-15 (also referenced in the '306 patent), all incorporated by reference in their entirety. Once constructed, these plasmids can be placed into A. rhizogenes or A. tumefaciens and these vectors used to transform cells of plant species, which are ordinarily susceptible to Fusarium or Alternaria infection. Several other transgenic plants are also contemplated by the present disclosure including but not limited to soybean, corn, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledonous plants, some gymnosperms and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae, and Chenopodiaceae. Monocot plants can now be transformed with some success. EP Patent Application Number 604 662 A1 discloses a method for transforming monocots using Agrobacterium. EP Patent Application Number 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, (1985) Theor. Appl. Genet. 69:235-40; U.S. Pat. No. 4,658,082; Simpson, et al., supra; and U.S. patent application Ser. Nos. 913,913 and 913,914, both filed Oct. 1, 1986, as referenced in U.S. Pat. No. 5,262,306, issued November 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206 and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731 and Christou, et al., (1987) Proc. Natl. Acad. Sci. USA 84:3962. Direct uptake of DNA into protoplasts using CaCl₂ precipitation, polyvinyl alcohol, or poly-L-ornithine has also been reported. See, e.g., Hain, et al., (1985) Mol. Gen. Genet. 199:161 and Draper, et al., (1982) Plant Cell Physiol. 23:451.

Electroporation of protoplasts and whole cells and tissues has also been described. See, e.g., Donn, et al., (1990) in Abstracts of the VIIth Int'l. Congress on Plant Cell and Tissue Culture IAPTC, A2-38, p. 53; D'Halluin, et al., (1992) Plant Cell 4:1495-505 and Spencer, et al., (1994) Plant Mol. Biol. 24:51-61.

Increasing the Activity and/or Level of a ARGOS Polypeptide

Methods are provided to increase the activity and/or level of the ARGOS polypeptide of the disclosure. An increase in the level and/or activity of the ARGOS polypeptide of the disclosure can be achieved by providing to the plant an ARGOS polypeptide. The ARGOS polypeptide can be provided by introducing the amino acid sequence encoding the ARGOS polypeptide into the plant, introducing into the plant a nucleotide sequence encoding an ARGOS polypeptide or alternatively by modifying a genomic locus encoding the ARGOS polypeptide of the disclosure.

As discussed elsewhere herein, many methods are known the art for providing a polypeptide to a plant including, but not limited to, direct introduction of the polypeptide into the plant, introducing into the plant (transiently or stably) a polynucleotide construct encoding a polypeptide having cell number regulator activity. It is also recognized that the methods of the disclosure may employ a polynucleotide that is not capable of directing, in the transformed plant, the expression of a protein or an RNA. Thus, the level and/or activity of an ARGOS polypeptide may be increased by altering the gene encoding the ARGOS polypeptide or its promoter. See, e.g., Kmiec, U.S. Pat. No. 5,565,350; Zarling, et al., PCT/US93/03868. Therefore mutagenized plants that carry mutations in ARGOS genes, where the mutations increase expression of the ARGOS gene or increase the plant growth and/or organ development activity of the encoded ARGOS polypeptide are provided.

### Crowding Tolerance

The agronomic performance of crop plants is often a function of how well they tolerate planting density. Overcrowded plants grow poorly, hence the age-old practice of thinning and controlled planting density. The stress of overcrowding can be due to simple limitations of nutrients, water, and sunlight. Crowding stress may also be due to enhanced contact between plants. Plants often respond to physical contact by slowing growth and thickening their tissues.

Ethylene has been implicated in plant crowding tolerance. For example, ethylene insensitive tobacco plants did not slow growth when contacting neighboring plants (Knoester, et al., (1998) PNAS USA 95:1933-1937). There is also evidence that ethylene, and the plant's response to it, is involved in water deficit stress, and that ethylene may be causing changes in the plant that limit its growth and aggravate the symptoms of drought stress beyond the loss of water itself.

The present disclosure provides for decreasing ethylene sensitivity in a plant, in particular cereals such as maize, by providing for and/or modulating the expression/activity of one or more ARGOS polynucleotides or their protein products to promote tolerance of close spacing with reduced stress and yield loss. Argos expressing plants disclosed herein can be planted at a higher planting density in the field.

### Seed Set and Development in Maize

Ethylene plays a number of roles in seed development. For example, in maize ethylene is linked to programmed cell death of developing endosperm cells (Young, et al., (1997) Plant Physiol 115:737-751). In addition, ethylene is linked to kernel abortion, such as occurs at the tips of ears, especially in plants grown under stressful conditions (Cheng and Lur, (1997) Physiol. Plant 98:245-252). Reduced kernel seed set is of course a contributor to reduced yields. Consequently, the present disclosure provides plants, in particular maize plants that have reduced ethylene sensitivity by providing for the overexpression of polynucleotides of the disclosure in transgenic plants.

### Growth in Compacted Soils

Plant growth is affected by the density and compaction of soils. Denser, more compacted soils typically result in poorer plant growth. The trend in agriculture towards more minimal till planting and cultivation practices, with the goal of soil and energy conservation, is increasing the need for crop plants that can perform well under these conditions.

Ethylene is well-known to affect plant growth and development and one effect of ethylene is to promote tissue thickening and growth retardation when encountering mechanical stress, such as compacted soils. This can affect both the roots and shoots. This effect is presumably adaptive in some circumstances in that it results in stronger, more compact tissues that can force their way through or around, obstacles such as compacted soils. However, in such conditions, the production of ethylene and the activation of the ethylene pathway may exceed what is needed for adaptive accommodation to the mechanical stress of the compacted soils. And of course, any resulting unnecessary growth inhibition would be an undesired agronomic result.

The present disclosure provides for decreasing ethylene sensitivity in a plant, in particular cereals such as maize, by providing for and/or modulating the expression/activity of one or more polynucleotides or their protein products. Such modulated plants grow and germinate better in compacted soils, resulting in higher stand counts, the herald of higher yields.

### Flooding Tolerance

Flooding and water-logged soils causes substantial losses in crop yield each year around the world. Flooding can be both widespread or local, transitory or prolonged. Ethylene has been implicated in flooding mediated damage. In fact, in flooded conditions ethylene production can rise. There are two main reasons for this rise: 1) under such flooded conditions, which creates hypoxia, plants produce more ethylene and 2) under flooded conditions the diffusion of ethylene away from the plant is slowed, because ethylene is minimally soluble in water, resulting in a rise of intra-plant ethylene levels.

Ethylene in flooded maize roots can also inhibit gravitropism, which is normally adaptive during germination in that it orients the roots down and the shoots up. Gravitropism is a factor in determining root architecture, which in turn plays an important role in soil resource acquisition. Manipulation of ethylene levels could be used to impact root angle for drought tolerance, flood tolerance, greater standability and/or improved nutrient uptake. For example, a root growing at a more erect angle (steeper) would likely grow more deeply in soil and thus obtain water at greater depths, improving drought tolerance. In the absence of drought stress a converse argument could be made for more efficient root uptake of nutrients and water in the upper layers of the soil profile, by roots which are more parallel to the soil surface. In general, roots that have a angle nearer that of vertical (steep) are also more susceptible to root lodging than roots with a shallow angle (parallel to the surface) that can be more root lodging resistant.

In addition to inhibition of gravitropism, it is likely that ethylene evolution in flooded conditions inhibits growth, especially of roots. Such inhibition will likely contribute to poor plant growth overall, and consequently is a disadvantageous agronomic trait.

The present disclosure provides for decreasing ethylene sensitivity in a plant, in particular cereals such as maize, by providing for and/or modulating the expression/activity of one or more polynucleotides or their protein products. Such plants should grow and germinate better in flooded conditions or water-logged soils, resulting in higher stand counts.

### Plant Maturation and Senescence

Ethylene is known to be involved in controlling senescence, fruit ripening, and abscission. The role of ethylene in fruit ripening is well-established and industrially applied.

The prediction based on precedent would be that ethylene underproduction/insensitivity would result in slower seed ripening, and the converse would result in more rapid seed ripening. Abscission is primarily studied for dicot plants and apparently has little application to monocots such as cereals. Ethylene mediated senescence also is mostly studied in dicots, but control of senescence is a agronomically important for both dicot and monocot crop species. Ethylene insensitivity can cause a delay of, but not arrest, senescence. The senescence process mediated by ethylene bears some similarities to the cell death process in disease symptoms and in abscission zones.

Controlling ethylene sensitivity, as through the control of one or more polynucleotides of the disclosure could result in modulation of maturity rates for crop plants such as maize.

The present disclosure provides for decreasing ethylene sensitivity in a plant, in particular cereals such as maize, by providing for and/or modulating the expression/activity of one or more polynucleotides or their protein products which may contribute to a later maturing plant, which is desirable for placing crop varieties in different maturity zones.

### Tolerance to Other Abiotic Stresses

Many stresses on plants cause an induction in the production of ethylene (see, Morgan and Drew, (1997) Physiol. Plant 100:620-630). These stresses can be cold, heat, wounding, pollution, drought, and hypersalinity. Mechanical impedance (soil compaction) and flooding stresses were addressed above. It appears that several of these stresses operate through common mechanisms, such as water deficit. Clearly drought causes water deficit, crowding stress may also cause water deficit. Additionally, in maize chilling can cause an elevation in ethylene production and activity, and this induction is apparently due to chilling causing water deficit in cells (Janowaik and Dorffling, (1995) J. Plant Physiol. 147:257-262).

Some of the ethylene production following stresses may serve an adaptive purpose by regulating ethylene-mediated processes in the plant that result in a plant reorganized in such manner to better acclimate to the stress encountered. However, there is also evidence that ethylene production during stress can result in an aggravation of negative symptoms resulting from the stress, such as yellowing, tissue death and senescence.

To the extent that ethylene production during stress causes or augments negative stress-related symptoms, it would be desirable to create a crop plant that is less sensitive to the ethylene. Towards that end, the present disclosure provides for decreasing ethylene sensitivity in a plant, in particular cereals such as maize, by providing for and/or modulating the expression/activity of one or more polynucleotides or their protein products to create plants that are less sensitive to ethylene mediated effects.

### Kits for Modulating Plant Stress Response

Certain embodiments of the disclosure can optionally be provided to a user as a kit. For example, a kit of the disclosure can contain one or more nucleic acid, polypeptide, antibody, diagnostic nucleic acid or polypeptide, e.g., antibody, probe set, e.g., as a cDNA microarray, one or more vector and/or cell line described herein. Most often, the kit is packaged in a suitable container. The kit typically further comprises one or more additional reagents, e.g., substrates, labels, primers, or the like for labeling expression products, tubes and/or other accessories, reagents for collecting samples, buffers, hybridization chambers, cover slips, etc. The kit optionally further comprises an instruction set or user manual detailing preferred methods of using the kit components for discovery or application of gene sets. When used according to the instructions, the kit can be used, e.g., for evaluating expression or polymorphisms in a plant sample, e.g., for evaluating ethylene sensitivity, stress response potential, crowding resistance potential, sterility, etc. Alternatively, the kit can be used according to instructions for using at least one polynucleotide sequence to control ethylene sensitivity in a plant.

Reducing the Activity and/or Level of a ARGOS Polypeptide

Methods are provided to reduce or eliminate the activity of an ARGOS polypeptide of the disclosure by transforming a plant cell with an expression cassette that expresses a polynucleotide that inhibits the expression of the ARGOS polypeptide. The polynucleotide may inhibit the expression of the ARGOS polypeptide directly, by preventing translation of the ARGOS messenger RNA, or indirectly, by encoding a polypeptide that inhibits the transcription or translation of a ARGOS gene encoding a ARGOS polypeptide. Methods for inhibiting or eliminating the expression of a gene in a plant are well known in the art, and any such method may be used in the present disclosure to inhibit the expression of an ARGOS polypeptide.

In accordance with the present disclosure, the expression of a ARGOS polypeptide is inhibited if the protein level of the ARGOS polypeptide is less than 70% of the protein level of the same ARGOS polypeptide in a plant that has not been genetically modified or mutagenized to inhibit the expression of that ARGOS polypeptide. In particular embodiments of the disclosure, the protein level of the ARGOS polypeptide in a modified plant according to the disclosure is less than 60%, less than 50%, less than 40%, less than 30%, less than 20%, less than 10%, less than 5% or less than 2% of the protein level of the same ARGOS polypeptide in a plant that is not a mutant or that has not been genetically modified to inhibit the expression of that ARGOS polypeptide. The expression level of the ARGOS polypeptide may be measured directly, for example, by assaying for the level of ARGOS polypeptide expressed in the plant cell or plant, or indirectly, for example, by measuring the plant growth and/or organ development activity of the ARGOS polypeptide in the plant cell or plant or by measuring the biomass in the plant. Methods for performing such assays are described elsewhere herein.

In other embodiments of the disclosure, the activity of the ARGOS polypeptides is reduced or eliminated by transforming a plant cell with an expression cassette comprising a polynucleotide encoding a polypeptide that inhibits the activity of a ARGOS polypeptide. The plant growth and/or organ development activity of a ARGOS polypeptide is inhibited according to the present disclosure if the plant growth and/or organ development activity of the ARGOS polypeptide is less than 70% of the plant growth and/or organ development activity of the same ARGOS polypeptide in a plant that has not been modified to inhibit the plant growth and/or organ development activity of that ARGOS polypeptide. In particular embodiments of the disclosure, the plant growth and/or organ development activity of the ARGOS polypeptide in a modified plant according to the disclosure is less than 60%, less than 50%, less than 40%, less than 30%, less than 20%, less than 10% or less than 5% of the plant growth and/or organ development activity of the same ARGOS polypeptide in a plant that that has not been modified to inhibit the expression of that ARGOS polypeptide. The plant growth and/or organ development activity of an ARGOS polypeptide is “eliminated” according to the disclosure when it is not detectable by the assay methods described elsewhere herein. Methods of determining the plant growth and/or organ development activity of an ARGOS polypeptide are described elsewhere herein.

In other embodiments, the activity of an ARGOS polypeptide may be reduced or eliminated by disrupting the gene encoding the ARGOS polypeptide. The disclosure encompasses mutagenized plants that carry mutations in ARGOS genes, where the mutations reduce expression of the ARGOS gene or inhibit the plant growth and/or organ development activity of the encoded ARGOS polypeptide.

Thus, many methods may be used to reduce or eliminate the activity of an ARGOS polypeptide. In addition, more than one method may be used to reduce the activity of a single ARGOS polypeptide. Non-limiting examples of methods of reducing or eliminating the expression of ARGOS polypeptides are given below.

1. Polynucleotide-Based Methods:

In some embodiments of the present disclosure, a plant is transformed with an expression cassette that is capable of expressing a polynucleotide that inhibits the expression of an ARGOS polypeptide of the disclosure. The term “expression” as used herein refers to the biosynthesis of a gene product, including the transcription and/or translation of said gene product. For example, for the purposes of the present disclosure, an expression cassette capable of expressing a polynucleotide that inhibits the expression of at least one ARGOS polypeptide is an expression cassette capable of producing an RNA molecule that inhibits the transcription and/or translation of at least one ARGOS polypeptide of the disclosure. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of an ARGOS polypeptide are given below.

i. Sense Suppression/Cosuppression

In some embodiments of the disclosure, inhibition of the expression of a ARGOS polypeptide may be obtained by sense suppression or cosuppression. For cosuppression, an expression cassette is designed to express an RNA molecule corresponding to all or part of a messenger RNA encoding an ARGOS polypeptide in the “sense” orientation. Over expression of the RNA molecule can result in reduced expression of the native gene. Accordingly, multiple plant lines transformed with the cosuppression expression cassette are screened to identify those that show the greatest inhibition of ARGOS polypeptide expression.

The polynucleotide used for cosuppression may correspond to all or part of the sequence encoding the ARGOS polypeptide, all or part of the 5’ and/or 3′ untranslated region of an ARGOS polypeptide transcript or all or part of both the coding sequence and the untranslated regions of a transcript encoding an ARGOS polypeptide. In some embodiments where the polynucleotide comprises all or part of the coding region for the ARGOS polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657, each of which is herein incorporated by reference. The efficiency of cosuppression may be increased by including a poly-dT region in the expression cassette at a position 3′ to the sense sequence and 5′ of the polyadenylation signal. See, US Patent Application Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See U.S. Pat. Nos. 5,283,184 and 5,034,323, herein incorporated by reference.

ii. Antisense Suppression

In some embodiments of the disclosure, inhibition of the expression of the ARGOS polypeptide may be obtained by antisense suppression. For antisense suppression, the expression cassette is designed to express an RNA molecule complementary to all or part of a messenger RNA encoding the ARGOS polypeptide. Over expression of the antisense RNA molecule can result in reduced expression of the native gene. Accordingly, multiple plant lines transformed with the antisense suppression expression cassette are screened to identify those that show the greatest inhibition of ARGOS polypeptide expression.

The polynucleotide for use in antisense suppression may correspond to all or part of the complement of the sequence encoding the ARGOS polypeptide, all or part of the complement of the 5′ and/or 3′ untranslated region of the ARGOS transcript or all or part of the complement of both the coding sequence and the untranslated regions of a transcript encoding the ARGOS polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 2002/0048814, herein incorporated by reference.

iii. Double-Stranded RNA Interference

In some embodiments of the disclosure, inhibition of the expression of a ARGOS polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of ARGOS polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743, and WO 1999/49029, WO 1999/53050, WO 1999/61631 and WO 2000/49035, each of which is herein incorporated by reference.

iv. Hairpin RNA Interference and Intron-Containing Hairpin RNA Interference

In some embodiments of the disclosure, inhibition of the expression of one or a ARGOS polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38.

Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7 and US Patent Application Publication Number 2003/0175965, each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295 and US Patent Application Publication Number 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 2002/00904, herein incorporated by reference.

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the ARGOS polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,646,805, each of which is herein incorporated by reference.

vi. Ribozymes

In some embodiments, the polynucleotide expressed by the expression cassette of the disclosure is catalytic RNA or has ribozyme activity specific for the messenger RNA of the ARGOS polypeptide. Thus, the polynucleotide causes the degradation of the endogenous messenger RNA, resulting in reduced expression of the ARGOS polypeptide. This method is described, for example, in U.S. Pat. No. 4,987,071, herein incorporated by reference.

vii. Small Interfering RNA or Micro RNA

In some embodiments of the disclosure, inhibition of the expression of a ARGOS polypeptide may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example, Javier, et al., (2003) Nature 425:257-263, herein incorporated by reference. For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of ARGOS expression, the 22-nucleotide sequence is selected from a ARGOS transcript sequence and contains 22 nucleotides of said ARGOS sequence in sense orientation and 21 nucleotides of a corresponding antisense sequence that is complementary to the sense sequence. miRNA molecules are highly efficient at inhibiting the expression of endogenous genes and the RNA interference they induce is inherited by subsequent generations of plants.

2. Polypeptide-Based Inhibition of Gene Expression

In one embodiment, the polynucleotide encodes a zinc finger protein that binds to a gene encoding an ARGOS polypeptide, resulting in reduced expression of the gene. In particular embodiments, the zinc finger protein binds to a regulatory region of an ARGOS gene. In other embodiments, the zinc finger protein binds to a messenger RNA encoding an ARGOS polypeptide and prevents its translation. Methods of selecting sites for targeting by zinc finger proteins have been described, for example, in U.S. Pat. No. 6,453,242 and methods for using zinc finger proteins to inhibit the expression of genes in plants are described, for example, in US Patent Application Publication Number 2003/0037355, each of which is herein incorporated by reference.

3. Polypeptide-Based Inhibition of Protein Activity

In some embodiments of the disclosure, the polynucleotide encodes an antibody that binds to at least one ARGOS polypeptide and reduces the cell number regulator activity of the ARGOS polypeptide. In another embodiment, the binding of the antibody results in increased turnover of the antibody-ARGOS complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

4. Gene Disruption

In some embodiments of the present disclosure, the activity of an ARGOS polypeptide is reduced or eliminated by disrupting the gene encoding the ARGOS polypeptide. The gene encoding the ARGOS polypeptide may be disrupted by any method known in the art. For example, in one embodiment, the gene is disrupted by transposon tagging. In another embodiment, the gene is disrupted by mutagenizing plants using random or targeted mutagenesis and selecting for plants that have reduced cell number regulator activity.

i. Transposon Tagging

In one embodiment of the disclosure, transposon tagging is used to reduce or eliminate the ARGOS activity of one or more ARGOS polypeptide. Transposon tagging comprises inserting a transposon within an endogenous ARGOS gene to reduce or eliminate expression of the ARGOS polypeptide. “ARGOS gene” is intended to mean the gene that encodes an ARGOS polypeptide according to the disclosure.

In this embodiment, the expression of one or more ARGOS polypeptide is reduced or eliminated by inserting a transposon within a regulatory region or coding region of the gene encoding the ARGOS polypeptide. A transposon that is within an exon, intron, 5′ or 3′ untranslated sequence, a promoter or any other regulatory sequence of a ARGOS gene may be used to reduce or eliminate the expression and/or activity of the encoded ARGOS polypeptide.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928). In addition, the TUSC process for selecting Mu insertions in selected genes has been described in Bensen, et al., (1995) Plant Cell 7:75-84; Mena, et al., (1996) Science 274:1537-1540 and U.S. Pat. No. 5,962,764, each of which is herein incorporated by reference.

ii. Mutant Plants with Reduced Activity

Additional methods for decreasing or eliminating the expression of endogenous genes in plants are also known in the art and can be similarly applied to the instant disclosure. These methods include other forms of mutagenesis, such as ethyl methanesulfonate-induced mutagenesis, deletion mutagenesis and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874 and Quesada, et al., (2000) Genetics 154:421-436, each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant disclosure. See, McCallum, et al., (2000) Nat. Biotechnol. 18:455-457, herein incorporated by reference.

Mutations that impact gene expression or that interfere with the function (cell number regulator activity) of the encoded protein are well known in the art. Insertional mutations in gene exons usually result in null-mutants. Mutations in conserved residues are particularly effective in inhibiting the cell number regulator activity of the encoded protein. Conserved residues of plant ARGOS polypeptides suitable for mutagenesis with the goal to eliminate cell number regulator activity have been described. Such mutants can be isolated according to well-known procedures and mutations in different ARGOS loci can be stacked by genetic crossing. See, for example, Gruis, et al., (2002) Plant Cell 14:2863-2882.

In another embodiment of this disclosure, dominant mutants can be used to trigger RNA silencing due to gene inversion and recombination of a duplicated gene locus. See, for example, Kusaba, et al., (2003) Plant Cell 15:1455-1467.

The disclosure encompasses additional methods for reducing or eliminating the activity of one or more ARGOS polypeptide. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides and recombinogenic oligonucleobases. Such vectors and methods of use are known in the art. See, for example, U.S. Pat. Nos. 5,565,350; 5,731,181; 5,756,325; 5,760,012; 5,795,972 and 5,871,984, each of which are herein incorporated by reference. See also, WO 1998/49350, WO 1999/07865, WO 1999/25821 and Beetham, et al., (1999) Proc. Natl. Acad. Sci. USA 96:8774-8778, each of which is herein incorporated by reference.

iii. Modulating Plant Growth and/or Organ Development Activity

In specific methods, the level and/or activity of a cell number regulator in a plant is increased by increasing the level or activity of the ARGOS polypeptide in the plant. Methods for increasing the level and/or activity of ARGOS polypeptides in a plant are discussed elsewhere herein. Briefly, such methods comprise providing a ARGOS polypeptide of the disclosure to a plant and thereby increasing the level and/or activity of the ARGOS polypeptide. In other embodiments, an ARGOS nucleotide sequence encoding an ARGOS polypeptide can be provided by introducing into the plant a polynucleotide comprising an ARGOS nucleotide sequence of the disclosure, expressing the ARGOS sequence, increasing the activity of the ARGOS polypeptide and thereby increasing the number of tissue cells in the plant or plant part. In other embodiments, the ARGOS nucleotide construct introduced into the plant is stably incorporated into the genome of the plant.

In other methods, the number of cells and biomass of a plant tissue is inreased by increasing the level and/or activity of the ARGOS polypeptide in the plant. Such methods are disclosed in detail elsewhere herein. In one such method, an ARGOS nucleotide sequence is introduced into the plant and expression of said ARGOS nucleotide sequence decreases the activity of the ARGOS polypeptide and thereby increasing the plant growth and/or organ development in the plant or plant part. In other embodiments, the ARGOS nucleotide construct introduced into the plant is stably incorporated into the genome of the plant.

As discussed above, one of skill will recognize the appropriate promoter to use to modulate the level/activity of a plant growth and/or organ development polynucleotide and polypeptide in the plant. Exemplary promoters for this embodiment have been disclosed elsewhere herein.

Accordingly, the present disclosure further provides plants having a modified plant growth and/or organ development when compared to the plant growth and/or organ development of a control plant tissue. In one embodiment, the plant of the disclosure has an increased level/activity of the ARGOS polypeptide of the disclosure and thus has increased plant growth and/or organ development in the plant tissue. In other embodiments, the plant of the disclosure has a reduced or eliminated level of the ARGOS polypeptide of the disclosure and thus has decreased plant growth and/or organ development in the plant tissue. In other embodiments, such plants have stably incorporated into their genome a nucleic acid molecule comprising a ARGOS nucleotide sequence of the disclosure operably linked to a promoter that drives expression in the plant cell.

iv. Modulating Root Development

Methods for modulating root development in a plant are provided. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development or radial expansion.

Methods for modulating root development in a plant are provided. The methods comprise modulating the level and/or activity of the ARGOS polypeptide in the plant. In one method, an ARGOS sequence of the disclosure is provided to the plant. In another method, the ARGOS nucleotide sequence is provided by introducing into the plant a polynucleotide comprising an ARGOS nucleotide sequence of the disclosure, expressing the ARGOS sequence and thereby modifying root development. In still other methods, the ARGOS nucleotide construct introduced into the plant is stably incorporated into the genome of the plant.

In other methods, root development is modulated by altering the level or activity of the ARGOS polypeptide in the plant. An increase in ARGOS activity can result in at least one or more of the following alterations to root development, including, but not limited to, larger root meristems, increased in root growth, enhanced radial expansion, an enhanced vasculature system, increased root branching, more adventitious roots and/or an increase in fresh root weight when compared to a control plant.

As used herein, “root growth” encompasses all aspects of growth of the different parts that make up the root system at different stages of its development in both monocotyledonous and dicotyledonous plants. It is to be understood that enhanced root growth can result from enhanced growth of one or more of its parts including the primary root, lateral roots, adventitious roots, etc.

Methods of measuring such developmental alterations in the root system are known in the art. See, for example, US Patent Application Publication Number 2003/0074698 and Werner, et al., (2001) PNAS 18:10487-10492, both of which are herein incorporated by reference.

As discussed above, one of skill will recognize the appropriate promoter to use to modulate root development in the plant. Exemplary promoters for this embodiment include constitutive promoters and root-preferred promoters. Exemplary root-preferred promoters have been disclosed elsewhere herein.

Stimulating root growth and increasing root mass by increasing the activity and/or level of the ARGOS polypeptide also finds use in improving the standability of a plant. The term “resistance to lodging” or “standability” refers to the ability of a plant to fix itself to the soil. For plants with an erect or semi-erect growth habit, this term also refers to the ability to maintain an upright position under adverse (environmental) conditions. This trait relates to the size, depth and morphology of the root system. In addition, stimulating root growth and increasing root mass by increasing the level and/or activity of the ARGOS polypeptide also finds use in promoting in vitro propagation of explants.

Furthermore, higher root biomass production due to an increased level and/or activity of ARGOS activity has a direct effect on the yield and an indirect effect of production of compounds produced by root cells or transgenic root cells or cell cultures of said transgenic root cells. One example of an interesting compound produced in root cultures is shikonin, the yield of which can be advantageously enhanced by said methods.

Accordingly, the present disclosure further provides plants having modulated root development when compared to the root development of a control plant. In some embodiments, the plant of the disclosure has an increased level/activity of the ARGOS polypeptide of the disclosure and has enhanced root growth and/or root biomass. In other embodiments, such plants have stably incorporated into their genome a nucleic acid molecule comprising a ARGOS nucleotide sequence of the disclosure operably linked to a promoter that drives expression in the plant cell.

v. Modulating Shoot and Leaf Development

Methods are also provided for modulating shoot and leaf development in a plant. By “modulating shoot and/or leaf development” is intended any alteration in the development of the plant shoot and/or leaf. Such alterations in shoot and/or leaf development include, but are not limited to, alterations in shoot meristem development, in leaf number, leaf size, leaf and stem vasculature, internode length and leaf senescence. As used herein, “leaf development” and “shoot development” encompasses all aspects of growth of the different parts that make up the leaf system and the shoot system, respectively, at different stages of their development, both in monocotyledonous and dicotyledonous plants. Methods for measuring such developmental alterations in the shoot and leaf system are known in the art. See, for example, Werner, et al., (2001) PNAS 98:10487-10492 and US Patent Application Publication Number 2003/0074698, each of which is herein incorporated by reference.

The method for modulating shoot and/or leaf development in a plant comprises modulating the activity and/or level of an ARGOS polypeptide of the disclosure. In one embodiment, an ARGOS sequence of the disclosure is provided. In other embodiments, the ARGOS nucleotide sequence can be provided by introducing into the plant a polynucleotide comprising an ARGOS nucleotide sequence of the disclosure, expressing the ARGOS sequence and thereby modifying shoot and/or leaf development. In other embodiments, the ARGOS nucleotide construct introduced into the plant is stably incorporated into the genome of the plant.

In specific embodiments, shoot or leaf development is modulated by decreasing the level and/or activity of the ARGOS polypeptide in the plant. An decrease in ARGOS activity can result in at least one or more of the following alterations in shoot and/or leaf development, including, but not limited to, reduced leaf number, reduced leaf surface, reduced vascular, shorter internodes and stunted growth and retarded leaf senescence, when compared to a control plant.

As discussed above, one of skill will recognize the appropriate promoter to use to modulate shoot and leaf development of the plant. Exemplary promoters for this embodiment include constitutive promoters, shoot-preferred promoters, shoot meristem-preferred promoters and leaf-preferred promoters. Exemplary promoters have been disclosed elsewhere herein.

Decreasing ARGOS activity and/or level in a plant results in shorter internodes and stunted growth. Thus, the methods of the disclosure find use in producing dwarf plants. In addition, as discussed above, modulation of ARGOS activity in the plant modulates both root and shoot growth. Thus, the present disclosure further provides methods for altering the root/shoot ratio. Shoot or leaf development can further be modulated by decreasing the level and/or activity of the ARGOS polypeptide in the plant.

Accordingly, the present disclosure further provides plants having modulated shoot and/or leaf development when compared to a control plant. In some embodiments, the plant of the disclosure has an increased level/activity of the ARGOS polypeptide of the disclosure, altering the shoot and/or leaf development. Such alterations include, but are not limited to, increased leaf number, increased leaf surface, increased vascularity, longer internodes and increased plant stature, as well as alterations in leaf senescence, as compared to a control plant. In other embodiments, the plant of the disclosure has a decreased level/activity of the ARGOS polypeptide of the disclosure.

vi Modulating Reproductive Tissue Development

Methods for modulating reproductive tissue development are provided. In one embodiment, methods are provided to modulate floral development in a plant. By “modulating floral development” is intended any alteration in a structure of a plant's reproductive tissue as compared to a control plant in which the activity or level of the ARGOS polypeptide has not been modulated. “Modulating floral development” further includes any alteration in the timing of the development of a plant's reproductive tissue (i.e., a delayed or an accelerated timing of floral development) when compared to a control plant in which the activity or level of the ARGOS polypeptide has not been modulated. Macroscopic alterations may include changes in size, shape, number or location of reproductive organs, the developmental time period that these structures form or the ability to maintain or proceed through the flowering process in times of environmental stress. Microscopic alterations may include changes to the types or shapes of cells that make up the reproductive organs.

The method for modulating floral development in a plant comprises modulating ARGOS activity in a plant. In one method, an ARGOS sequence of the disclosure is provided. An ARGOS nucleotide sequence can be provided by introducing into the plant a polynucleotide comprising an ARGOS nucleotide sequence of the disclosure, expressing the ARGOS sequence and thereby modifying floral development. In other embodiments, the ARGOS nucleotide construct introduced into the plant is stably incorporated into the genome of the plant.

In specific methods, floral development is modulated by decreasing the level or activity of the ARGOS polypeptide in the plant. A decrease in ARGOS activity can result in at least one or more of the following alterations in floral development, including, but not limited to, retarded flowering, reduced number of flowers, partial male sterility and reduced seed set, when compared to a control plant. Inducing delayed flowering or inhibiting flowering can be used to enhance yield in forage crops such as alfalfa. Methods for measuring such developmental alterations in floral development are known in the art. See, for example, Mouradov, et al., (2002) The Plant Cell S111-S130, herein incorporated by reference.

As discussed above, one of skill will recognize the appropriate promoter to use to modulate floral development of the plant. Exemplary promoters for this embodiment include constitutive promoters, inducible promoters, shoot-preferred promoters and inflorescence-preferred promoters.

In other methods, floral development is modulated by increasing the level and/or activity of the ARGOS sequence of the disclosure. Such methods can comprise introducing an ARGOS nucleotide sequence into the plant and increasing the activity of the ARGOS polypeptide. In other methods, the ARGOS nucleotide construct introduced into the plant is stably incorporated into the genome of the plant. Increasing expression of the ARGOS sequence of the disclosure can modulate floral development during periods of stress. Such methods are described elsewhere herein. Accordingly, the present disclosure further provides plants having modulated floral development when compared to the floral development of a control plant. Compositions include plants having an increased level/activity of the ARGOS polypeptide of the disclosure and having an altered floral development. Compositions also include plants having an increased level/activity of the ARGOS polypeptide of the disclosure wherein the plant maintains or proceeds through the flowering process in times of stress.

Methods are also provided for the use of the ARGOS sequences of the disclosure to increase seed size and/or weight. The method comprises increasing the activity of the ARGOS sequences in a plant or plant part, such as the seed. An increase in seed size and/or weight comprises an increased size or weight of the seed and/or an increase in the size or weight of one or more seed part including, for example, the embryo, endosperm, seed coat, aleurone or cotyledon.

As discussed above, one of skill will recognize the appropriate promoter to use to increase seed size and/or seed weight. Exemplary promoters of this embodiment include constitutive promoters, inducible promoters, seed-preferred promoters, embryo-preferred promoters and endosperm-preferred promoters.

The method for decreasing seed size and/or seed weight in a plant comprises decreasing ARGOS activity in the plant. In one embodiment, the ARGOS nucleotide sequence can be provided by introducing into the plant a polynucleotide comprising a ARGOS nucleotide sequence of the disclosure, expressing the ARGOS sequence and thereby decreasing seed weight and/or size. In other embodiments, the ARGOS nucleotide construct introduced into the plant is stably incorporated into the genome of the plant.

It is further recognized that increasing seed size and/or weight can also be accompanied by an increase in the speed of growth of seedlings or an increase in early vigor. As used herein, the term “early vigor” refers to the ability of a plant to grow rapidly during early development, and relates to the successful establishment, after germination, of a well-developed root system and a well-developed photosynthetic apparatus. In addition, an increase in seed size and/or weight can also result in an increase in plant yield when compared to a control.

Accordingly, the present disclosure further provides plants having an increased seed weight and/or seed size when compared to a control plant. In other embodiments, plants having an increased vigor and plant yield are also provided. In some embodiments, the plant of the disclosure has an increased level/activity of the ARGOS polypeptide of the disclosure and has an increased seed weight and/or seed size. In other embodiments, such plants have stably incorporated into their genome a nucleic acid molecule comprising a ARGOS nucleotide sequence of the disclosure operably linked to a promoter that drives expression in the plant cell.

vii. Method of Use for ARGOS Promoter Polynucleotides

The polynucleotides comprising the ARGOS promoters disclosed in the present disclosure, as well as variants and fragments thereof, are useful in the genetic manipulation of any host cell, preferably plant cell, when assembled with a DNA construct such that the promoter sequence is operably linked to a nucleotide sequence comprising a polynucleotide of interest. In this manner, the ARGOS promoter polynucleotides of the disclosure are provided in expression cassettes along with a polynucleotide sequence of interest for expression in the host cell of interest. As discussed in Example 2 below, the ARGOS promoter sequences of the disclosure are expressed in a variety of tissues and thus the promoter sequences can find use in regulating the temporal and/or the spatial expression of polynucleotides of interest.

Synthetic hybrid promoter regions are known in the art. Such regions comprise upstream promoter elements of one polynucleotide operably linked to the promoter element of another polynucleotide. In an embodiment of the disclosure, heterologous sequence expression is controlled by a synthetic hybrid promoter comprising the ARGOS promoter sequences of the disclosure, or a variant or fragment thereof, operably linked to upstream promoter element(s) from a heterologous promoter. Upstream promoter elements that are involved in the plant defense system have been identified and may be used to generate a synthetic promoter. See, for example, Rushton, et al., (1998) Curr. Opin. Plant Biol. 1:311-315. Alternatively, a synthetic ARGOS promoter sequence may comprise duplications of the upstream promoter elements found within the ARGOS promoter sequences.

It is recognized that the promoter sequence of the disclosure may be used with its native ARGOS coding sequences. A DNA construct comprising the ARGOS promoter operably linked with its native ARGOS gene may be used to transform any plant of interest to bring about a desired phenotypic change, such as modulating cell number, modulating root, shoot, leaf, floral and embryo development, stress tolerance and any other phenotype described elsewhere herein.

The promoter nucleotide sequences and methods disclosed herein are useful in regulating expression of any heterologous nucleotide sequence in a host plant in order to vary the phenotype of a plant. Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

In general, methods to modify or alter the host endogenous ARGOS DNA are available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein, is generated using “custom” meganucleases produced to modify plant genomes (see e.g., WO 2009/114321; Gao, et al., (2010) Plant Journal 1:176-187). Another site-directed engineering is through the use of zinc finger domain recognition coupled with the restriction properties of restriction enzyme. See e.g., Urnov, et al., (2010) Nat Rev Genet. 11(9):636-46; Shukla, et al., (2009) Nature 459(7245):437-41. A transcription activator-like (TAL) effector-DNA modifying enzyme (TALE or TALEN) is also used to engineer changes in plant genome. See e.g., US Patent Application Publication Number 2011/0145940, Cermak, et al., (2011) Nucleic Acids Res. 39(12) and Boch, et al., (2009) Science 326(5959):1509-12.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases and those involved in housekeeping, such as heat shock proteins. More specific categories of transgenes, for example, include genes encoding important traits for agronomics, insect resistance, disease resistance, herbicide resistance, sterility, grain characteristics and commercial products. Genes of interest include, generally, those involved in oil, starch, carbohydrate or nutrient metabolism as well as those affecting kernel size, sucrose loading, and the like.

In certain embodiments the nucleic acid sequences of the present disclosure can be used in combination (“stacked”) with other polynucleotide sequences of interest in order to create plants with a desired phenotype. The combinations generated can include multiple copies of any one or more of the polynucleotides of interest. The polynucleotides of the present disclosure may be stacked with any gene or combination of genes to produce plants with a variety of desired trait combinations, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802 and 5,703,409); barley high lysine (Williamson, et al., (1987) Eur. J. Biochem. 165:99-106 and WO 1998/20122) and high methionine proteins (Pedersen, et al., (1986) J. Biol. Chem. 261:6279; Kirihara, et al., (1988) Gene 71:359 and Musumura, et al., (1989) Plant Mol. Biol. 12:123); increased digestibility (e.g., modified storage proteins (U.S. patent application Ser. No. 10/053,410, filed Nov. 7, 2001) and thioredoxins (U.S. patent application Ser. No. 10/005,429, filed Dec. 3, 2001)), the disclosures of which are herein incorporated by reference. The polynucleotides of the present disclosure can also be stacked with traits desirable for insect, disease or herbicide resistance (e.g., Bacillus thuringiensis toxic proteins (U.S. Pat. Nos. 5,366,892; 5,747,450; 5,737,514; 5723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene) and glyphosate resistance (EPSPS gene)) and traits desirable for processing or process products such as high oil (e.g., U.S. Pat. No. 6,232,529); modified oils (e.g., fatty acid desaturase genes (U.S. Pat. No. 5,952,544; WO 1994/11516)); modified starches (e.g., ADPG pyrophosphorylases (AGPase), starch synthases (SS), starch branching enzymes (SBE) and starch debranching enzymes (SDBE)) and polymers or bioplastics (e.g., U.S. Pat. No. 5.602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present disclosure with polynucleotides affecting agronomic traits such as male sterility (e.g., see, U.S. Pat. No. 5.583,210), stalk strength, flowering time or transformation technology traits such as cell cycle regulation or gene targeting (e.g., WO 1999/61619; WO 2000/17364; WO 1999/25821), the disclosures of which are herein incorporated by reference.

In one embodiment, sequences of interest improve plant growth and/or crop yields. For example, sequences of interest include agronomically important genes that result in improved primary or lateral root systems. Such genes include, but are not limited to, nutrient/water transporters and growth induces. Examples of such genes, include but are not limited to, maize plasma membrane H⁺-ATPase (MHA2) (Frias, et al., (1996) Plant Cell 8:1533-44); AKT1, a component of the potassium uptake apparatus in Arabidopsis, (Spalding, et al., (1999) J Gen Physiol 113:909-18); RML genes which activate cell division cycle in the root apical cells (Cheng, et al., (1995) Plant Physiol 108:881); maize glutamine synthetase genes (Sukanya, et al., (1994) Plant Mol Biol 26:1935-46) and hemoglobin (Duff, et al., (1997) J. Biol. Chem 27:16749-16752, Arredondo-Peter, et al., (1997) Plant Physiol. 115:1259-1266; Arredondo-Peter, et al., (1997) Plant Physiol 114:493-500 and references sited therein). The sequence of interest may also be useful in expressing antisense nucleotide sequences of genes that that negatively affects root development.

Additional, agronomically important traits such as oil, starch and protein content can be genetically altered in addition to using traditional breeding methods. Modifications include increasing content of oleic acid, saturated and unsaturated oils, increasing levels of lysine and sulfur, providing essential amino acids and also modification of starch. Hordothionin protein modifications are described in U.S. Pat. Nos. 5,703,049, 5,885,801, 5,885,802 and 5,990,389, herein incorporated by reference. Another example is lysine and/or sulfur rich seed protein encoded by the soybean 2S albumin described in U.S. Pat. No. 5,850,016 and the chymotrypsin inhibitor from barley, described in Williamson, et al., (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. patent application Ser. No. 08/740,682, filed Nov. 1, 1996, and WO 1998/20133, the disclosures of which are herein incorporated by reference. Other proteins include methionine-rich plant proteins such as from sunflower seed (Lilley, et al., (1989) Proceedings of the World Congress on Vegetable Protein Utilization in Human Foods and Animal Feedstuffs, ed. Applewhite (American Oil Chemists Society, Champaign, Ill.), pp. 497-502, herein incorporated by reference); corn (Pedersen, et al., (1986) J. Biol. Chem. 261:6279; Kirihara, et al., (1988) Gene 71:359, both of which are herein incorporated by reference) and rice (Musumura, et al., (1989) Plant Mol. Biol. 12:123, herein incorporated by reference). Other agronomically important genes encode latex, Floury 2, growth factors, seed storage factors and transcription factors.

Insect resistance genes may encode resistance to pests that have great yield drag such as rootworm, cutworm, European Corn Borer and the like. Such genes include, for example, Bacillus thuringiensis toxic protein genes (U.S. Pat. Nos. 5,366,892; 5,747,450; 5,736,514; 5,723,756; 5,593,881 and Geiser, et al., (1986) Gene 48:109), and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432; and Mindrinos, et al., (1994) Cell 78:1089), and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), or other such genes known in the art. The bar gene encodes resistance to the herbicide basta, the nptII gene encodes resistance to the antibiotics kanamycin and geneticin and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids and levels of cellulose. In corn, modified hordothionin proteins are described in U.S. Pat. Nos. 5,703,049, 5,885,801, 5,885,802 and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones and the like. The level of proteins, particularly modified proteins having improved amino acid distribution to improve the nutrient value of the plant, can be increased. This is achieved by the expression of such proteins having enhanced amino acid content.

This disclosure can be better understood by reference to the following non-limiting examples. It will be appreciated by those skilled in the art that other embodiments of the disclosure may be practiced without departing from the spirit and the scope of the disclosure as herein disclosed and claimed.

### EXAMPLES

### Example 1

### Isolation of ARGOS Sequences

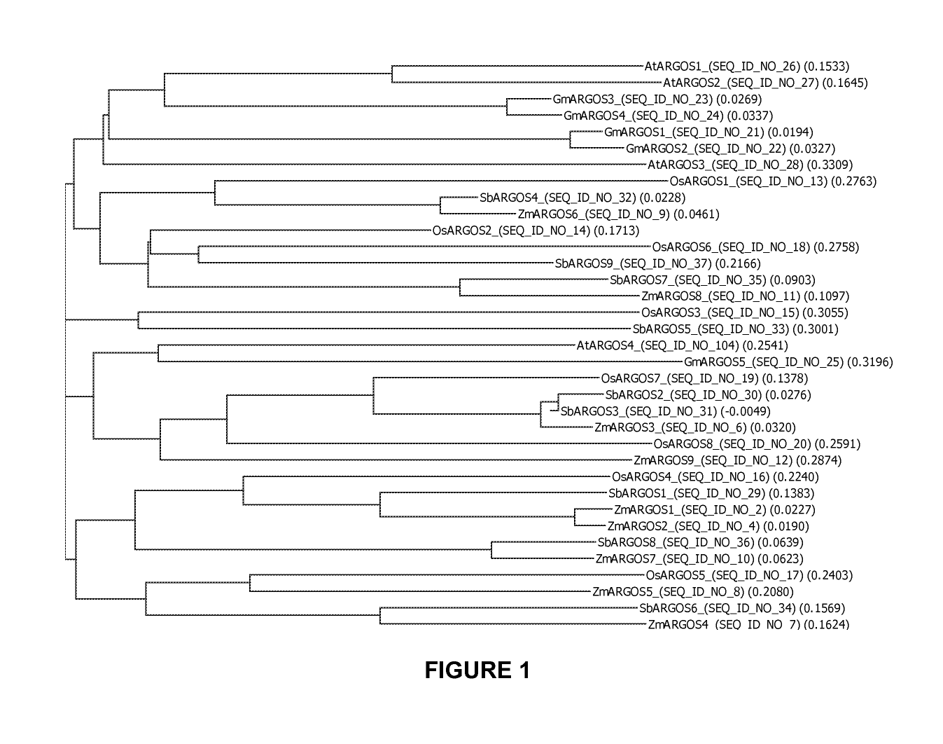

A routine for identifying all members of a gene family was employed to search for the ARGOS genes of interest. A diverse set of all the members of the gene family as protein sequences was prepared. This data includes sequences from other species. These species are searched against a proprietary maize sequence dataset and a nonredundant set of overlapping hits is identified. Separately, one takes the nucleotide sequences of any genes of interest in hand and searches against the database and a nonredundant set of all overlapping hits are retrieved. The set of protein hits are then compared to the nucleotide hits. If the gene family is complete, all of the protein hits are contained within the nucleotide hits. The ARGOS family of genes consists of 3 Arabidopsis genes, 8 rice genes, 9 maize genes, 9 sorghum genes and 5 soybean genes. A dendrogram representation of the interrelationship of the proteins encoded by these genes is provided as FIG. 1.

### Example 2

### ARGOS Sequence Analysis

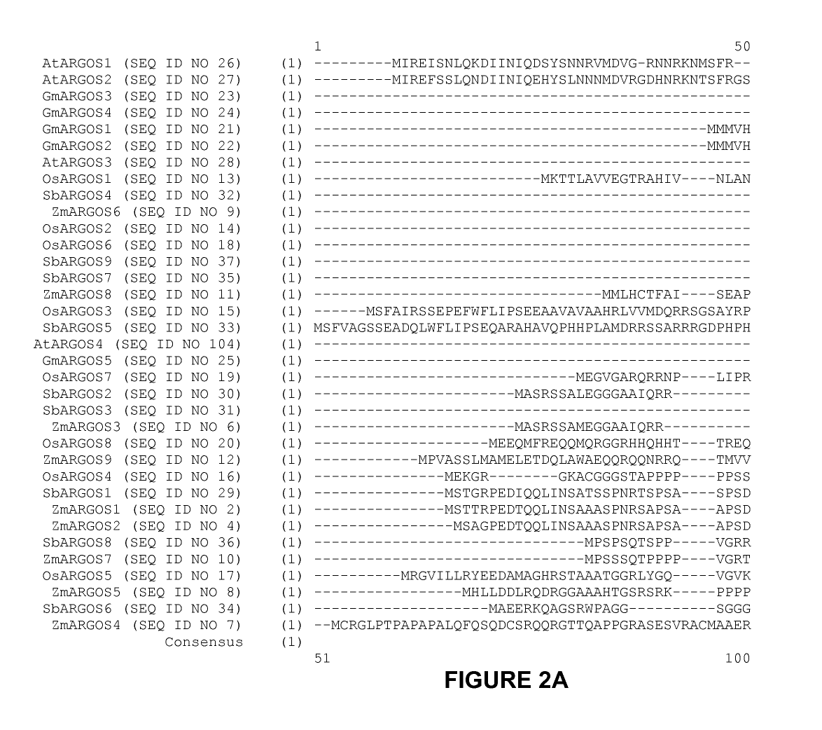

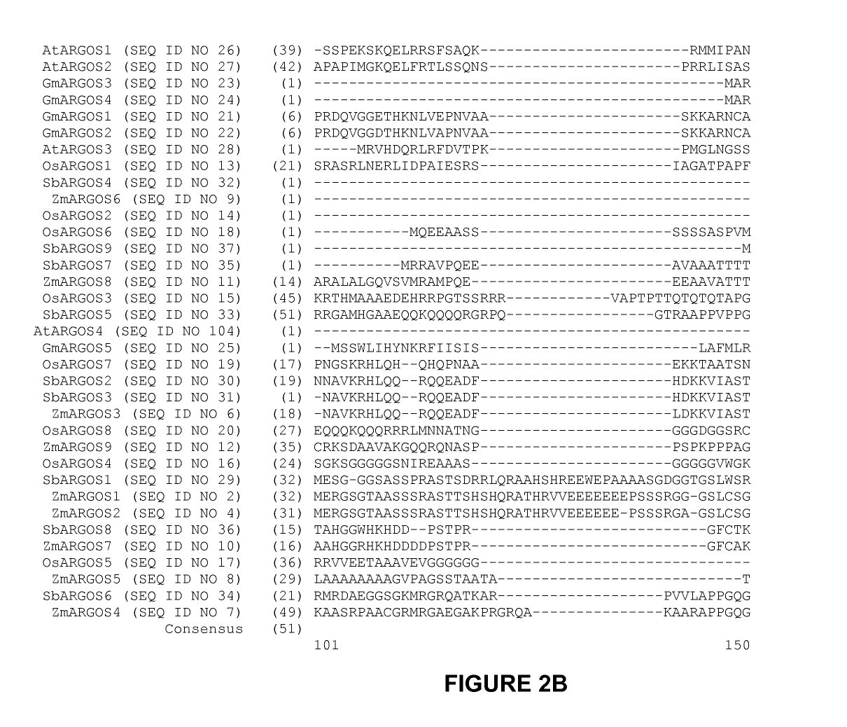

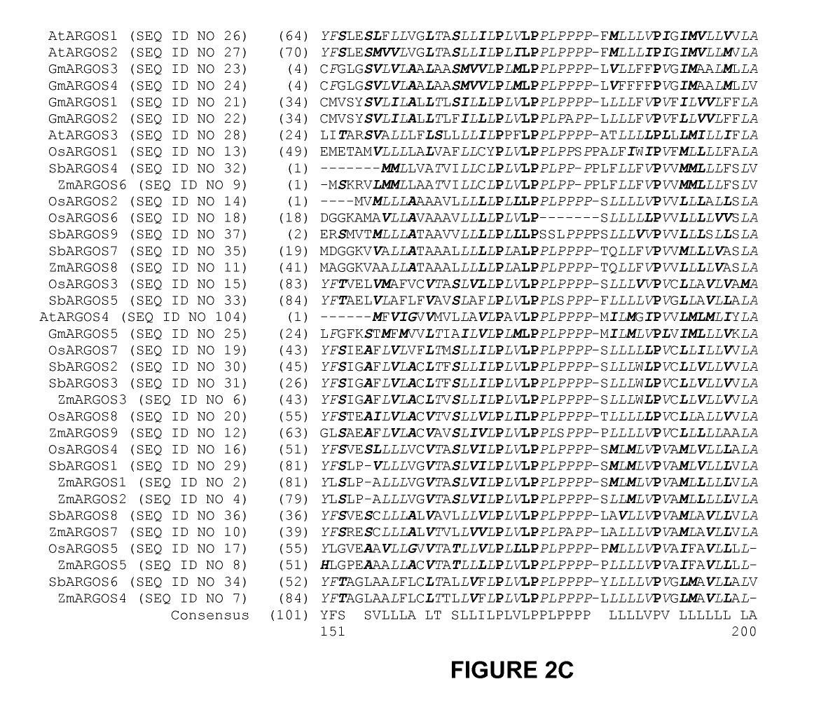

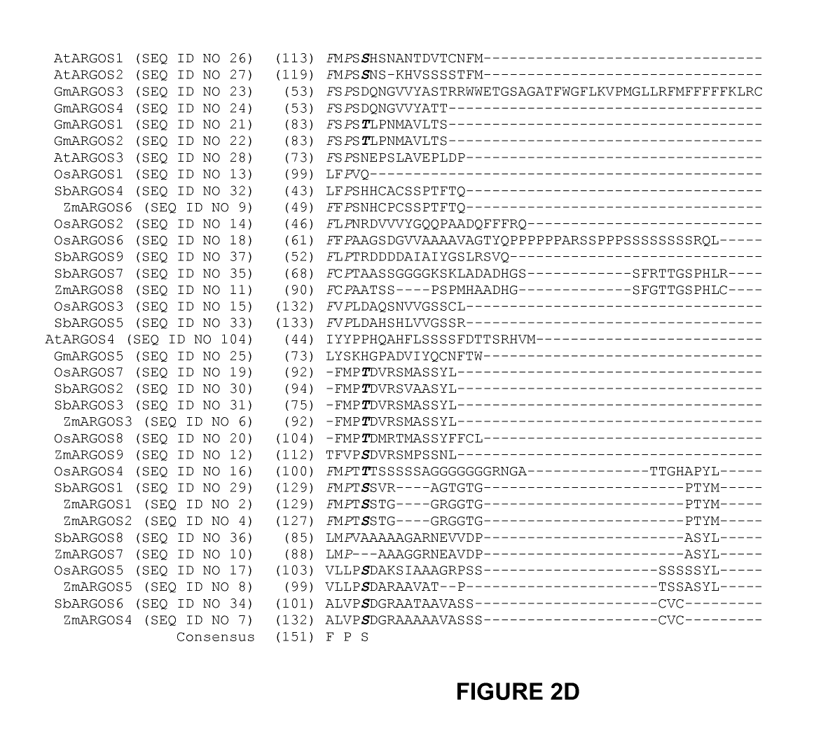

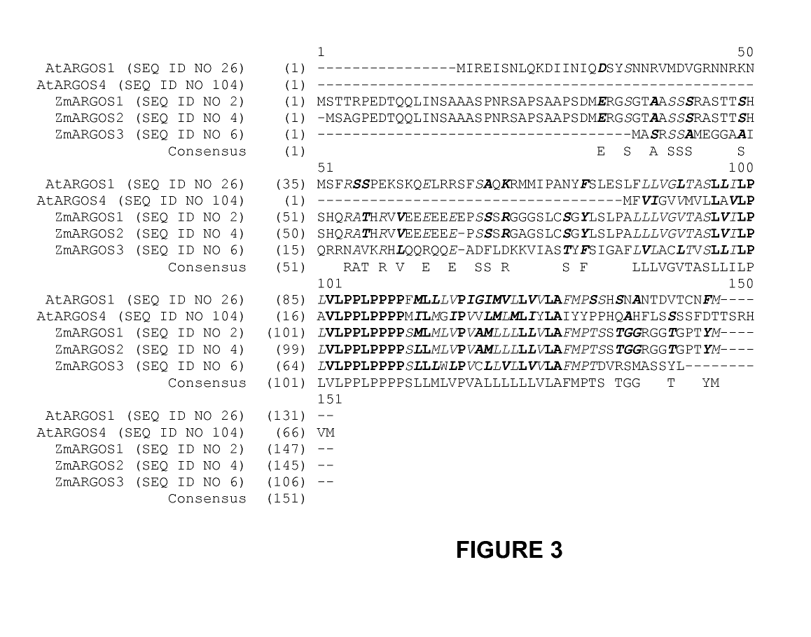

The ZmARGOS polypeptides of the current disclosure have common characteristics with ARGOS genes in a variety of plant species. The relationship between the genes of the multiple plant species is shown in an alignment, see, FIG. 2. FIG. 3 contains ZmARGOS1, 2, 3 and AtARGOS1 (SEQ ID NOS: 2, 4, 6 and 26). The proteins encoded by the ARGOS genes have a well-conserved proline rich region near the C-terminus. The N-termini are more divergent. The proteins are relatively short, averaging 110 amino acids.

### Example 3

### Transformation and Regeneration of Transgenic Plants

Immature maize embryos from greenhouse donor plants are bombarded with a plasmid containing the ZmARGOS sequence operably linked to the drought-inducible promoter RAB17 promoter (Vilardell, et al., (1990) Plant Mol Biol 14:423-432) and the selectable marker gene PAT, which confers resistance to the herbicide Bialaphos. Alternatively, the selectable marker gene is provided on a separate plasmid. Transformation is performed as follows. Media recipes follow below.

Preparation of Target Tissue

The ears are husked and surface sterilized in 30% Clorox® bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

A plasmid vector comprising the ARGOS sequence operably linked to an ubiquitin promoter is made. This plasmid DNA plus plasmid DNA containing a PAT selectable marker is precipitated onto 1.1 pm (average diameter) tungsten pellets using a CaCl₂ precipitation procedure as follows:

100 μl prepared tungsten particles in water

10 μl (1 μg) DNA in Tris EDTA buffer (1 μg total DNA)

100 μl 2.5 M CaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for increased drought tolerance. Assays to measure improved drought tolerance are routine in the art and include, for example, increased kernel-earring capacity yields under drought conditions when compared to control maize plants under identical environmental conditions. Alternatively, the transformed plants can be monitored for a modulation in meristem development (i.e., a decrease in spikelet formation on the ear). See, for example, Bruce, et al., (2002) Journal of Experimental Botany 53:1-13.

Bombardment and Culture Media

Bombardment medium (560Y) comprises 4.0 g/I N6 basal salts (SIGMA C-1416), 1.0 ml/l Eriksson's Vitamin Mix (1000X SIGMA-1511), 0.5 mg/l thiamine HCl, 120.0 g/l sucrose, 1.0 mg/l 2,4-D and 2.88 g/l L-proline (brought to volume with D-I H₂O following adjustment to pH 5.8 with KOH); 2.0 g/l Gelrite® (added after bringing to volume with D-I H₂O) and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000X SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite® (added after bringing to volume with D-I H₂O) and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite® (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto™-agar (added after bringing to volume with polished D-I H₂O), sterilized and cooled to 60° C.

### Example 4

### Agrobacterium-Mediated Transformation

For Agrobacterium-mediated transformation of maize with an antisense sequence of the ZmARGOS sequence of the present disclosure, preferably the method of Zhao is employed (U.S. Pat. No. 5,981,840 and PCT Patent Publication Number WO 1998/32326, the contents of which are hereby incorporated by reference). Briefly, immature embryos are isolated from maize and the embryos contacted with a suspension of Agrobacterium, where the bacteria are capable of transferring the ARGOS sequence to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step), and preferably calli grown on selective medium are cultured on solid medium to regenerate the plants. Plants are monitored and scored for a modulation in meristem development. For instance, alterations of size and appearance of the shoot and floral meristems and/or increased yields of leaves, flowers and/or fruits.

### Example 5

### Over Expression of ZmARGOS Affects Plant Size and Organ Size

The function of the ZmARGOS gene was tested by using transgenic plants expressing the Ubi-ZmARGOS transgene. Transgene expression was confirmed by using transgene-specific primer RT-PCR (SEQ ID NO: 38 for ARGOS and SEQ ID NO: 39 for PIN). T1 plants from nine single-copy events were evaluated in the field. Transgenic plants showed positive growth enhancements in several aspects.

Vegetative Growth and Biomass Accumulation

Compared to the non transgenic sibs, the transgenic plants (in T1 generation) showed an average of 4% increase in plant height across all 9 events and up to 12% in the highest event. The stem of the transgenic plants was thicker than the non transgenic siblings as measured by stem diameter values with an average of 9% to 22% increase among the nine events. The increase of the plant height and the stem thickness resulted in a larger plant stature and biomass for the transgenic plants. Estimated biomass accumulation showed an increase of 30% on average and up to 57% in transgenic positive lines compared to the negative siblings.

ZmARGOS was found to impact plant growth mainly through accelerating the growth rate but not extending the growth period. The enhanced growth, i.e., increased plant size and biomass accumulation, appears to be largely due to an accelerated growth rate and not due to an extended period of growth because the transgenic plants were not delayed in flowering based on the silking and anthesis dates. In fact, the transgenic plants flowered earlier than the non-transgenic siblings. On average across the events, the days to flowering was shortened to between 30 heat units (1-1.5 days), and 69 heat units (2-2.5 days). Therefore, overexpressing of the ZmARGOS gene accelerated the growth rate of the plant. Accelerated growth rate appears to be associated with an increased cell proliferation rate.

The enhanced vegetative growth, biomass accumulation in transgenics and accelerated growth rate were further tested with extensive field experiments in both hybrid and inbred backgrounds at advanced generation (T3). Transgenic plants reproducibly showed increased plant height up to 18%, stem diameter up to 10%, stalk dry mass up to 15%, increased leaf area up to 14%, total plant dry mass up to 25%. Earlier flowering observed in T1 generation was again observed in T3 generation.

Reproductive Growth and Grain Yield

Overexpression of the ZmARGOS1 gene also enhanced the reproductive organ growth. T1 Transgenic plants showed increased ear length, about 10% on the average of nine events, and up to 14% for the highest event. Total kernel weight per ear increased 13% on average and up to 70% for one event. The increase in total kernel weight appears to be attributed to the increased kernel numbers per ear and kernel size. The average of the nine events showed that the kernel number per ear increased 8%, and up to 50% in the highest event. The 100-kernel weight increased 5% on average, and up to 13% for the highest event. The positive change in kernel and ear characteristics is associated with grain yield increase.

The enhanced reproductive growth and grain yield of transgenics was again confirmed in extensive field experiments at the advanced generation (T3). The enhancement was observed in both inbred and hybrid backgrounds. As compared to the non-transgenic sibs as controls, the transgenic plants showed a significantly increase in primary ear dry mass up to 60%, secondary ear dry mass up to 4.7 folds, tassel dry mass up to 25% and husk dry mass up to 40%. The transgenics showed up to 13% increase in kernel number per ear, and up to 13% grain yield increase.

Transgenic plants also showed reduced ASI, up to 40 heat units, reduced barrenness up to 50% and reduced number of aborted kernels up to 64%. The reduction is more when the plants were grown at a high plant density stressed condition. A reduced measurement of these parameters is often related to tolerance to biotic stress.

In addition, transgene expression level is significantly correlated with the ear dry mass.

### Example 6

### T1 Assay Results for the UBIZM-ARGOS—Field Study Results

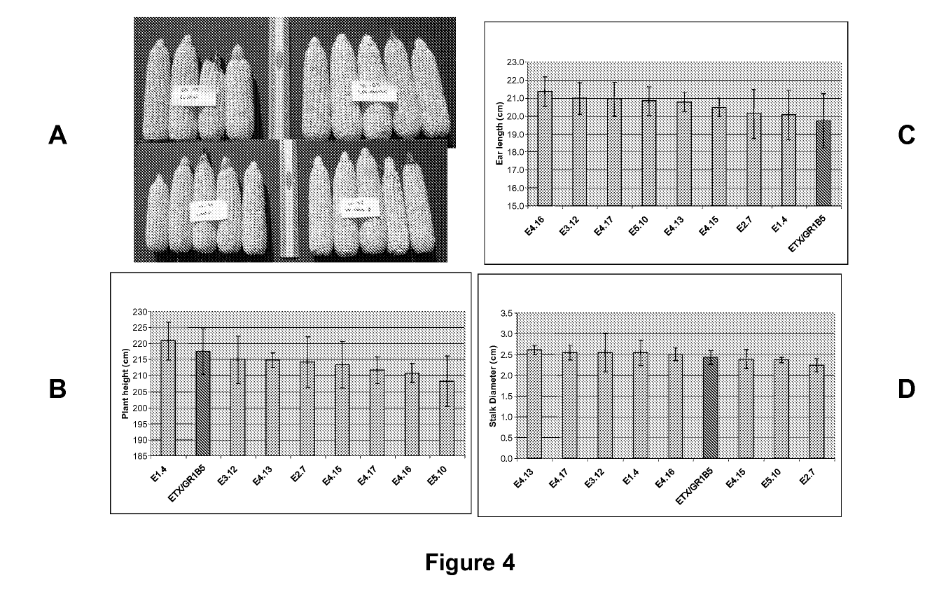

ZmARGOS8 showed overall positive effects on yield with no particular patterns of interaction with environments and no significant negative interaction or significant yield reduction in any of the environments. Therefore, it was chosen for extended yield testing in the following year under drought stress and nitrogen fertilizer application treatments for its potential under drought and low nitrogen stress. The transgenic hybrid showed overall yield advantage under these treatments without any significant yield reduction in any particular environments (FIG. 4). ZmARGOS8 exhibited positive effects in multiple environments from multiple years' yield trials, and did not show any negative interaction with particular environments. ZmARGOS8 actually not only gave a yield advantage in “normal” conditions, but also under limited N application and limited water supply or drought stressed conditions.

### Example 7

### Comparision of ARGOS 1 and 8 and Secondary Structure

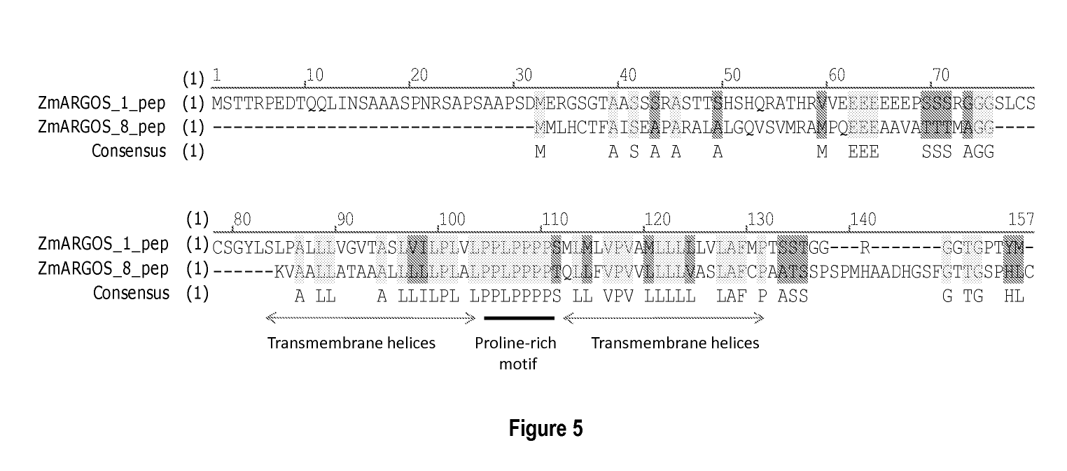

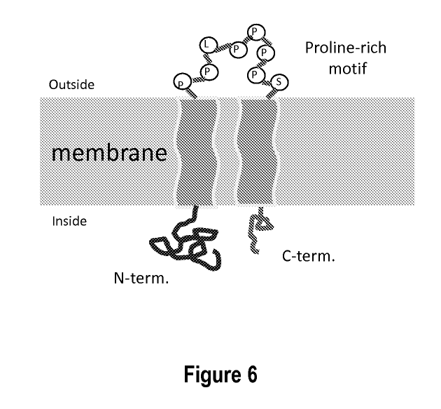

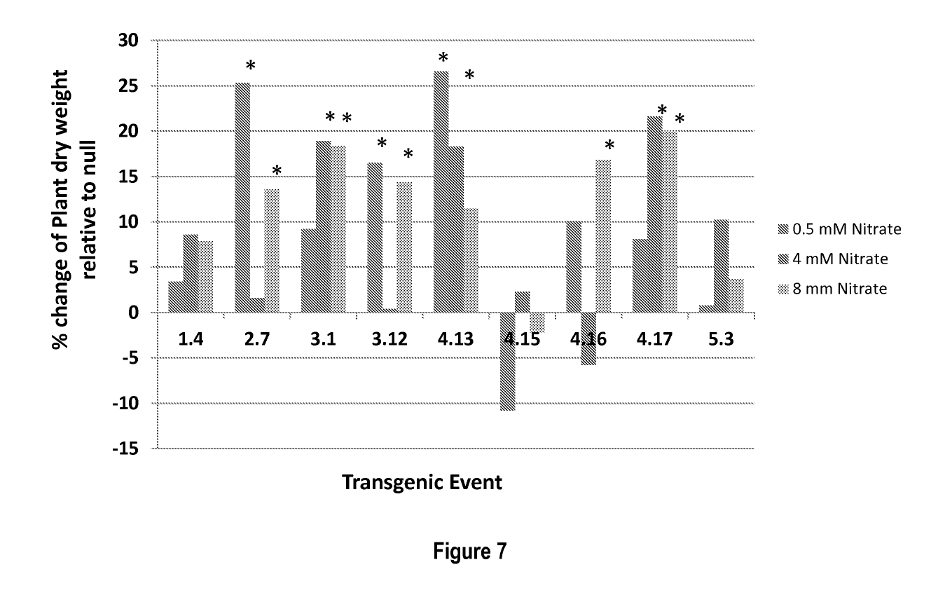

Maize ARGOS8 shows overall 24.8% identify with ZmARGOS1 at amino acid sequence (FIG. 5), but the proline-rich motif and the two transmembrane helices are highly conserved between ZmARGOS8 and ZmARGOS1. In the proline-rich motif, 7 out of 8 amino acids are identical between ZmARGOS1 and ZmARGOS8. The only amino acid difference in this motif is a Ser to Thr, which is considered a conservative amino acid change as both are hydroxyl containing amino acids. The ZmARGOS8 shows a similar predicted protein structure as the ZmARGOS 1 although their overall identity is low (FIG. 6).

### Example 8

### Biomass Accumulation Under Multiple Nitrogen Concentrations

Expression of ZmARGOS8 under a maize constitutive ubiquitin promoter enhanced plant growth at seedling stage in elite maize hybrid. Total 10 transgenic and 10 non-transgenic null plants each from 9 transgenic maize events were grown randomly at 0.5 mM, 4 mM, and 8 mM nitrate concentrations in Turface® for 3 weeks in greenhouse. Plants were harvested and plant dry weight (DWT) was determined. Three out of 9 events tested showed a significant increase in plant dry weight compared to null in 2 mM and 4 mM nitrate concentrations. At 8 mM high nitrate concentration, 5 out of 9 events showed a significant increase in plant dry weight. For example, Event 4.17 showed a 21.6% and 20.1% increase in dry weight at 4 mM nitrate and 8 mM nitrate concentrations respectively (FIG. 7).

### Example 9

### Field Trials Under Normal Nitrogen

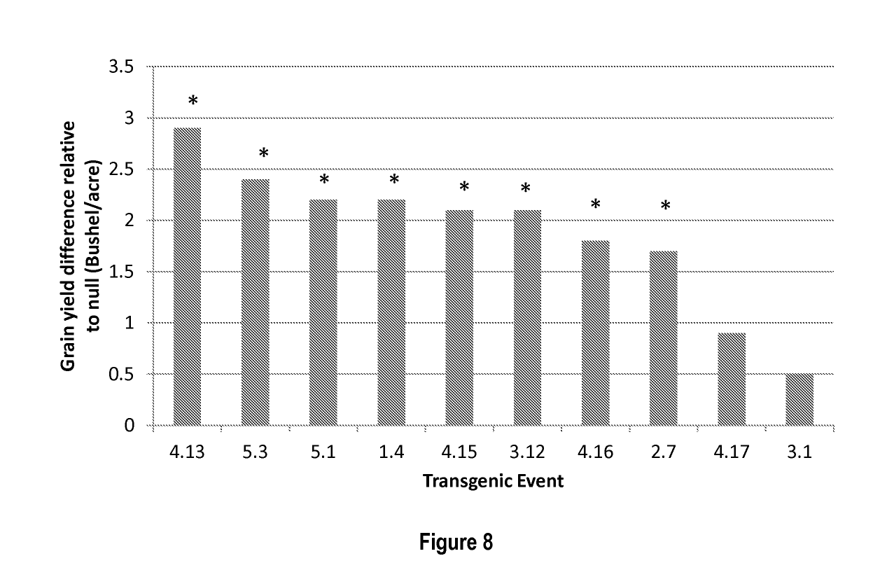

Those events were further tested first in field at 4 normal nitrogen locations in the Midwestern United States with 4 replicates per location. Later, the field tests were expanded to 3 normal nitrogen locations with 4-6 replicates per location, 3 low nitrogen locations with 6 replicates per location and 2 drought locations with 4-6 replicates. Two year multiple location analysis indicated that 8 out of 10 events showed a significant increase in grain yield across the drought, low N and normal N environments at p<0.1. The best event showed an average 2.9 bushel per acre yield advantage over control (FIG. 8).

### Example 10

### FastCorn Yield Component Analysis

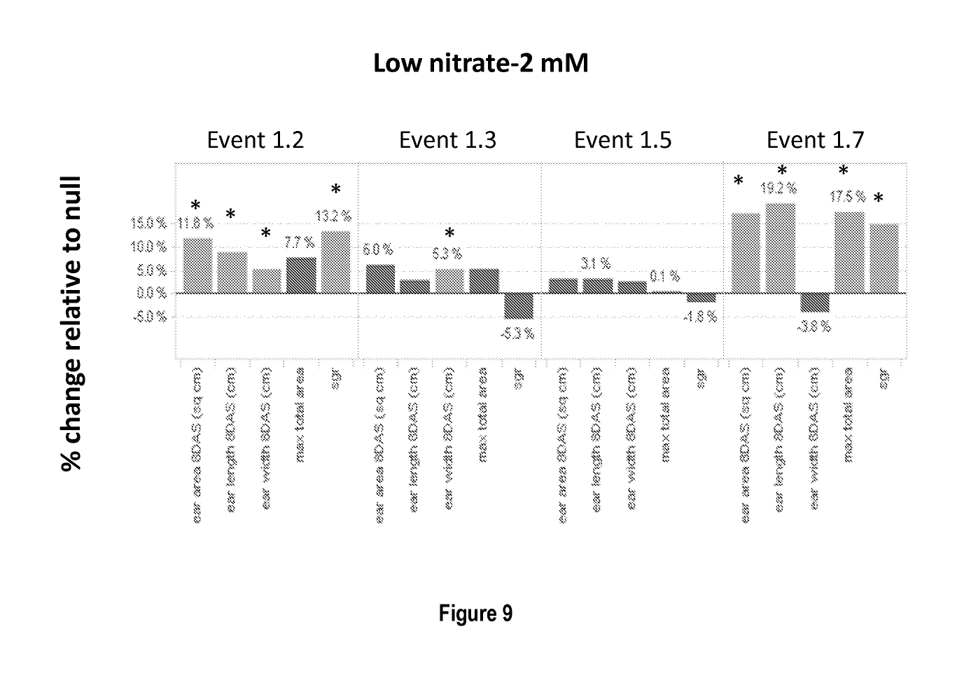

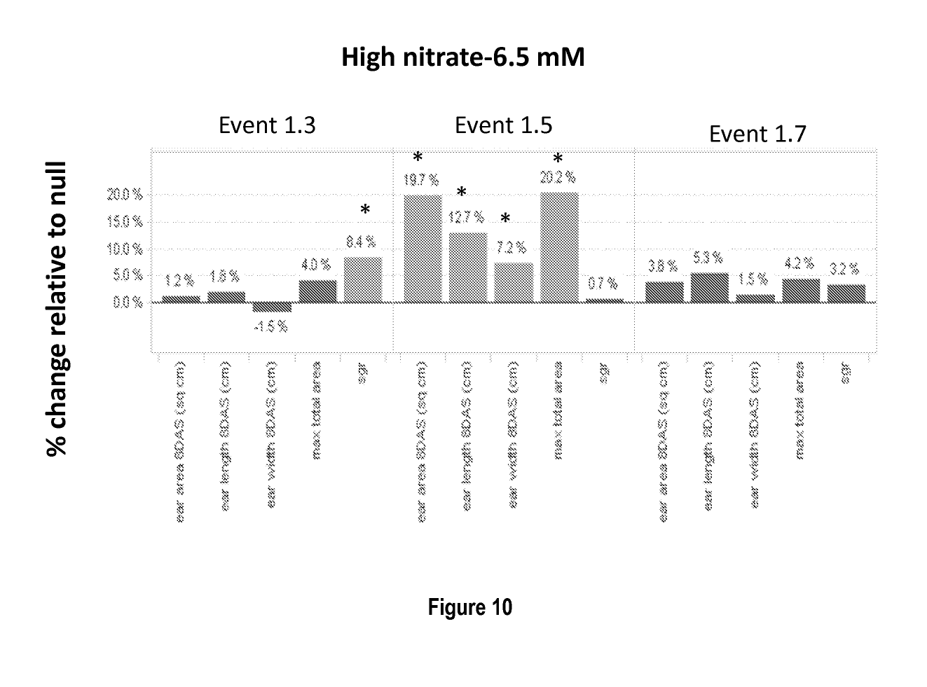

To understand the impact of ZmARGOS8 on yield components, Ubi:ZmARGOS8 construct was re-transformed into a fast cycle maize germplasm, GS3XGaspe. Total 15 transgenic T1 plant and 15 null segregants from 3-4 events were grown in an automated greenhouse under 2 mM nitrate and 6.5 mM nitrate concentrations. Plant relative growth rate (sgr) and max total area were determined by image technology. Ear length, width and area were determined at 8 days after silking using ear photometry. Under 2 mM nitrate, two out of 4 events showed a significant increase in ear length, ear area and relative growth rate at p<0.05. Under 6.5 mM nitrate, one out of 3 events showed a significant increase in ear length, ear area, ear width and max total area at p<0.05 (FIG. 9 and FIG. 10).

### Example 11

### Overexpression of ARGOS1 Reduces Ethylene Responses in Maize

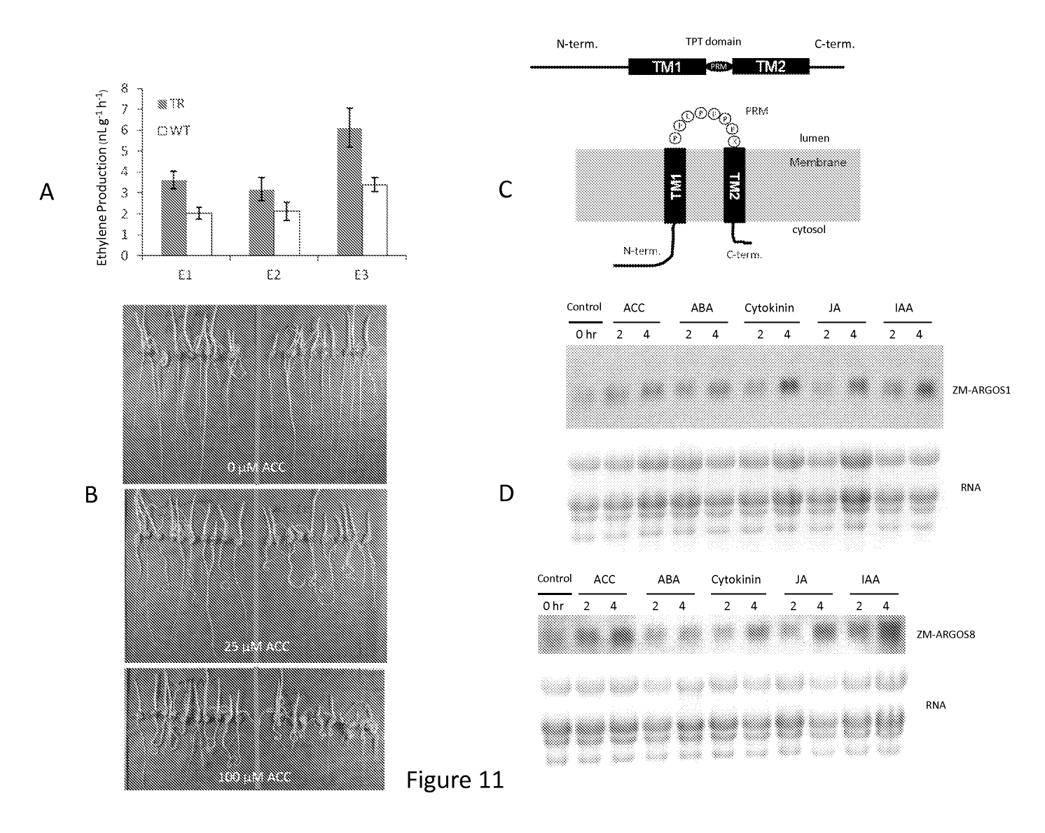

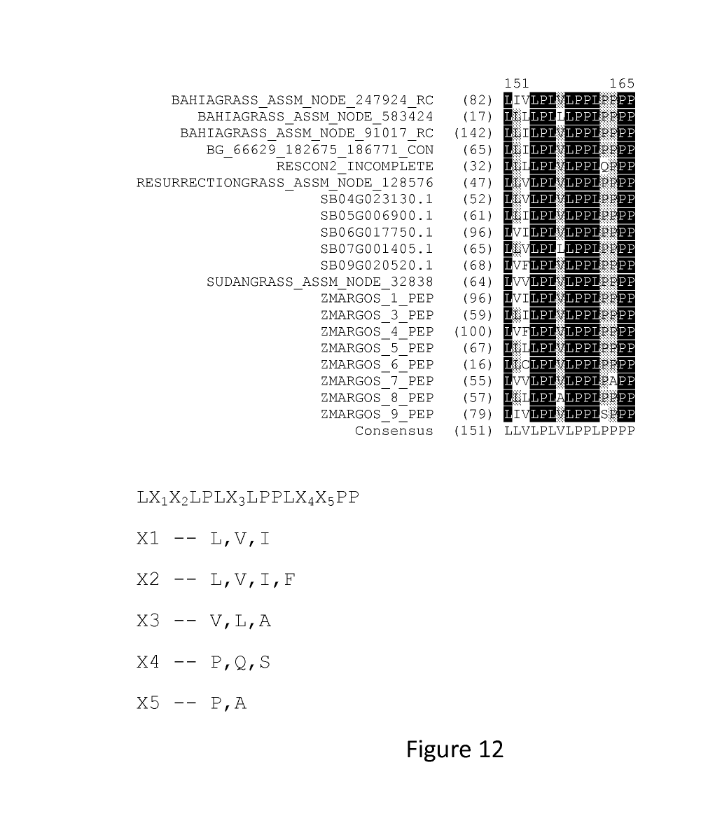

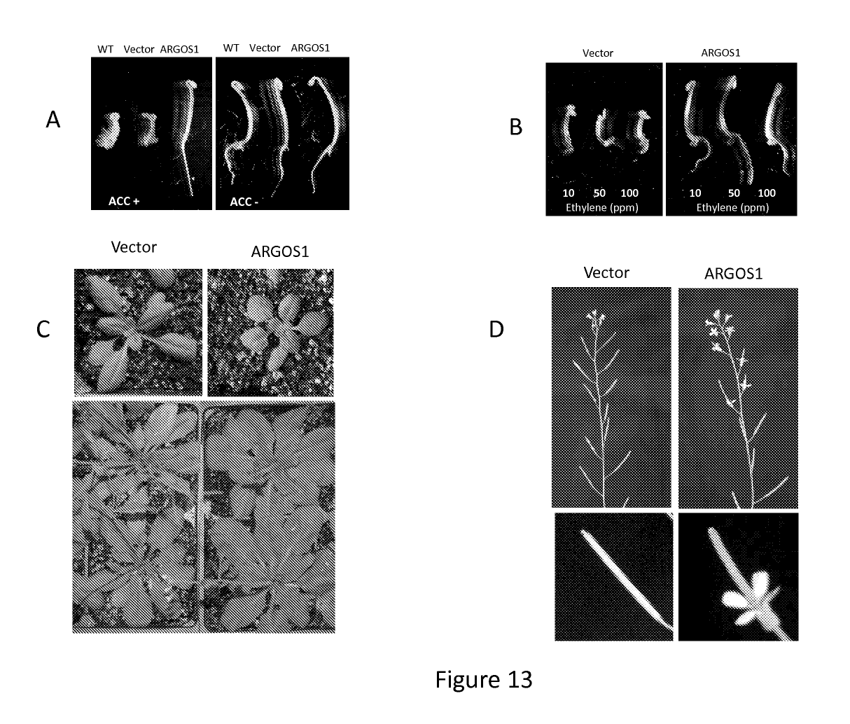

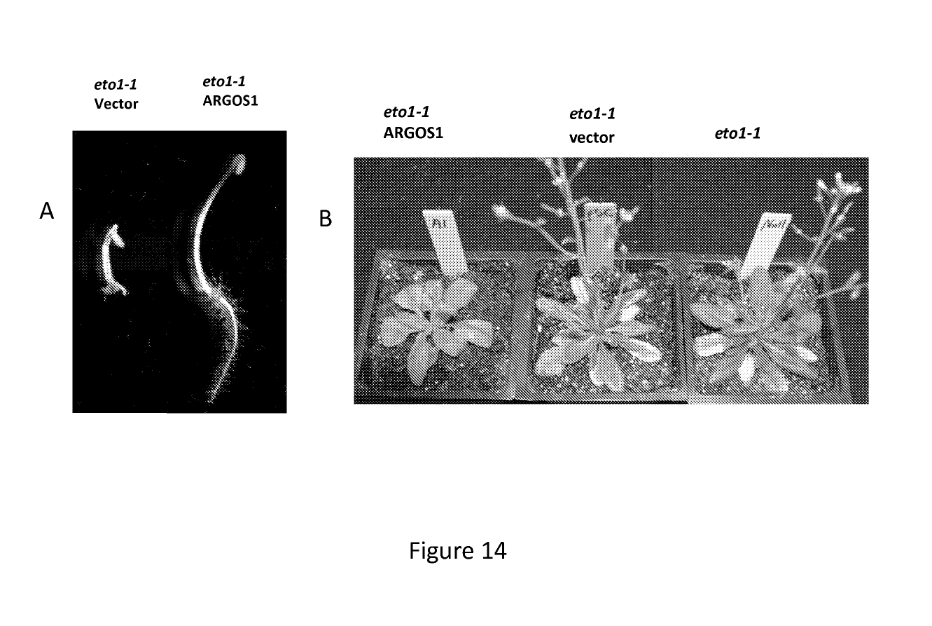

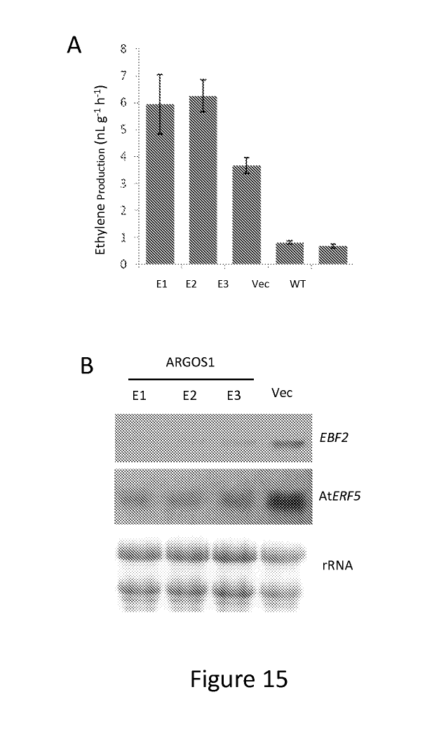

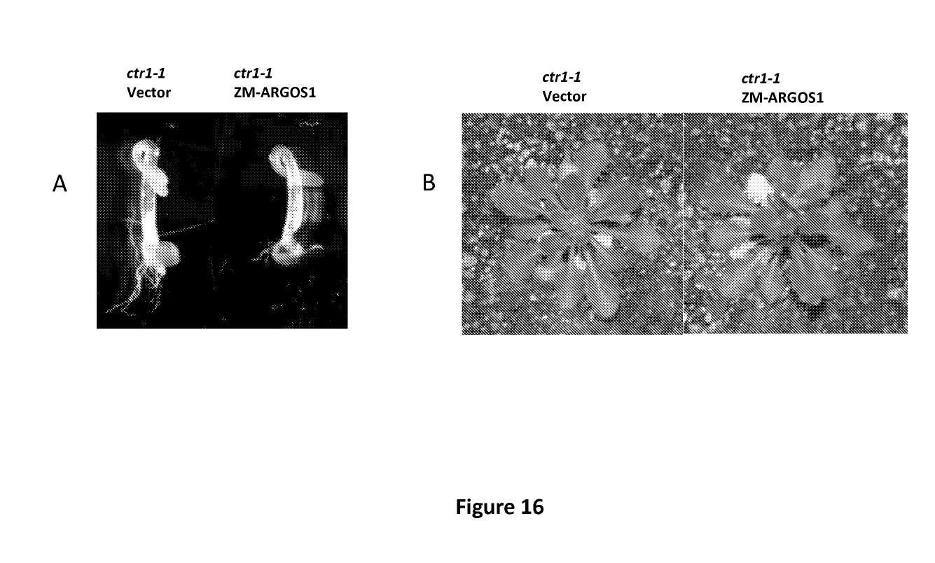

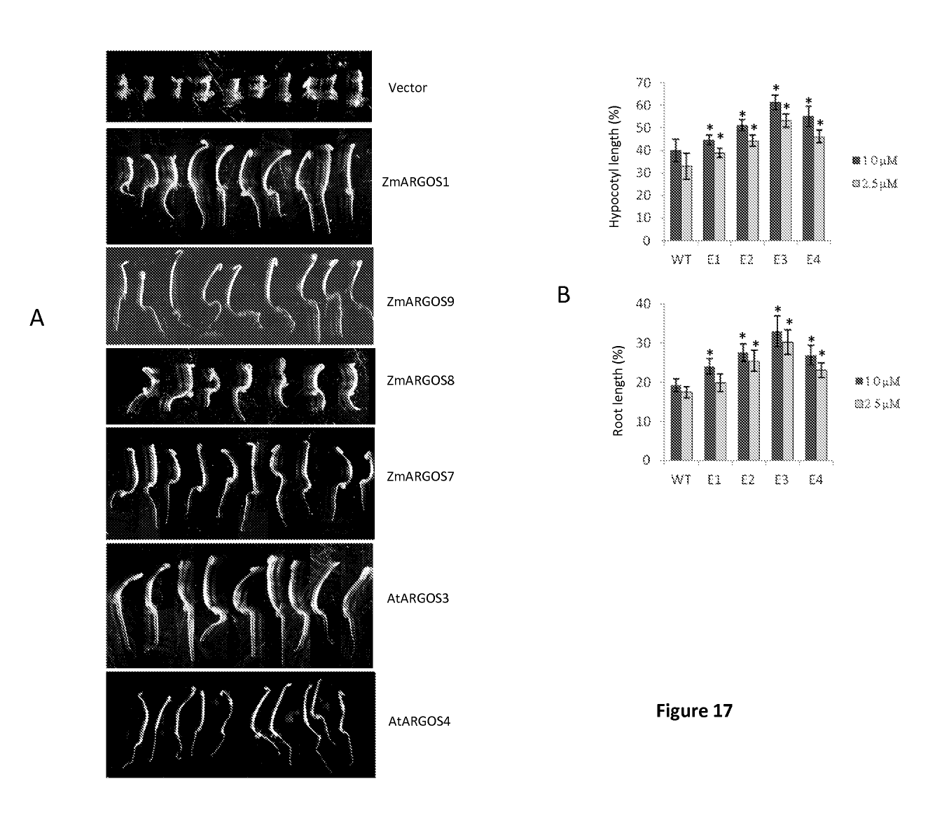

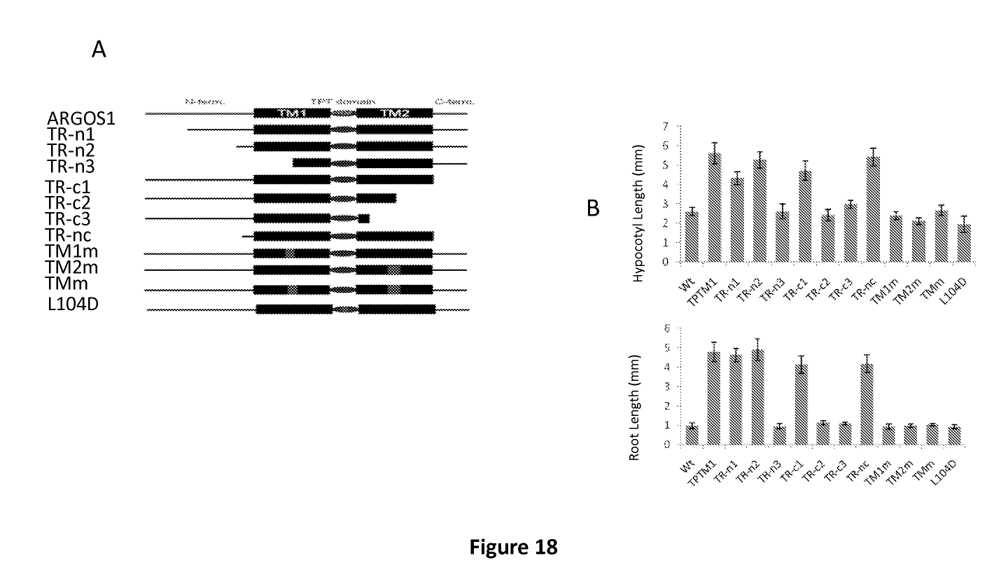

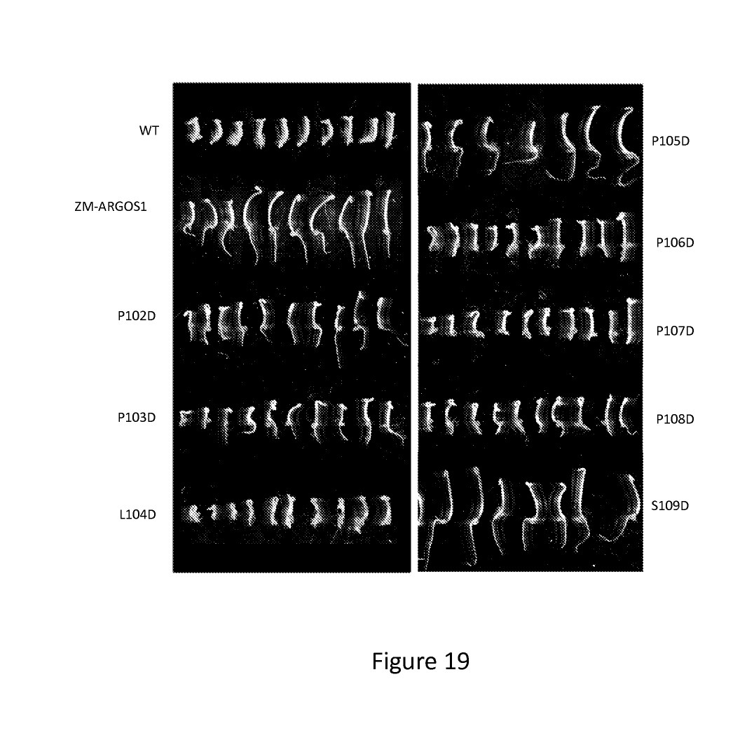

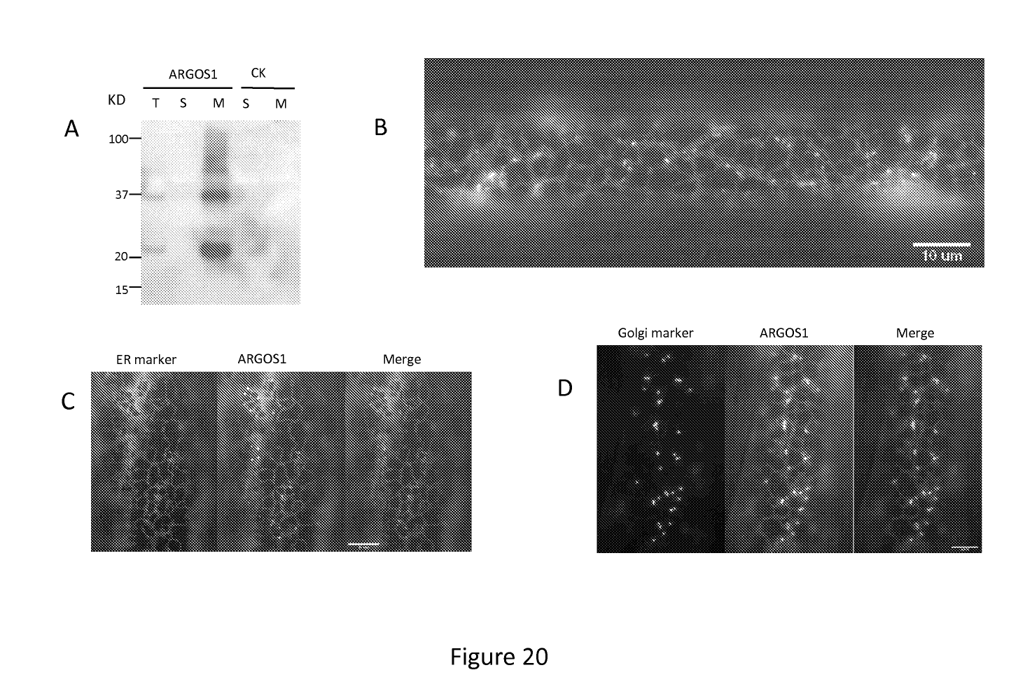

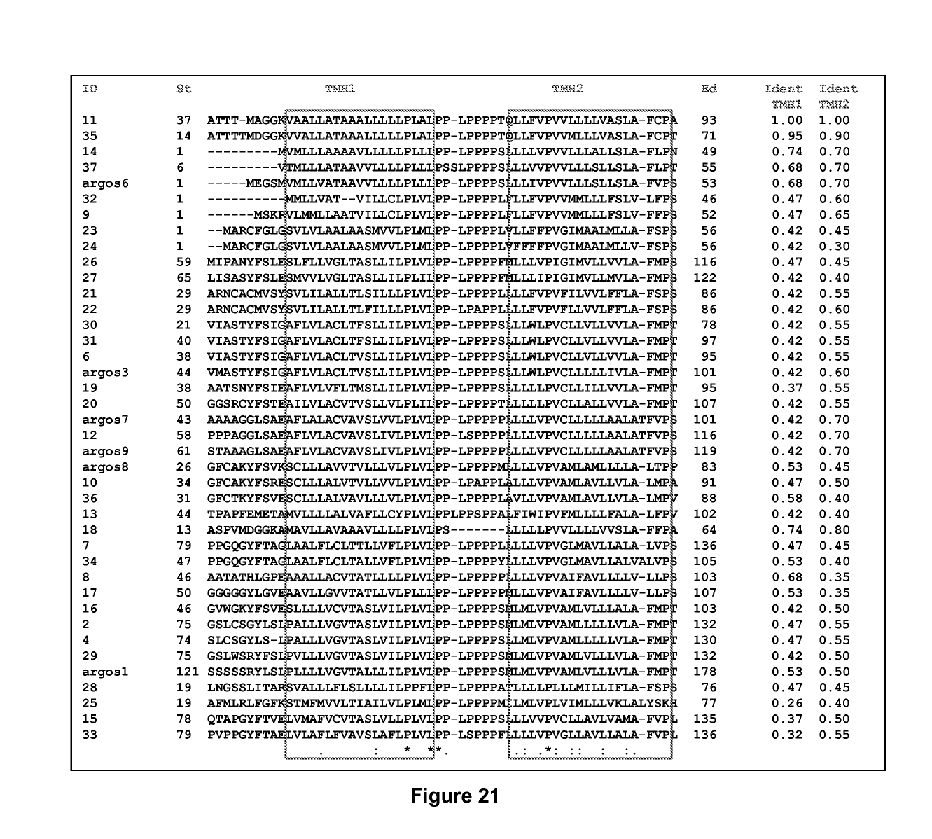

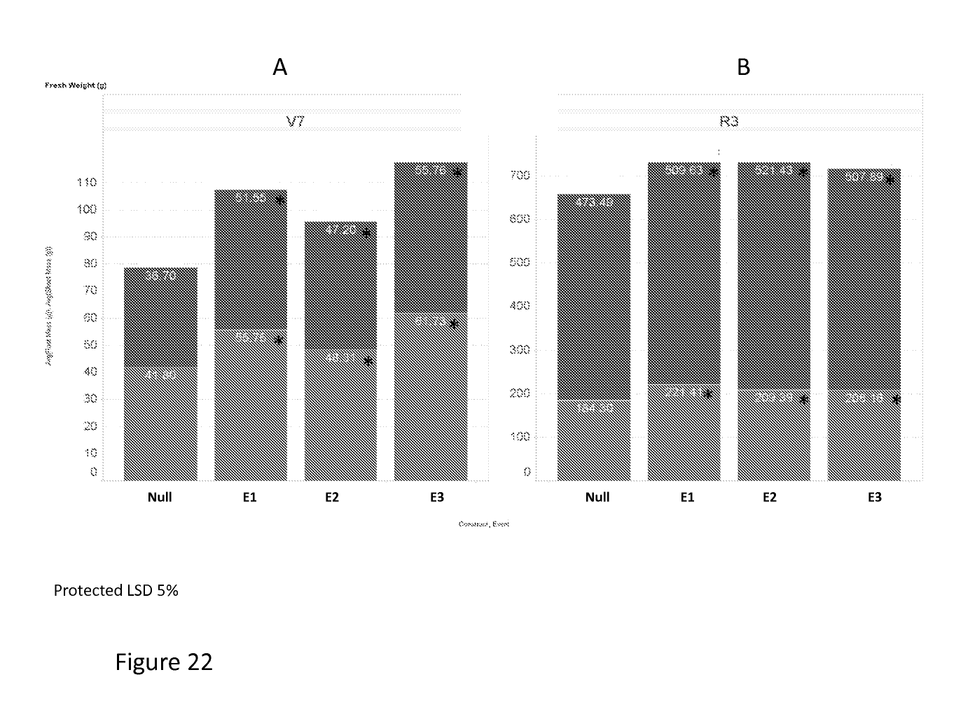

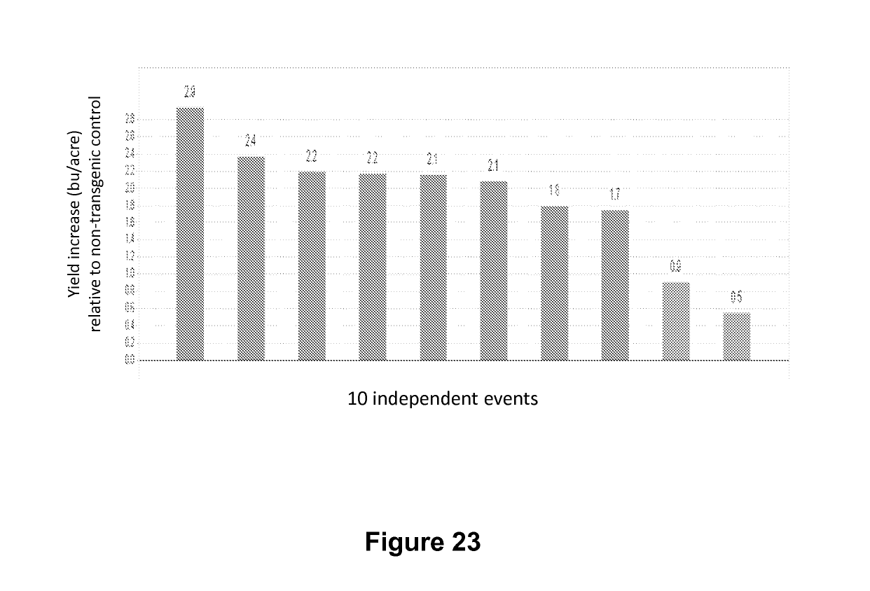

To identify candidate genes that could be used to improve maize productivity, genes were systematically overexpressed in maize under the control of the maize ubiquitin 1 (Ubi) promoter. In addition, the levels of phytohormones in transgenic events were determined. Transgenic plants overexpressing a maize ARGOS gene were found to produce 50-80% more ethylene than the wild-type segregants (FIG. 11A). The response of the transgenic plants to exogenously supplied ethylene was further investigated. Treatments with the ethylene precursor ACC reduced root elongation and affected root gravitropism in non-transgenic seedlings, but to a lesser extent in transgenic events (FIG. 11B). The inhibition of root growth was detectable at 25 αM ACC and the severity of the phenotype intensified with an increase in ACC concentration. In the absence of exogenously supplied ACC, no difference in seedling growth was detected between transgenic and non-transgenic seedlings. The enhanced ethylene biosynthesis and reduced ethylene response in the transgenic plants indicate that overexpression of the gene may affect ethylene sensitivity in maize plants.

### Example 12

### Analysis of ARGOS1 Structure

The maize ARGOS1 (SEQ ID NO: 4) encodes a small protein of 144 amino acid residues. Sequence hydropathy analysis predicted two transmembrane alpha-helices, TM1 (aa79-101) (SEQ ID NO: 90) and TM2 (aa110-134) (SEQ ID NO: 91) (FIG. 11C). The peptide segment connecting TM1 and TM2 consists of eight amino acids, six of which are proline (FIG. 11C). Therefore, the loop region (aa102-109, PPLPPPPS) is referred to as proline-rich motif (PRM) (SEQ ID NO: 88). The N- and C-terminal regions were predicted to reside on the cytoplasm side of a membrane and the PRM loop on the lumen side (FIG. 11C). BLAST searches revealed seven genes in the maize genome encoding proteins that also contain the TM1-PRM-TM2 (TPT) domain (SEQ ID NO: 89). The PRM sequence is almost identical among the maize proteins and the transmembrane helices have a high percentage of identical or similar amino acids (FIG. 12). Expression of ARGOS1 gene was elevated in maize seedlings that were treated with IAA, cytokinin and jasmonic acid (FIG. 11D). The IAA, ACC, cytokinin and jasmonic acid treatment also increased the transcript levels of ARGOS8 (FIG. 11D).

Maize ARGOS1 and Arabidopsis ARGOS1 share 36% amino acid sequence identity. The expression of ANT homologous genes in the Ubi:ARGOS1 maize was examined using qRT-PCR, but no significant difference in expression was observed between the transgenic and wild-type maize plants.

### Example 13

### Ectopic Expression of Maize ARGOS1 Confers Ethylene Insensitivity in Arabidopsis

To further investigate the effect of ARGOS on plant responses to ethylene, the maize ARGOS1 gene was ectopically expressed in Arabidopsis under the control of the cauliflower mosaic virus (CaMV) 35S promoter. Thirty-six events were selected based on the expression of the yellow florescence protein (YFP) and bialaphos resistance (BAR) selection marker genes. The expression of ZmARGOS1 in Arabidopsis was confirmed by Northern blotting analysis of ten events (data not shown). Arabidopsis seeds were germinated in the dark in the presence or absence of gaseous ethylene or ACC. Etiolated seedlings of wild-type Col-0 plants showed inhibition of hypocotyl and root growth, exaggerated curvature of the apical hook and excessive radical swelling of the hypocotyl (FIG. 13A and 13B), which is the typical triple response of Arabidopsis to ethylene exposure (Guzman and Ecker, 1990). Transgenic seedlings generated from the empty vector control had the same ethylene response phenotype as the wild-type Col-0. However, the etiolated 35S:ZmARGOS1 seedlings displayed elongated roots and hypocotyls in the presence of ethylene or ACC (FIGS. 13A and 13B). The ethylene response of exaggerated tightening of the apical hook and swelling of the hypocotyl exhibited in wild-type plants were absent in the 35S:ARGOS1 seedlings. A consistent phenotype was observed when ACC concentrations were increased to 50 μM (data not shown). These results demonstrate that 35S:ZmARGOS1 transgenic Arabidopsis plants are insensitive to exogenous ethylene.

The 35S:ZmARGOS1 plants grew more slowly than controls under conditions of 16-h light period (approximate 120 mE m⁻² s⁻¹) at 24° C. and 8-h dark period at 23° C. The rosette diameter was smaller and expanding leaves were wider, but shorter (FIG. 13C upper). Flowering was delayed anywhere from 3-10 days (FIG. 13C lower). By bolting time, rosette leaves, however, were wider and longer in the 35S:ZmARGOS1 plants than controls due to longer growth duration. In the wild-type Col-0, the floral organs, such as petals, sepals and stamens abscised soon after pollination and inflorescences generally had three to five opened flowers. In contrast, petals and sepals of the 35S:ZmARGOS1 plants remained turgid and intact for a long time and abscission of the perianth organs were delayed. As a consequence, the inflorescences had about 10 opened flowers (FIG. 13D). The mature transgenic plants also exhibited delayed leaf senescence (FIG. 13C). The phenotypes of the 35S:ZmARGOS1 seedlings and adult plants are typical of the ethylene insensitive mutants.

To confirm that transgenic plants are insensitive to endogenous ethylene, the ethylene over-production mutant etol-1 was transformed with 35S:ZmARGOS1. Etiolated seedlings of the etol-1 mutant exhibited the phenotype of constitutive ethylene responses in the absence of exogenous ethylene (FIG. 14A), as expected (Chae, et al., 2003; Guzman and Ecker, 1990). The light-grown plants had dark green leaves and flowered earlier than wild-type plants. Rosette leaves in mature plants senesced early. Overexpression of ZmARGOS1 abolished the constitutive ethylene response phenotype of the etol-1 seedlings grown in the dark (FIG. 14A). Rosette leaves of the light-grown 35S:ZmARGOS1 plants had greater leaf surface than the etol-1 mutant at bolting time. Flowering and rosette leaf senescence were delayed in the 35S:ZmARGOS1-eto1-1 plants (FIG. 14B). This phenotype is similar to that of 35S:ZmARGOS1 in the wild-type background. This genetic analysis demonstrated that the 35S:ZmARGOS1 plant is insensitive to ethylene.

### Example 14

### Ethylene Biosynthesis is Increased, But the Expression of Ethylene Responsive Genes is Down-Regulated in the ZmARGOS1 Arabidopsis Plants

Because ethylene biosynthesis is enhanced in ethylene insensitive Arabidopsis mutants (Guzman and Ecker, 1990), ethylene evolution in the 35S:ZmARGOS1 plants was measured.

The transgenic leaves released 5 to 7-fold more ethylene than the vector control and wild-type plants (FIG. 15A), demonstrating increased ethylene biosynthesis activity in Arabidopsis overexpressing the ZmARGOS1.

To seek additional molecular evidence for ethylene insensitivity conferred by ARGOS1, expression of ethylene-regulated genes was investigated. Because of increased ethylene biosynthesis in the 35S ZmARGOS1 plants, one would predict that expression of ethylene responsive genes would be induced should the transgenic plant have sensed ethylene normally. Expression of Arabidopsis EIN3-BINDING F-BOX2 (EBF2) is regulated by the EIN3 transcription factor and the transcript level of EBF2 is reduced in ethylene insensitive mutants, such as ein2, ein3 and ein6. Northern analysis showed that the steady-state level of mRNA for EBF2 was down-regulated in the 35S:ARGOS1 plant relative to the control (FIG. 15B and Table 2). Arabidopsis ERF5 is an ethylene responsive-element binding factor (ERF) inducible by ethylene. In the 35S:ARGOS1 plants, the expression of AtERF5 was reduced in comparison to the vector control (FIG. 15B and Table 2). Expression levels of other ERF genes in 19-day-old aerial tissues (rosette leaves and apical meristem) of the 35S:ARGOS1 plants was measured and vector controls using RNA-Seq. The transcript levels of eleven ERF genes were found down-regulated at least 50% in the 35S:ARGOS1 plant relative to the vector control (Table 2). Among the ERF genes, AtERF1, 2, 4, 5, 9, 11, 72 and ERF1 (At3g23240) are inducible by ethylene. AtERF3 is not responsive to ethylene treatments (Fujimoto, et al., 2000) and it was determined that the expression of AtERF3 was not changed in the 35S:ARGOS1 plant in comparison to the vector control (Table 2). As predicted, the expression of the ERF-regulated plant defensin genes was also reduced in the ARGOS1 transgenic plants (Table 2). Another group of ethylene inducible genes are EDF1/TEM1, EDF2/RAV2, EDF3 and EDF4/RAV1. Three of them were down-regulated in the 35S:ARGOS1 plants (Table 2). These results confirmed that the 35S:ARGOS1 plants were unable to properly sense endogenous ethylene and suggested that ARGOS1 may act on the ethylene signaling components upstream of EIN3.

Table 2 shows the effects of overexpressing TPTM1 on expression of ethylene responsive genes, flowering genes and leaf senescence genes in Arabidopsis. RNA was extracted from aerial tissues of 19-day-old Arabidopsis plants before bolting. RNA-Seq was performed to quantify gene expression using Illumina technology. Sequence reads were bowtie aligned to Arabidopsis gene set and normalized to relative parts per kilobase per ten million (RPKtM). Values are mean±standard deviation, three replications for transgenics and four replications for vector controls. TR, 35S:TPTM1 transgenic plants; Ve: vector controls. p: t-test statistic (two-sided) p-value; PermQ: permutation false discovery rate q-value.

The quantification of transcriptome also revealed that the expression of the floral repressor FLOWERING LOCUS C (FLC) and MADS AFFECTING FLOWERING 5 (MAF5) was up-regulated in the 35S:ARGOS1 transgenic plant while the transcript levels of the floral integrator SUPPRESSOR OF OVEREXPRESSIONOFCONSTANS1 (SOC1) and LEAFY (LFY) and the floral meristem identity gene APETALA1 (AP1), AP3 and AGAMOUS were down-regulated (Table 2). The expression pattern is in agreement with the delayed floral transition phenotype displayed in the 35S:ARGOS1 plants. Enhanced FLC and reduced SOC1, FLOWERING LOCUS T (FT) and AP1 expression have been reported in the ethylene insensitive mutant etr1, ein2-1 and ein3-1. In addition, the ethylene inducible NAC transcription factor AtNAC2/ORE1/ANAC092 and AtNAPIANACO29 were significantly suppressed in the 35S:ARGOS1 transgenic plants relative to controls (Table 2). AtNAC2 is a central regulator of age-dependent senescence in Arabidopsis and its expression in roots is down-regulated in the ethylene insensitive mutant etr1 and ein2-1 and up-regulated in ethylene over-production mutant etol-1 (He et al., 2005). AtNAP also plays an import role in leaf senescence (Guo and Gan, 2006). The reduced AtNAC2 and AtANP expression in the ARGOS1 plants is consistent with the delayed leaf senescence phenotype.

TABLE 2
Gene Expression Profile

| | | TR | Ve | TR/Ve | t-test | |
|---|---|---|---|---|---|---|
| Gene | Locus | (RPKtM) | (RPKtM) | Ratio | p | PermQ |
| AtERF1 | At4g17500 | 112.5 ± 8.7 | 211.3 ± 13.2 | 0.53 | 0.0001 | 0.0239 |
| AtERF2 | At5g47220 | 186.1 ± 8.8 | 347.9 ± 24.2 | 0.53 | 0.0000 | 0.0193 |
| AtERF3 | At1g50640 | 481.9 ± 14.4 | 478.0 ± 19.2 | 1.01 | 0.7744 | 0.9096 |
| AtERF4 | At3g15210 | 419.7 ± 19.9 | 649.9 ± 31.5 | 0.65 | 0.0001 | 0.0241 |
| AtERF5 | At5g47230 | 69.4 ± 4.6 | 270.5 ± 33.0 | 0.26 | 0.0000 | 0.0105 |
| AtERF6 | At4g17490 | 88.7 ± 10.2 | 236.9 ± 17.0 | 0.37 | 0.0000 | 0.0176 |
| AtERF9 | At5g44210 | 17.4 ± 4.9 | 53.9 ± 11.9 | 0.32 | 0.0019 | 0.0736 |
| AtERF11 | At1g28370 | 30.2 ± 4.2 | 74.9 ± 13.6 | 0.40 | 0.0010 | 0.0555 |
| AtERF13 | At2g44840 | 11.7 ± 5.8 | 26.4 ± 7.4 | 0.45 | 0.0524 | 0.2816 |
| AtERF72 | At3g16770 | 1079.2 ± 196.3 | 2541.1 ± 263.7 | 0.42 | 0.0004 | 0.0447 |
| AtERF104 | At5g61600 | 233.6 ± 8.6 | 556.1 ± 50.1 | 0.42 | 0.0000 | 0.0120 |
| ERF1 | At3g23240 | 2.5 ± 0.3 | 5.2 ± 3.2 | 0.48 | 0.2969 | 0.6048 |
| PDF1.2 | At5g44420 | 147.7 ± 51.5 | 564.9 ± 77.7 | 0.26 | 0.0009 | 0.0553 |
| PDF1.2c | At5g44430 | 31.7 ± 15.1 | 222.0 ± 43.5 | 0.14 | 0.0005 | 0.0460 |
| PDF1.2b | At2g26020 | 26.1 ± 8.8 | 209.8 ± 26.8 | 0.12 | 0.0001 | 0.0236 |
| Chitinase | At2g43590 | 52.6 ± 9.3 | 127.5 ± 40.8 | 0.41 | 0.0109 | 0.1497 |
| CHI-B | At3g12500 | 37.2 ± 5.7 | 57.8 ± 11.8 | 0.64 | 0.0376 | 0.2466 |
| PR4 | At3g04720 | 779.0 ± 44.8 | 1175.1 ± 117.0 | 0.66 | 0.0014 | 0.0625 |
| EBF2 | At5g25350 | 305.8 ± 25.2 | 737.8 ± 43.0 | 0.41 | 0.0000 | 0.0105 |
| EBF1 | At2g25490 | 871.3 ± 14.4 | 824.8 ± 49.0 | 1.06 | 0.1733 | 0.4703 |
| EDF1 | At1g25560 | 416.5 ± 29.7 | 733.3 ± 37.6 | 0.57 | 0.0001 | 0.0205 |
| EDF2 | At1g68840 | 490.3 ± 34.8 | 1200.1 ± 36.0 | 0.41 | 0.0000 | 0.0064 |
| EDF3 | At3g25730 | 51.0 ± 13.2 | 36.8 ± 11.8 | 1.39 | 0.1640 | 0.4605 |
| EDF4 | At1g13260 | 795.6 ± 15.8 | 1339.5 ± 34.6 | 0.59 | 0.0000 | 0.0034 |
| FLC | At5g10140 | 15.5 ± 2.7 | 2.8 ± 2.3 | 5.62 | 0.0138 | 0.1653 |
| MAF5 | At5g10140 | 121.1 ± 21.1 | 13.0 ± 4.8 | 9.33 | 0.0003 | 0.0388 |
| SOC1 | At2g45660 | 749.5 ± 13.7 | 1019.7 ± 36.0 | 0.74 | 0.0000 | 0.0183 |
| LFY | At5g61850 | 1.5 ± 0.9 | 4.2 ± 1.6 | 0.35 | 0.0296 | 0.2248 |
| FT | At1g65480 | 7.3 ± 8.7 | 21.0 ± 7.8 | 0.35 | 0.1143 | 0.3913 |
| AP1 | At1g69120 | 5.4 ± 0.5 | 25.0 ± 7.9 | 0.22 | 0.0004 | 0.0430 |
| AP3 | At3g54340 | 2.5 ± 1.4 | 12.6 ± 5.1 | 0.20 | 0.0118 | 0.1542 |
| AG | At4g18960 | 10.4 ± 2.0 | 19.2 ± 2.4 | 0.54 | 0.0033 | 0.0899 |
| ELF4 | At2g40080 | 44.0 ± 4.6 | 79.8 ± 18.1 | 0.55 | 0.0106 | 0.1474 |
| PI | At5g20240 | 8.9 ± 1.9 | 21.5 ± 5.2 | 0.41 | 0.0050 | 0.1077 |
| NAC2 | At5g61430 | 24.1 ± 11.0 | 124.0 ± 18.5 | 0.19 | 0.0011 | 0.0575 |
| NAP | At1g69490 | 76.9 ± 20.3 | 330.7 ± 11.0 | 0.23 | 0.0001 | 0.0241 |

### Example 15

### ZmARGOS1 is functional very early in the ethylene signaling pathway

To determine where ZmARGOS1 acts in the genetically established ethylene signaling pathway, genetic analysis was performed by introducing the 35S:ZmARGOS1 construct into homozygous ctrl-1 mutant. Thirty events were analyzed for ethylene response. The light-grown transgenic plants overexpressing ZmARGOS1 displayed the characteristic constitutive ethylene response phenotype, as the ctrl-1 mutant did (FIG. 16A). The etiolated seedling exhibited the triple response in the absence of ACC (FIG. 16B), demonstrating that CTR1 is epistatic to ZmARGOS1. Because CTR1 directly interacts with ethylene receptors in the ethylene signaling pathway, the genetic analysis revealed that ZmARGOS1 functions very early in the ethylene signaling pathway.

### Example 16

### Overexpression of AtARGOS2, AtARGOS3 and AtARGOS4 Decreases Ethylene Sensitivity in Arabidopsis

To determine if other maize and Arabidopsis TPT domain-containing proteins can modulate ethylene response, the maize ARGOS7, ARGOS8 and ARGOS9 and Arabidopsis AtARGOS2, AtARGOS3 and AtARGOS4 genes were overexpressed in Arabidopsis under the control of the CaMV 35S promoter. For each construct, twenty-five transgenic T1 seeds, each likely an independent event were randomly selected based on expression of the YFP marker gene and plated on 1/2 MS medium with or without ACC. The 35S:ZmARGOS9 and 35S:ZmARGOS7 plants displayed the ethylene insensitive phenotype in 3-day-old seedlings in the presence of 10 μM ACC, as the 35S:ZmARGOS1 plants did (FIG. 17A). The adult plants exhibited the phenotype of enlarged leaves. Floral transition was delayed by 3 to 8 days and abscission of the perianth organs was also delayed. Overexpression of ZmARGOS8 significantly reduced the ethylene response in etiolated seedlings, but the phenotype was weaker than that of ZmARGOS1 (FIG. 17A).

Etiolated seedlings of transgenic Arabidopsis overexpressing Arabidopsis AtARGOS3 and AtARGOS4 were insensitive to 10 μM ACC (FIG. 17A). The adult plants showed similar phenotypes to the 35S:ZmARGOS1 transgenics. The effect of Arabidopsis AtARGOS2 on ethylene sensitivity was weak relative to AtARGOS3, AtARGOS4 and maize ZmARGOS1. In the presence of 10 μM ACC, the morphology of the etiolated 35S:AtARGOS2 seedlings were similar to the wild-type Col-0 (data not shown), but hypocotyls and roots were significantly longer than those in wild-type control plants at 1.0 and 2.5 μM ACC (FIG. 17B). The flowering of the light-grown 35S:AtARGOS2 plants was delayed by 0.5 to 2.5 days in average in comparison to wild-type plants.

### Example 17

### The TPT Domain is Sufficient to Confer Ethylene Insensitivity in Arabidopsis

Because the maize ARGOS genes all contain the TM1-PRM-TM2 domain, it was hypothesized that the TPT domain may be responsible for the common function of the genes in modulating ethylene responses. Truncation and mutation experiments were conducted with

ARGOS1 to test the hypothesis. Deletion of the N-terminal region (aa2-61) had no effect on ARGOS1 function of conferring ethylene insensitivity in Arabidopsis (FIG. 18). Neither did the C-terminal sequence deletion (aa135-144). Transgenic plants expressing a truncated ZmARGOS1 with 61 amino acid residues removed from the N-terminus and 10 from the C-terminus displayed the same ethylene insensitive phenotype as the full-length ZmARGOS1 in etiolated seedlings and light-grown adult plants. The functional, truncated ZmARGOS1 contains only the two transmembrane helices and the 8-amino acid proline-rich loop.

Mutation of two amino acids in the first transmembrane domain (SEQ ID NO: 90) (P83D and A84D) which would disrupt the helix structure abolished the capability of ZmARGOS1 in conferring ethylene insensitivity (FIG. 18). The same result was obtained when the second transmembrane domain (SEQ ID NO: 91) was disrupted by substituting three amino acids (L120D, L121D and L122D) in the helix region. These results showed that the transmembrane domains are required for the function of ethylene sensitivity modification. To assess the role of PRM (SEQ ID NO: 88), each of the eight amino acids was substituted with aspartate and the variants were overexpressed in Arabidopsis. The etiolated seedling assay with 10 μM ACC revealed that amino acids L104, P106 and P107 are crucial for conferring ethylene insensitivity (FIG. 19). The mutation of P102D, P103D and P108D allows root and hypocotyl elongation in etiolated seedlings in the presence of ACC, but the root and hypocotyl were much shorter than that of the wild-type ZmARGOS1, indicating that these three prolines are also important for ARGOS1 function. The mutation of P105D and S109D (SEQ ID NO: 102, variables indicated as SEQ ID NO: 96) had no effect on ARGOS1 in terms of modulating ethylene sensitivity in Arabidopsis.

### Example 18

### Maize ARGOS1 is Localized in the ER Membranes

Sequence analysis predicated that maize ARGOS1 and other family members are membrane proteins, but in Arabidopsis ARGOS1 was reported to present in the nucleus, cytoplasm and cytoplasmic membranes. To clarify this difference, maize ARGOS1 was tagged with the FLAG-HA epitope at either the N- or C-terminus and overexpressed in Arabidopsis under the control of the CaMV 35S promoter. The transgenic plants expressing either the N-tagged or C-tagged ZmARGOS1 displayed the ethylene insensitive phenotype indistinguishable from that in untagged ZmARGOS1. Cell fractionation was performed to separate the soluble and microsomal fraction. The tagged ZmARGOS1 protein was detected in the membrane fraction, but not in the soluble fraction with Western blotting analysis using the anti-FLAG antibody (FIG. 20A), reaffirming that maize ARGOS1 is a membrane protein.

The subcellular localization of ZmARGOS1 was determined by using the green fluorescent protein (GFP) tagging technology. Fusing AcGFP to the C-terminus of ZmARGOS1 did not affect ZmARGOS1 function in conferring ethylene insensitivity. However, the N-terminal fusion protein was inactive. Transgenic plants overexpressing the C-terminal fusion protein were examined under an epi-fluorescence microscope. Green fluorescence was associated with a network that morphologically resembles the ER in hypocotyl cells of stable transgenic Arabidopsis plants and onion epidermal cells transiently expressing ZmARGOS1-AcGFP fusion protein (FIG. 20B). The fusion protein co-localized with the ER marker (ER-ck CD3-953) in the onion epidermal cells (FIG. 20C). Green fluorescence was also observed in a granular form (FIGS. 20B and 20D), which was co-localized with the Golgi marker (G-ck CD3-961). Nuclei were free from green fluorescence and no evidence was obtained for the presence of the fusion protein in the plasmamembrane or tonoplast membrane.

### Example 19

### Plant Materials and Growth Conditions

The Arabidopsis thaliana mutant etol-1 and ctrl-1 are in the Columbia (Col-0) ecotype and were obtained from Arabidopsis Biological Resource Center (Columbus, OH). Plants were grown under fluorescent lamps supplemented with incandescent lights (approximate 120 mE m⁻² s⁻¹) in growth chambers with 16 h light period at 24° C. and 8 hr dark period at 23° C. and 50% relative humidity. Seeds were sown in soil and stratified at 4° C. for 4 days before moving into the growth chamber. Plants were fertilized once at flowering time with mineral nutrients. For seedling analysis, seeds were surface-sterilized, stratified and plated on medium containing Murashige and Skoog inorganic salts at half concentration, 1% sucrose and 0.8% agar.

For the triple-response assay, surface sterilized seeds were germinated and seedlings grown in the presence of ethylene gas (Praxair, Danbury, Conn.) in an airtight container or on medium containing ACC (Calbiochem, La Jolla, Calif.) at the stated concentrations. Hypocotyls and roots were measured by photographing the seedlings with a digital camera and using image analysis software.

For assaying the maize seedling response to ACC, seeds were germinated with the filter paper method. Filter papers were wetted in an ACC aqueous solution at stated concentrations and the rolled-up seeds were placed in the same solution at 24° C. in the dark. Seedling phenotypes were scored in 5 days. For gene expression analysis, maize V3 plants grown in greenhouse were sprayed with various hormones and leaf tissues were used for RNA extraction.

### Ethylene Measurements

Whole leaves were excised from 3-week-old Arabidopsis and leaf discs were punctured from two uppermost collared leaves of V7 maize plants. After letting the wound-induced ethylene burst subside for two hours, the leaves or leaf discs then were placed in 9.77-ml amber vials containing a filter paper disc wetted with 50 μl of distilled water and sealed with aluminum crimp seals. After a 20-h incubation period, 1-ml samples were taken from the headspace of each sealed vial. The ethylene content was quantified by gas chromatography. Ethylene production rate was expressed as nL per hour per gram of fresh weight.

### Gene Expression Analysis by RNA-Seq

Total RNAs were isolated from aerial tissues of 19-day-old Arabidopsis plants by use of the Qiagen RNeasy kit for total RNA isolation (Qiagen, Germantown, Md.). Sequencing libraries from the resulting total RNAs were prepared using the TruSeq mRNA-Seq kit according to the manufacturer's instructions (Illumina, San Diego, Calif.). Briefly, mRNAs were isolated via attachment to oligo(dT) beads, fragmented to a mean size of 150nt, reverse transcribed into cDNA using random primers, end repaired to create blunt end fragments, 3′ A-tailed, and ligated with Illumina indexed TruSeq adapters. Ligated cDNA fragments were PCR amplified using Illumina TruSeq primers and purified PCR products were checked for quality and quantity on the Agilent Bioanalyzer DNA 7500 chip (Agilent Technologies, Santa Clara, Calif.). Ten nanomolar pools made up of three samples with unique indices were generated. Pools were sequenced using TruSeq Illumina GAIIx indexed sequencing. Each pool of three was hybridized to a single flowcell lane and was amplified, blocked, linearized and primer hybridized using the Illumina cBot. Sequencing was completed on the Genome Analyzer IIx. Fifty base pairs of insert sequence and six base pairs of index sequence were generated. Sequences were trimmed based on quality scores and de-convoluted based on index identifier. Resulting sequences were bowtie aligned to Arabidopsis gene set and normalized to Relative Parts Per Kilobase Per Ten Million (RPKtM). The generated RPKtM data matrix was visualized and analyzed in GeneData Analyst software (Genedata AG, Basel, Switzerland).

### Nucleic Acid Analysis

Total RNA was extracted from Arabidopsis or maize leaf tissues, separated by electrophoresis in a 1% (w/v) agarose/formaldehyde/MOPS gel and blotted to a nylone membrane. Probe labeling, hybridization and washing were carried out according to the manufacturer's instructions.

### Membrane Fractionation

Microsomal membranes and soluble fraction were isolated from 3-week-old Arabidopsis plants grown in a growth chamber using homogenization buffer containing 30 mM Tris (pH 7.6), 150 mM NaCl, 0.1 mM EDTA, 20% (v/v) glycerol and protease inhibitors (Sigma-Aldrich, St. Louis, Mo.). The homogenate was filtered through two layers of Miracloth and centrifuged for 10 min at 5,000 g to remove cell debris and cell walls. The supernatant was then centrifuged at 100,000 g for 90 min, and the resulting membrane pellet resuspended in 10 mM Tris (pH 7.6), 150 mM NaCl, 0.1 mM EDTA, 10% (v/v) glycerol and protease inhibitors.

### Immunoblotting

Protein was separated by SDS-PAGE, blotted to a PVDF membrane and probed with monoclonal anti-FLAG (Sigma-Aldrich, St. Louis, Mo.) or polyclonal anti-BiP (Santa Cruz Biotechnology, Santa Cruz, Calif.) antibodies according to the manufacturer's instructions. The primary antibodies were detected with the Pierce Fast Western Blot Kit, ECL Substrate (Thermo Scientific, Rockford, Ill.).

### Fluorescence Microscopy

Seedlings were harvested and immediately placed in PBS (pH7.2) on glass slides for microscopic observations. Observations and images were taken with a Leica (Wetzlar, Germany) DMRXA epi-fluorescence microscope with a mercury light source. Two different fluorescent filter sets were used to monitor AcGFP fluorescence, Alexa 488 #MF-105 (exc. 486-500, dichroic 505LP, em. 510-530) and Red-Shifted GFP #41001 (exc. 460-500, dichroic 505LP, em. 510-560) both from Chroma Technology (Bellows Falls, VT). Images were captured with a Photometrics (Tucson, Ariz.) CooISNAP HQ CCD. Camera and microscope were controlled, and images manipulated by Molecular Devices (Downingtown, Pa.) MetaMorph imaging software.

### Example 20

### Analysis of Conserved Regions of Various Species

Two alignments were prepared, showing proline rich domains and transmembrane domains across various species.

FIG. 12 shows the sequence alignment of the ARGOS genes to show the conserved region among the family members and homologs across grass species. Conserved region is identified as LX1X2LPLX3LPPLX4X5PP (SEQ ID NO: 86) where X1=L,V,I; X2=L,V,I,F; X3=V,L,A; X4=P,Q,S; X5=P,A.

FIG. 21 shows the alignment of ARGOS polypeptide sequences from various species identifying conserved transmembrane segments. Information is labeled as follows:

ID=SEQ ID, although grass sp. are identified per Table 1 as ARGOS #

St=sequence start number in the aligned sequence panel,

Ed=sequence ending number in the aligned sequence panel,

TMH1/2=transmembrane segments,

Ident/TMH1,2=ratio of identity.

Alignment produced by Clustalw with ZmARGOS8 (SEQ ID NO: 44) as the aligning profile. The identity calculation is as compared to ZmARGOS8.

### Example 21

### Vectors for ARGOS8

A series of vectors were prepared for ZmARGOS8 transformation into plant tissue. Promoters selected included but were not limited to: UBI, ROOTMET2, BSV(AY)TR, OsACTIN, ZmPEPC1, ZmCYCLO1, AtHSP, for example, in addition to other tissue and temporally expressed promoters. Drought inducible promoters such as Rab17 were also used.

### Example 22

### Soybean Embryo Transformation

Soybean embryos are bombarded with a plasmid containing an ARGOS sequence operably linked to an ubiquitin promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein, et al., (1987) Nature (London) 327:70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell, et al., (1985) Nature 313:810-812), the hygromycin phosphotransferase gene from plasmid pJR225 (from E. coil; Gritz, et al., (1983) Gene 25:179-188) and the 3′ region of the nopaline synthase gene from the T-DNA of the Ti plasmid of Agrobacterium tumefaciens. The expression cassette comprising an ARGOS sense sequence operably linked to the ubiquitin promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 23

### Sunflower Meristem Tissue Transformation

Sunflower meristem tissues are transformed with an expression cassette containing an ARGOS sequence operably linked to a ubiquitin promoter as follows (see also, EP Patent Number EP 0 486233, herein incorporated by reference and Malone-Schoneberg, et al., (1994) Plant Science 103:199-207). Mature sunflower seed (Helianthus annuus L.) are dehulled using a single wheat-head thresher. Seeds are surface sterilized for 30 minutes in a 20% Clorox® bleach solution with the addition of two drops of Tween® 20 per 50 ml of solution. The seeds are rinsed twice with sterile distilled water.

Split embryonic axis explants are prepared by a modification of procedures described by Schrammeijer, et al., (Schrammeijer, et al., (1990) Plant Cell Rep. 9:55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige, et al., (1962) Physiol. Plant., 15:473-497), Shepard's vitamin additions (Shepard, (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA₃), pH 5.6 and 8 g/l Phytagar.

The explants are subjected to microprojectile bombardment prior to Agrobacterium treatment (Bidney, et al., (1992) Plant Mol. Biol. 18:301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA105 is used in all transformation experiments. A binary plasmid vector comprising the expression cassette that contains the ARGOS gene operably linked to the ubiquitin promoter is introduced into Agrobacterium strain EHA105 via freeze-thawing as described by Holsters, et al., (1978) Mol. Gen. Genet. 163:181-187. This plasmid further comprises a kanamycin selectable marker gene (i.e, nptII). Bacteria for plant transformation experiments are grown overnight (28° C. and 100 RPM continuous agitation) in liquid YEP medium (10 gm/l yeast extract, 10 gm/l Bacto®peptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for a modulation in meristem development (i.e., an alteration of size and appearance of shoot and floral meristems).

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite®, pH 5.6) and grown under conditions described for explant culture. The upper portion of the seedling is removed, a 1 cm vertical slice is made in the hypocotyl, and the transformed shoot inserted into the cut. The entire area is wrapped with parafilm® to secure the shoot. Grafted plants can be transferred to soil following one week of in vitro culture. Grafts in soil are maintained under high humidity conditions followed by a slow acclimatization to the greenhouse environment. Transformed sectors of T₀ plants (parental generation) maturing in the greenhouse are identified by NPTII ELISA and/or by ARGOS activity analysis of leaf extracts while transgenic seeds harvested from NPTII-positive T₀ plants are identified by ARGOS activity analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox® bleach solution with the addition of two to three drops of Tween® 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA, and 0.8% Phytagar at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar), and then cultured on the medium for 24 hours in the dark. Approximately 18.8 mg of 1.8 μm tungsten particles are resuspended in 150 μl absolute ethanol. After sonication, 8 μl of it is dropped on the center of the surface of macrocarrier. Each plate is bombarded twice with 650 psi rupture discs in the first shelf at 26 mm of Hg helium gun vacuum.

The plasmid of interest is introduced into Agrobacterium tumefaciens strain EHA105 via freeze thawing as described previously. The pellet of overnight-grown bacteria at 28° C. in a liquid YEP medium (10 g/l yeast extract, 10 g/l Bacto®peptone and 5 g/l NaCl, pH 7.0) in the presence of 50 μg/l kanamycin is resuspended in an inoculation medium (12.5 mM 2-mM 2-(N-morpholino) ethanesulfonic acid, MES, 1 g/l NH₄Cl and 0.3 g/l MgSO₄ at pH 5.7) to reach a final concentration of 4.0 at OD₆₀₀. Particle-bombarded explants are transferred to GBA medium (374E), and a droplet of bacteria suspension is placed directly onto the top of the meristem.

The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour day and 26° C. incubation conditions.

Explants (around 2 cm long) from two weeks of culture in 374C medium are screened for a modulation in meristem development (i.e., an alteration of size and appearance of shoot and floral meristems). After positive (i.e., a change in ARGOS expression) explants are identified, those shoots that fail to exhibit an alteration in ARGOS activity are discarded, and every positive explant is subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate protein activity assay. At this time, the positive shoots recovered from a single node will generally have been enriched in the transgenic sector detected in the initial assay prior to nodal culture.

Recovered shoots positive for altered ARGOS expression are grafted to Pioneer® hybrid 6440 in vitro-grown sunflower seedling rootstock. The rootstocks are prepared in the following manner. Seeds are dehulled and surface-sterilized for 20 minutes in a 20% Clorox® bleach solution with the addition of two to three drops of Tween® 20 per 100 ml of solution, and are rinsed three times with distilled water. The sterilized seeds are germinated on the filter moistened with water for three days, then they are transferred into 48 medium (half-strength MS salt, 0.5% sucrose, 0.3% gelrite® pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with parafilm®. After one week of culture on the medium, grafted plants are transferred to soil. In the first two weeks, they are maintained under high humidity conditions to acclimatize to a greenhouse environment.

### Example 24

### Rice Callus Transformation

One method for transforming DNA into cells of higher plants that is available to those skilled in the art is high-velocity ballistic bombardment using metal particles coated with the nucleic acid constructs of interest (see, Klein, et al., (1987) Nature (London) 327:70-73 and see, U.S. Pat. No. 4,945,050). A Biolistic PDS-1000/He (BioRAD Laboratories, Hercules, Calif.) is used for these complementation experiments. The particle bombardment technique is used to transform the ZM-CIPK1 mutants and wild type rice with two genomic DNA fragments:
1) 10.0 kb MunI fragment from wild type that includes the 4.5 kb upstream and 3.8 kb downstream region of the ZM-CIPK1 gene,
2) 5.1 kb EcoRI fragment from wild type that includes the 1.7 kb upstream and 1.7 kb downstream region of the ZM-CIPK1 gene.

The bacterial hygromycin B phosphotransferase (Hpt II) gene from Streptomyces hygroscopicus that confers resistance to the antibiotic is used as the selectable marker for rice transformation. In the vector, pML18, the Hpt II gene was engineered with the 35S promoter from Cauliflower Mosaic Virus and the termination and polyadenylation signals from the octopine synthase gene of Agrobacterium tumefaciens. pML18 was described in WO 1997/47731, which was published on Dec. 18, 1997, the disclosure of which is hereby incorporated by reference.

Embryogenic callus cultures derived from the scutellum of germinating rice seeds serve as source material for transformation experiments. This material is generated by germinating sterile rice seeds on a callus initiation media (MS salts, Nitsch and Nitsch vitamins, 1.0 mg/l 2,4-D and 10 μM AgNO₃) in the dark at 27-28° C. Embryogenic callus proliferating from the scutellum of the embryos is the transferred to CM media (N6 salts, Nitsch and Nitsch vitamins, 1 mg/l 2,4-D, Chu, et al., (1985) Sci. Sinica 18:659-668). Callus cultures are maintained on CM by routine sub-culture at two week intervals and used for transformation within 10 weeks of initiation.

Callus is prepared for transformation by subculturing 0.5-1.0 mm pieces approximately 1 mm apart, arranged in a circular area of about 4 cm in diameter, in the center of a circle of Whatman® #541 paper placed on CM media. The plates with callus are incubated in the dark at 27-28° C. for 3-5 days. Prior to bombardment, the filters with callus are transferred to CM supplemented with 0.25 M mannitol and 0.25 M sorbitol for 3 hr in the dark. The petri dish lids are then left ajar for 20-45 minutes in a sterile hood to allow moisture on tissue to dissipate.

Each genomic DNA fragment is co-precipitated with pML18 containing the selectable marker for rice transformation onto the surface of gold particles. To accomplish this, a total of 10 μg of DNA at a 2:1 ratio of trait:selectable marker DNAs are added to 50 μl aliquot of gold particles that have been resuspended at a concentration of 60 mg ml⁻¹. Calcium chloride (50 μl of a 2.5 M solution) and spermidine (20 μl of a 0.1 M solution) are then added to the gold-DNA suspension as the tube is vortexing for 3 min. The gold particles are centrifuged in a microfuge for 1 sec and the supernatant removed. The gold particles are then washed twice with 1 ml of absolute ethanol and then resuspended in 50 μl of absolute ethanol and sonicated (bath sonicator) for one second to disperse the gold particles. The gold suspension is incubated at −70° C. for five minutes and sonicated (bath sonicator) if needed to disperse the particles. Six μl of the DNA-coated gold particles are then loaded onto mylar macrocarrier disks and the ethanol is allowed to evaporate.

At the end of the drying period, a petri dish containing the tissue is placed in the chamber of the PDS-1000/He. The air in the chamber is then evacuated to a vacuum of 28-29 inches Hg. The macrocarrier is accelerated with a helium shock wave using a rupture membrane that bursts when the He pressure in the shock tube reaches 1080-1100 psi. The tissue is placed approximately 8 cm from the stopping screen and the callus is bombarded two times. Two to four plates of tissue are bombarded in this way with the DNA-coated gold particles. Following bombardment, the callus tissue is transferred to CM media without supplemental sorbitol or mannitol.

Within 3-5 days after bombardment the callus tissue is transferred to SM media (CM medium containing 50 mg/I hygromycin). To accomplish this, callus tissue is transferred from plates to sterile 50 ml conical tubes and weighed. Molten top-agar at 40° C. is added using 2.5 ml of top agar/100 mg of callus. Callus clumps are broken into fragments of less than 2 mm diameter by repeated dispensing through a 10 ml pipet. Three ml aliquots of the callus suspension are plated onto fresh SM media and the plates are incubated in the dark for 4 weeks at 27-28° C. After 4 weeks, transgenic callus events are identified, transferred to fresh SM plates and grown for an additional 2 weeks in the dark at 27-28° C.

Growing callus is transferred to RM1 media (MS salts, Nitsch and Nitsch vitamins, 2% sucrose, 3% sorbitol, 0.4% gelrite® +50 ppm hyg B) for 2 weeks in the dark at 25° C. After 2 weeks the callus is transferred to RM2 media (MS salts, Nitsch and Nitsch vitamins, 3% sucrose, 0.4% gelrite® +50 ppm hyg B) and placed under cool white light (˜40 μEm⁻¹s⁻¹) with a 12 hr photoperiod at 25° C. and 30-40% humidity. After 2-4 weeks in the light, callus begin to organize, and form shoots. Shoots are removed from surrounding callus/media and gently transferred to RM3 media (½×MS salts, Nitsch and Nitsch vitamins, 1% sucrose+50 ppm hygromycin B) in phytatrays™ (Sigma Chemical Co., St. Louis, Mo.) and incubation is continue using the same conditions as described in the previous step.

Plants are transferred from RM3 to 4″ pots containing Metro mix 350 after 2-3 weeks, when sufficient root and shoot growth have occurred. The seed obtained from the transgenic plants is examined for genetic complementation of the construct with the wild-type genomic DNA containing ARGOS8 gene.

### Example 25

### Aqrobacterium Mediated Grass Transformation

Grass plants may be transformed by following the Agrobacterium mediated transformation of Luo, et al., (2004) Plant Cell Rep 22:645-652.

### Materials and Methods

### Plant Material

A commercial cultivar of creeping bentgrass (Agrostis stolonifera L., cv. Penn-A-4) supplied by Turf-Seed (Hubbard, Oreg.) can be used. Seeds are stored at 4° C. until used.

### Bacterial Strains and Plasmids

Agrobacterium strains containing one of 3 vectors are used. One vector includes a pUbi-gus/Act1-hyg construct consisting of the maize ubiquitin (ubi) promoter driving an intron-containing b-glucuronidase (GUS) reporter gene and the rice actin 1 promoter driving a hygromycin (hyg) resistance gene. The other two pTAP-arts/35S-bar and pTAP-barnase/Ubi-bar constructs are vectors containing a rice tapetum-specific promoter driving either a rice tapetum-specific antisense gene, rts (Lee, et al., (1996) Int Rice Res Newsl 21:2-3) or a ribonuclease gene, barnase (Hartley, (1988) J Mol Biol 202:913-915), linked to the cauliflower mosaic virus 35S promoter (CaMV 35S) or the rice ubi promoter (Huq, et aL, (1997) Plant Physiol 113:305) driving the bar gene for herbicide resistance as the selectable marker.

### Induction of Embryogenic Callus and Agrobacterium-Mediated Transformation

Mature seeds are dehusked with sand paper and surface sterilized in 10% (v/v) Clorox® bleach (6% sodium hypochlorite) plus 0.2% (v/ v) Tween® 20 (Polysorbate 20) with vigorous shaking for 90 min. Following rinsing five times in sterile distilled water, the seeds are placed onto callus-induction medium containing MS basal salts and vitamins (Murashige and Skoog, (1962) Physiol Plant 15:473-497), 30 g/l sucrose, 500 mg/l casein hydrolysate, 6.6 mg/l 3,6-dichloro-o-anisic acid (dicamba), 0.5 mg/l 6-benzylaminopurine (BAP) and 2 g/l Phytagel. The pH of the medium is adjusted to 5.7 before autoclaving at 120° C. for 20 min. The culture plates containing prepared seed explants are kept in the dark at room temperature for 6 weeks. Embryogenic calli are visually selected and subcultured on fresh callus-induction medium in the dark at room temperature for 1 week before co-cultivation.

### Transformation

The transformation process is divided into five sequential steps: agro-infection, co-cultivation, antibiotic treatment, selection and plant regeneration. One day prior to agro-infection, the embryogenic callus is divided into 1- to 2-mm pieces and placed on callus-induction medium containing 100 μM acetosyringone. A 10-ml aliquot of Agrobacterium suspension (OD=1.0 at 660 nm) is then applied to each piece of callus, followed by 3 days of co-cultivation in the dark at 25″C. For the antibiotic treatment step, the callus is then transferred and cultured for 2 weeks on callus-induction medium plus 125 mg/l cefotaxime and 250 mg/l carbenicillin to suppress bacterial growth. Subsequently, for selection, the callus is moved to callus-induction medium containing 250 mg/l cefotaxime and 10 mg/l phosphinothricin (PPT) or 200 mg/l hygromycin for 8 weeks. Antibiotic treatment and the entire selection process is performed at room temperature in the dark. The subculture interval during selection is typically 3 weeks. For plant regeneration, the PPT- or hygromycin-resistant proliferating callus is first moved to regeneration medium (MS basal medium, 30 g/l sucrose, 100 mg/l myo-inositol, 1 mg/l BAP and 2 g/l Phytagel) supplemented with cefotaxime, PPT or hygromycin. These calli are kept in the dark at room temperature for 1 week and then moved into the light for 2-3 weeks to develop shoots. Small shoots are then separated and transferred to hormone-free regeneration medium containing PPT or hygromycin and cefotaxime to promote root growth while maintaining selection pressure and suppressing any remaining Agrobacterium cells. Plantlets with well-developed roots (3-5 weeks) are then transferred to soil and grown either in the greenhouse or in the field.

### Staining for GUS Activity

GUS activity in transformed callus is assayed by histochemical staining with 1 mM 5-bromo-4-chloro-3-indolyl-b-d-glucuronic acid (X-Gluc, Biosynth, Staad, Switzerland) as described in Jefferson, (1987) Plant Mol Biol Rep 5:387-405. The hygromycin-resistant callus surviving from selection was incubated at 37° C. overnight in 100 μl of reaction buffer containing X-Gluc. GUS expression is then documented by photography.

### Vernalization and Out-Crossing of Transgenic Plants

Transgenic plants are maintained out of doors in a containment nursery (3-6 months) until the winter solstice in December. The vernalized plants are then transferred to the greenhouse and kept at 25° C. under a 16/8 h [day/light (artificial light)] photoperiod and surrounded by non-transgenic wild-type plants that physically isolated them from other pollen sources. The plants will initiate flowering 3-4 weeks after being moved back into the greenhouse. They are out-crossed with the pollen from the surrounding wild-type plants. The seeds collected from each individual transgenic plant are germinated in soil at 25° C. and T1 plants are grown in the greenhouse for further analysis.

### Seed Testing

### Test of the Transgenic Plants and Their Progeny for Resistance to PPT

Transgenic plants and their progeny are evaluated for tolerance to glufosinate (PPT) indicating functional expression of the bar gene. The seedlings are sprayed twice at concentrations of 1-10% (v/v) Finale© (AgrEvo USA, Montvale, N.J.) containing 11% glufosinate as the active ingredient. Resistant and sensitive seedlings are clearly distinguishable 1 week after the application of Finale in all the sprayings.

### Statistical Analysis

Transformation efficiency for a given experiment is estimated by the number of PPT-resistant events recovered per 100 embryogenic calli infected and regeneration efficiency is determined using the number of regenerated events per 100 events attempted. The mean transformation and regeneration efficiencies are determined based on the data obtained from multiple independent experiments. A Chi-square test can be used to determine whether the segregation ratios observed among T1 progeny for the inheritance of the bar gene as a single locus fit the expected 1:1 ratio when out-crossed with pollen from untransformed wild-type plants.

### DNA Extraction and Analysis

Genomic DNA is extracted from approximately 0.5-2 g of fresh leaves essentially as described by Luo, et al., (1995) Mol Breed 1:51-63. Ten micrograms of DNA is digested with HindIII or BamHI according to the supplier's instructions (New England Biolabs, Beverly, Mass.). Fragments are size-separated through a 1.0% (w/v) agarose gel and blotted onto a Hybond-N+ membrane (Amersham Biosciences, Piscataway, N.J.). The bar gene, isolated by restriction digestion from pTAP-arts/35S-bar, is used as a probe for Southern blot analysis. The DNA fragment is radiolabeled using a Random Priming Labeling kit (Amersham Biosciences) and the Southern blots are processed as described by Sambrook, et al., (1989) Molecular cloning: a laboratory manual, 2nd edn. Cold Spring Harbor Laboratory Press, New York.

### Polytnerase Chain Reaction

The two primers designed to amplify the bar gene are as follows: 5′-GTCTGCACCATCGTCAACC-3′ (SEQ ID NO: 94), corresponding to the proximity of the 5′ end of the bar gene and 5′-GAAGTCCAGCTGCCAGAAACC-3′ (SEQ ID NO: 95), corresponding to the 3′ end of the bar coding region. The amplification of the bar gene using this pair of primers should result in a product of 0.44 kb. The reaction mixtures (25 μl total volume) consist of 50 mM KCl, 10 mM Tris-HCl (pH 8.8), 1.5 mM MgCl2, 0.1% (w/v) Triton X-100, 200 μM each of dATP, dCTP, dGTP and dTTP, 0.5 μM of each primer, 0.2 μg of template DNA and 1 U Taq DNA polymerase (QIAGEN, Valencia, Calif.). Amplification is performed in a Stratagene Robocycler Gradient 96 thermal cycler (La Jolla, Calif.) programmed for 25 cycles of 1 min at 94° C. (denaturation), 2 min at 55° C. (hybridization), 3 min at 72° C. (elongation) and a final elongation step at 72° C. for 10 min. PCR products are separated on a 1.5% (w/v) agarose gel and detected by staining with ethidium bromide.

### Example 26

### Sugar Cane Transformation

This protocol describes routine conditions for production of transgenic sugarcane lines. The same conditions are close to optimal for number of transiently expressing cells following bombardment into embryogenic sugarcane callus. See also, Bower, et al., (1996). Molec Breed 2:239-249; Birch and Bower, (1994). Principles of gene transfer using particle bombardment. In Particle Bombardment Technology for Gene Transfer, Yang and Christou, eds (New York: Oxford University Press), pp. 3-37 and Santosa, et al., (2004), Molecular Biotechnology 28:113-119, incorporated herein by reference.

### Sugarcane Transformation Protocol

1. Subculture callus on MSC3, 4 days prior to bombardment:
   (a) Use actively growing embryogenic callus (predominantly globular pro-embryoids rather than more advanced stages of differentiation) for bombardment and through the subsequent selection period.
   (b) Divide callus into pieces around 5 mm in diameter at the time of subculture and use forceps to make a small crater in the agar surface for each transferred callus piece.
   (c) Incubate at 28° C. in the dark, in deep (25 mm) Petri dishes with micropore tape seals for gas exchange.
2. Place embryogenic callus pieces in a circle (˜2.5 cm diameter), on MSC3Osm medium. Incubate for 4 hours prior to bombardment.
3. Sterilize 0.7 pm diameter tungsten (Grade M-10, Bio-Rad #165-2266) in absolute ethanol. Vortex the suspension, then pellet the tungsten in a microfuge for ˜30 seconds. Draw off the supernatant and resuspend the particles at the same concentration in sterile H₂O. Repeat the washing step with sterile H₂0 twice and thoroughly resuspend particles before transferring 50 μl aliquots into microfuge tubes.
4. Add the precipitation mix components:

| Component (stock solution) | Volume to add | Final conc in mix |
|---|---|---|
| Tungsten (100 μg/μl in H₂0) | 50 μl | 38.5 μg/μl |
| DNA (1 μg/μl) | 10 μl | 0.38 μg/μl |
| CaCl₂ (2.5M in H20) | 50 μl | 963 mM |
| Spermidine free base (0.1M in H₂0) | 20 μl | 15 mM |

5. Allow the mixture to stand on ice for 5 min. During this time, complete steps 6-8 below.
6. Disinfect the inside of the ‘gene gun’ target chamber by swabbing with ethanol and allow it to dry.
7. Adjust the outlet pressure at the helium cylinder to the desired bombardment pressure.
8. Adjust the solenoid timer to 0.05 seconds. Pass enough helium to remove air from the supply line (2-3 pulses).
9. After 5 min on ice, remove (and discard) 100 μl of supernatant from the settled precipitation mix.
10. Thoroughly disperse the particles in the remaining solution.
11. Immediately place 4 μl of the dispersed tungsten-DNA preparation in the center of the support screen in a 13 mm plastic syringe filter holder.
12. Attach the filter holder to the helium outlet in the target chamber.
13. Replace the lid over the target tissue with a sterile protective screen. Place the sample into the target chamber, centered 16.5 cm under the particle source and close the door.
14. Open the valve to the vacuum source. When chamber vacuum reaches 28″ of mercury, press the button to apply the accelerating gas pulse, which discharges the particles into the target chamber.
15. Close the valve to the vacuum source. Allow air to return slowly into the target chamber through a sterilizing filter. Open the door, cover the sample with a sterile lid and remove the sample dish from the chamber.
16. Repeat steps 10-15 for consecutive target plates using the same precipitation mix, filter and screen.
17. Approximately 4 hours after bombardment, transfer the callus pieces from MSC3Osm to MSC3.
18. Two days after shooting, transfer the callus onto selection medium. During this transfer, divide the callus into pieces ˜5mm in diameter, with each piece being kept separate throughout the selection process.
19. Subculture callus pieces at 2-3 week intervals.
20. When callus pieces grow to ˜5 to 10 mm in diameter (typically 8 to 12 weeks after bombardment) transfer onto regeneration medium at 28° C. in the light.
21. When regenerated shoots are 30-60 mm high with several well-developed roots, transfer them into potting mix with the usual precautions against mechanical damage, pathogen attack and desiccation until plantlets are established in the greenhouse.

### Example 27

### ZmARGOS8 Analysis in Arabidopsis thaliana Seedling

Five ZmARGOS8 events and one ZmARGOS1 event were analyzed in 3 day old, etiolated Arabidopsis seedlings. Measurements of hypocotyls length and root length were performed in seedlings exposed to 10 uM ACC. Results indicated that there was reduced ethylene sensitivity in ZmARGOS8 transgenic Arabidopsis seedlings, and that the phenotype for the ZmARGOS8 plants was weaker than the ZmARGOS1 plants. Hypocotyl length of control plants was approximately 2 mm, while ZmARGOS8 plants ranged from 2.8-4 mm and ZmARGOS1 seedlings averaged nearly 5mm. Root length measurements included control plants at 1 mm, ZmARGOS8 seedlings ranging from 1.5-4.25 mm and ZmARGOS1 seedings averaging 5.5 mm.

### Example 28

### TPT Domain is Responsible for the Ethylene Insensitive Phenotype

3-day old Arabidopsis seedlings, transformed with either the ZmARGOS8 or a truncated ZmARGOS8 (TR), along with an empty vector control, were exposed to 10 uM ACC during growth. Measurements of the seedling development across the 3 groups indicated while both ARGOS8 and the ARGOS8TR both had increased ethylene insensitivity and increased tissue growth, the truncated version of ARGOS8 caused a stronger phenotypic response than the full-length ZmARGOS8 seedlings.

### Example 29

### Transgenic Hybrid Plants Overexpressing ZmARGOS1 Improved Traits Related to Stress Tolerance

Transgenic hybrid plants overexpression ZmARGOS1, grown in the field, showed reduced tip kernel abortion, increased number of normal kernels. Transgenic hybrid plants also showed reduced ASI (Anthesis-Silking-Interval) and barrenness rate (percent of the plants without producing the ear). All of these are traits related to abiotic stress tolerance. This is more obvious as the plant density increased from 10,000 to 40, 000 plants per acre, such as the length of the ear cob bearing normal kernel or the number of normal kernels per kernel row.

### Example 30

### ZmARGOS Transgenic Hybrids Stress Tolerance Field Analyses

Field studies with ARGOS8 transgenic hybrids were performed under normal nitrogen, low nitrogen and drought stress across multiple locations. Significant yield increases were seen across each of the stress environments.

A separate set of analyses were performed on hybrid ZmARGOS plants under flowering and grain-filling stress treatments. ZmARGOS8 showed overall positive effects on yield with no particular patterns of interaction with the environments.

Plant height of transgenic ARGOS1 hybrid plants was measured at five stages, starting from V6 to maturity. Transgenic plant showed increased plant height during the growing season, but no difference at maturity, therefore exhibiting faster growth rate. This differs from the Arabidopsis ARGOS gene, where the enhanced plant and organ growth was due to an extended growth period. Transgene expression was quantified from T3 inbred plants sampled from the field by quantitative RT-PCR. A significant correlation was observed between transgene expression and primary ear dry mass of the T2 plants.

### Example 31

### Greenhouse Analyses for ZmARGOS1 for Increased Plant Growth

Two individual events were grown in the greenhouse and the plants were characterized for the number and length. No significant differences in the number of internodes between transgenic plants and control plants. Internode length was measured by the distance between nodes, with the brace roots considered the first node, and the base of the tassel the final node.

Data from two individual events showed that the increased leaf or organ size is primarily due to the increased cell number not cell size. The enhanced cell proliferation is also shown as uneven outgrowth on the leaf epidermis. Therefore, overexpression of ZmARGOS gene promotes plant and organ growth via promotion of cell division.

Transgenic inbred plants overexpressing ZmARGOS1 were characterized at the T2 generation for effects on growth. Plant growth measurements show that the inbred plants have increased plant height, stalk diameter, ear and kernel grown as well as increased primary ear size and rate of producing the secondary ear—an indication of enhanced growth and vigor. Transgenic expression was quantified in T3 inbred plants sampled from field by quantitative RT-PCR. Significant correlation of the transgene expression and the R2 stage secondary ear dry mass was observed.

### Example 32

### In Situ ZmARGOS1 Analyses

In Situ hybridization of maize kernel tissue showed that ZmARGOS1 is expressed in the pedicel. ZmARGOS3 was also detected in the pedicel by MPSS RNA profiling. These data are consistent with the improved grain filling and reduced tip kernel abortion observed in transgenic maize hybrids overexpressing ZmARGOS1. Overexpressing ZmARGOS1 showed a reduction in IAA content as compared to the control, consisted with involvement of auxin regulation in the ARGOS gene function as reported in Arabidopsis.

### Example 33

### The ZmARGOS1 Transgene Affects Yield and Exhibits Transgene x Environment Interaction

Extensive yield trials were conducted to test maize hybrids overexpressing the ZmARGOS1 gene. Yield trial data across multiple locations and years showed that ZmARGOS1 transgenic hybrids exhibited significant yield increase as compared to the control, under specific environment classification including drought stressed environments. In depth analysis of transgene x environment interaction in yield to understand the different performance of the ZmARGOS1 transgenic hybrid in different weather classifications. Weather data (including rain fall, temperature and solar radiation) were collected across locations where yield trials were conducted for each growing season, based upon which the yield trial location was classified to weather categories for each season. Based upon the yield performance and weather data, the ZmARGOS1 transgenic hybrid exhibited significant yield increase under environments with high temperature, less rain fall and high solar radiation. It also showed positive effect on yield under drought stress treatment, both flowering and grain filling stress. However, the transgene has no yield increase or a negative effect on yield under over wet and cool growing conditions. Interaction of genotype by environment (G×E) is a well-recognized phenomenon in crop performance. The data however, provides evidence that a single transgene (ZmARGOS1) has effects on yield interaction with specific environment or weather classification. In addition, the G×E data indicated and support the drought stress tolerance effects of this transgene.

### Example 34

### ZmARGOS8 Transgenic Hybrids Increasesd Yield Under Normal Nitrogen and Low Nitrogen Conditions

Nine ZmARGOS8 transgenic events were tested in field at multiple normal nitrogen locations and multiple low nitrogen locations with 4-6 replicates per location for two years. The second year field testing was expanded to 3 genetic backgrounds. Overall yield testing indicated that 7 out of 9 events showed significant increase in grain yield under normal N conditions with an average 3.0 bushel per acre yield advantage over control at p<0.1 for two years. All nine events had a significant increase in grain yield under low N conditions with an average 2.4 bushel per acre yield advantage over control.

### Example 35

### ZmARGOS8 Transgenic Hybrids Improved Yield Components Under Normal Nitrogen Conditions

To understand the yield advantage of ZmARGOS8 transgene, three individual events were grown in field under normal nitrogen conditions and ear related traits were characterized. Two out of three events showed significant increase in seed weight per ear and kernel numbers per ear compared to their non-transgenic siblings.

In a separate field observation experiment, the ear growth rate measured from silking to 14 DAS (days after silking) was significant faster in 3 out of 10 transgenic events than controls under normal nitrogen conditions. Significant increase in ear length was also observed in ten transgenic events with an average 1.1 cm advantage over control at p<0.1 level from another normal nitrogen field experiment.

### Example 36

### ZmARGOS8 Transgenic Hybrids Enhanced Plant Growth Under Low Nitrogen Conditions

Previously ZmARGOS8 transgenic plants tested in field under normal growth conditions did not show any negative impacts on agronomic traits. To investigate the effects of ZmARGOS8 transgene on plant growth under low N conditions, three individual events were grown in 10 liter pots with 2 mM nitrate treatment in the field and the plants were characterized at V7 and R3 developmental stages for plant biomass. Eight plants per event were sampled and fresh weight of shoot and root was collected. All examined three events showed significant increase in shoot and root biomass at V7 and R3 stages compared to the controls which indicated that ZmARGOS8 transgene improved source capacity via enhancing plant growth under limited nitrogen conditions (FIG. 22).

In a separate experiment, the ARGOS8 transgenic plants tended to have reduced stomata conductance and reduced photosynthesis under different N conditions. The 5% significant reduction on photosynthesis and stomata conductance was only obtained from the event with strongest expression of ARGOS8 transgene at p<0.1 level.

### Example 37

### ZmARGOS8 Transgene Enhanced Root Growth Under Normal Nitrogen and Low Nitrogen Conditions

Three individual events were grown in pots filled with Turface with either 2 mM nitrate or 6 mM nitrate treatment in greenhouse and the roots were harvested at V12 stage for crown root angle measurement. Three plants per event and 4 crown root angles per plant were measured. One event under 6 mM nitrate conditions and all three events under 2 mM nitrate conditions had enlarged crown root angles compared to controls with an average ˜15% increase at p<0.05 (T-test).

In tall tube root assay experiments, two transgenic events and controls were characterized at V5-6 stage for root growth under low nitrate conditions or normal nitrogen conditions. Thirty-two to 40 images of individual whole root system were taken and total images taken from five plants per event at five days, e.g. 10, 14, 17, 21 and 23 days after planting, were analyzed for total root length. The root growth difference was also calculated. The data indicated that two ZmARGOS8 transgenic events had more root biomass represented by total root length and deeper and faster root growth compared to control plants under both normal N and low N conditions. The root system of transgenic plants reached the deeper soil, e.g. ˜4 ft below the surface, 2-3 days earlier than controls and near doubled total root length was observed at this level under normal N conditions. The data are consistent with the root biomass increase under low N conditions (Example 36).

The root plate assay under high N (8 mM nitrate) and low N (1 mM nitrate) conditions was also performed on Arabidopsis lines over-expressing 35S:ZmARGOS8. Increased root biomass was consistently observed from ZmARGOS8 transgenic lines compared to the controls with ˜15% increase in average across 32 reps per treatment under both low N and high N conditions.

### Example 38

### ZmARGOS8 Transgene Increased Cell Numbers/Cell Size

Two individual events were grown in green house under normal nitrogen conditions. The middle part of V6 leaf blades was sectioned, stained and imaged by electron microscopy. The numbers of mesophyll cells were counted. The leaf blades of both transgenic events had ˜10% more cells than those of non-transgenic siblings. The data indicates that ZmARGOS8 transgene enhances organ size via promotion of cell division. However, the leaf blades from one event with higher ZmARGOS8 transgene expression were also ˜25% thicker compared to the null which implied that stronger expression of ZmARGOS8 transgene might enhance not only cell numbers but also cell size.

### Example 39

### Greenhouse ZmARGOS1 Drought Analysis

Greenhouse experiments were conducted to test how shoot growth and root growth were affected by over-expressing ZmARGOS1 in corn plants under drought, well-watered conditions, or water logging. The experiment design was randomized complete block within each treatment. Over-expression of ZmARGOS1 enhanced shoot growth under drought and well-watered condition, in particular. Transgenic plants increased shoot fresh weight by 6.7% and 5.3% under drought and well-watered conditions, respectively. Over expression of ZmARGOS1 in corn enhanced shoot dry weight by 0.8%, 1.1% and 3.4% under water logging, drought and well-watered conditions, respectively. Transgenic corn plants also showed improved water status in plant under drought condition. Positive plants showed higher water content (3.8%) than null.

Over expression of ZmARGOS1 also enhanced root growth under well-watered condition. Root dry weight increased by 10.4% in transgenic event as compared to non-transgenic control.

TABLE 3

| | | | Pos | | | Plant water | |
|---|---|---|---|---|---|---|---|
| | | | or | Shoot FW | Shoot DW | content | Root DW |
| ID | Event # | Treatment | Neg | (g/plant) | (g/plant) | (%) | (g/plant) |
| UBI:ZmARGOS1 | 30.1.3 | Water | Null | NT | 57.59 ± 1.43 | NT | NT |
| | | logging | Pos | NT | 58.04 ± 1.71 | NT | NT |
| | | DRT | Null | 90.38 ± 2.59 | 41.94 ± 0.25 | 53.20 ± 1.09 | NT |
| | | | Pos | 96.44 ± 3.75 | 42.42 ± 0.27 | 55.23 ± 1.47 | NT |
| | | WW | Null | 275.72 ± 9.64 | 58.10 ± 1.09 | 78.72 ± 0.48 | 8.52 ± 1.46 |
| | | | Pos | 290.35 ± 11.41 | 60.07 ± 1.24 | 79.02 ± 0.56 | 9.42 ± 1.20 |

Note:
NT = not tested.
Experiment was conducted in Greenhouse B2 in October, 2011.

### Example 40

### ARGOS Affects the Kernel Number Per Ear and Ear Sizes

Effects of ARGOS over-expression on maize ears and kernels were determined using transgenic plants grown under field conditions. T hree ARGOS constructs, Ubi::ZmARGOS1, Ubi::ZmARGOS5 and Ubi::ZmARGOS8 were planted out as pairs of transgenic events and corresponding non-transgenic controls, five events per construct. Each plot had two rows and the experiment had three replicates. Ear photometry was conducted with ten ears per plot harvested from the middle of the rows. Overexpression of ZmARGOS1, ZmARGOS5 and ZmARGOS8 significantly increased the kernel number per ear by 7.1%, 7.6% and 3.8%, respectively (Table 4). The larger number of kernels in the transgenic ears is mainly due to an increase in ear ring counts. This result is in agreement with the increased kernel count per row, estimated based on the measurement of the ear length and average kernel width. No significant difference in kernel weights and kernel sizes was observed between transgenic plants and non-transgenic controls (Table 4). Ear sizes were larger in two ARGOS constructs; the ear area in ZmARGOS5 and ZmARGOS8 was increased by 6.4% and 3.4%, respectively.

TABLE 4

| | | Mean | Mean | StDev | StDev | Difference | T-test |
|---|---|---|---|---|---|---|---|
| Measurement | Construct | Transgenic | Non-trans | Transgenic | Non-trans | (%) | P value |
| Kernel number | Ubi::ZmARGOS1 | 539.32 | 503.68 | 28.32 | 30.99 | 7.1 | 0.0080 |
| per ear | | | | | | | |
| | Ubi::ZmARGOS5 | 535.84 | 498.01 | 19.52 | 24.12 | 7.6 | 0.0001 |
| | Ubi::ZmARGOS8 | 524.66 | 505.29 | 29.83 | 21.08 | 3.8 | 0.0879 |
| Ear ring count | Ubi::ZmARGOS1 | 36.43 | 34.95 | 1.63 | 1.64 | 4.3 | 0.0331 |
| | Ubi::ZmARGOS5 | 36.41 | 34.45 | 1.15 | 1.33 | 5.7 | 0.0005 |
| | Ubi::ZmARGOS8 | 35.96 | 35.12 | 1.81 | 1.20 | 2.4 | 0.1499 |
| Kernels per row | Ubi::ZmARGOS1 | 33.61 | 32.62 | 1.54 | 1.69 | 3.0 | 0.1465 |
| | Ubi::ZmARGOS5 | 33.87 | 32.13 | 1.00 | 1.30 | 5.4 | 0.0010 |
| | Ubi::ZmARGOS8 | 33.49 | 32.69 | 1.80 | 1.20 | 2.4 | 0.1620 |
| Ear area (cm2) | Ubi::ZmARGOS1 | 76.25 | 73.99 | 3.82 | 4.89 | 3.1 | 0.2260 |
| | Ubi::ZmARGOS5 | 77.01 | 72.38 | 2.68 | 3.42 | 6.4 | 0.0015 |
| | Ubi::ZmARGOS8 | 75.87 | 73.40 | 4.19 | 3.23 | 3.4 | 0.0725 |
| Average single | Ubi::ZmARGOS1 | 0.2626 | 0.2679 | 0.0090 | 0.0131 | −2.0 | 0.2523 |
| kernel weight (g) | | | | | | | |
| | Ubi::ZmARGOS5 | 0.2665 | 0.2610 | 0.0078 | 0.0094 | 2.1 | 0.1253 |
| | Ubi::ZmARGOS8 | 0.2671 | 0.2634 | 0.0122 | 0.0102 | 1.4 | 0.2855 |
| Average kernel | Ubi::ZmARGOS1 | 2.38 | 2.41 | 0.03 | 0.03 | −1.2 | 0.0038 |
| perimeter (cm) | | | | | | | |
| | Ubi::ZmARGOS5 | 2.39 | 2.40 | 0.03 | 0.03 | −0.1 | 0.7006 |
| | Ubi::ZmARGOS8 | 2.40 | 2.40 | 0.03 | 0.04 | −0.2 | 0.6623 |

### Example 41

### Over-Expression of ZmARGOS Improves Drought Tolerance in Arabidopsis Plants.

Transgenic Arabidopsis plants of 35S::ZmARGOS5, 35S::ZmARGOS8 and35S::AtARL3 were tested for drought tolerance. Three events per construct were evaluated with the drought assay, as described below. Arabidopsis plant growth was slowed down when subjected to drought stresses, and the leaves gradually lost chlorophyll and turned yellow. In the drought assay, the transgenic plants over-expressing ZmARGOS5, ZmARGO8 and AtARGOS3 showed significant delay in the yellow color accumulation relative to non-transgenic controls (Table 5). ZmARGOS5, ZmARGOS8 and AtARGOS3 conferred ethylene insensitivity in the Arabidopsis plants. The transgenic Arabidopsis over-expressing a mutated version of ZmARGOS8 [ZmARGOS8(L67D)], in which the 67^{th} amino acid residue leucine in the proline-rich motif was substituted with aspartic acid, had normal ethylene responses and the plants were found not tolerant to the drought treatment (Table 5).

TABLE 5

| | | | Score | |
|---|---|---|---|---|
| Gene | Promoter | Event | (2 sigma) | Deviation |
| AtARGOS3 | 35S | E1 | 8.309 | 26.541 |
| AtARGOS3 | 35S | E2 | 3.554 | 11.903 |
| AtARGOS3 | 35S | E3 | 2.896 | 9.92 |
| ZmARGOS5 | 35S | E1 | 6.769 | 22.399 |
| ZmARGOS5 | 35S | E2 | 5.473 | 18.375 |
| ZmARGOS5 | 35S | E3 | 2.35 | 8.106 |
| ZmARGOS8 | 35S | E1 | 2.572 | 8.752 |
| ZmARGOS8 | 35S | E2 | 2.501 | 8.359 |
| ZmARGOS8 | 35S | E1 | 0.488 | 1.479 |
| (L67D) | | | | |
| ZmARGOS8 | 35S | E2 | 0.344 | 1.055 |
| (L67D) | | | | |
| ZmARGOS8 | 35S | E3 | 0.719 | 0.244 |
| (L67D) | | | | |

Quantitative Drought Assay: 36 glufosinate resistant T2 plants and 36 control plants are sown, each in a single flat on Scotts® Metro-Mix® 360 soil. Flats are configured with 8 square pots each. Each of the square pots is filled to the top with soil. Each pot (or cell) is sown to produce 9 seedlings in a 3×3 array. Within a flat, 4 pots consist of glufosinate resistant plants and 4 pots consist of control plants.

The soil is watered to saturation and then plants are grown under standard conditions (i.e., 16 hour light, 8 hour dark cycle; 22° C.; ˜60% relative humidity). No additional water is given.

Digital images of the plants are taken at the onset of visible drought stress symptoms. Images are taken once a day (at the same time of day), until the plants appear dessicated. Typically, four consecutive days of data is captured.

Color analysis is employed for identifying potential drought tolerant lines. Color analysis can be used to measure the increase in the percentage of leaf area that falls into a yellow color bin. Using hue, saturation and intensity data (“HSI”), the yellow color bin consists of hues 35 to 45.

Maintenance of leaf area is also used as another criterion for identifying potential drought tolerant lines, since Arabidopsis leaves wilt during drought stress. Maintenance of leaf area can be measured as reduction of rosette leaf area over time.

Leaf area is measured in terms of the number of green pixels obtained using an imaging system. Transgenic and control (e.g., wild-type) plants are grown side by side in flats that contain 72 plants (9 plants/pot). When wilting begins, images are measured for a number of days to monitor the wilting process. From these data wilting profiles are determined based on the green pixel counts obtained over four consecutive days for transgenic and accompanying control plants. The profile is selected from a series of measurements over the four day period that gives the largest degree of wilting. The ability to withstand drought is measured by the tendency of transgenic plants to resist wilting compared to control plants.

Estimates of the leaf area of the Arabidopsis plants are obtained in terms of the number of green pixels. The data for each image is averaged to obtain estimates of mean and standard deviation for the green pixel counts for transgenic and wild-type plants. Parameters for a noise function are obtained by straight line regression of the squared deviation versus the mean pixel count using data for all images in a batch. Error estimates for the mean pixel count data are calculated using the fit parameters for the noise function. The mean pixel counts for transgenic and wild-type plants are summed to obtain an assessment of the overall leaf area for each image. The four-day interval with maximal wilting is obtained by selecting the interval that corresponds to the maximum difference in plant growth. The individual wilting responses of the transgenic and wild-type plants are obtained by normalization of the data using the value of the green pixel count of the first day in the interval. The drought tolerance of the transgenic plant compared to the wild-type plant is scored by summing the weighted difference between the wilting response of transgenic plants and wild-type plants over day two to day four; the weights are estimated by propagating the error in the data. A positive drought tolerance score corresponds to a transgenic plant with slower wilting compared to the wild-type plant. Significance of the difference in wilting response between transgenic and wild-type plants is obtained from the weighted sum of the squared deviations.

Lines with a significant delay in yellow color accumulation and/or with significant maintenance of rosette leaf area, when the transgenic replicates show a significant difference (score of greater than 2) from the control replicates, the line is then considered a validated drought tolerant line.

### Example 42

### Overexpression of Maize ARGOS Affects Ethylene Signaling and Ethylene Responsive Gene Expression in Maize

RNA-seq was used to analyze the expression of ethylene signaling and ethylene responsive genes in transgenic maize plant leaves and null controls. Overexpression of ZmARGOS1 and ZmARGOS5 significantly reduced the transcript levels of ethylene receptor ZmERS1. Expression of ethylene receptor-interacting protein ZmRTE1 and ZmRTE3 was also down-regulated in the ZmARGOS1, ZmARGOS5 and ZmARGOS8 plants. Maize EIN3 is a master transcription factor in ethylene signal transduction pathway and the EIN3 F-box binding protein, ZmEBF1 which regulates EIN3 protein degradation, was found affected by ZmARGOS over-expression. ZmEBF1 mRNA in transgenic leaves was up-regulated in comparison to null controls. The change in the ZmEBF1 transcript levels may result in reduced EIN3 transcriptional activities and consequently altered expression of ethylene responsive genes. As expected, the ethylene responsive factor ZmEREBP1 and ZmERF1 were found down-regulated in ZmARGOS1 and ZmARGOS5 plants while ZmERF2 was up-regulated.

### Example 43

### Over-Expression of Maize ARGOS Genes Improve Maize Yields Under Drought Stresses

Ten UBI:ZmARGOS5 events were evaluated in yield trials conducted under drought stress targeted during flowering and grain-fill. Average yields of the controls under these treatments were 159 bu/acre and 176 bu/acre respectively. Under the flowering stress treatment, six of the ten events showed a significant 8 bu/acre increase in yield relative to the non-transgenic control. The other four events were not significantly different. Under the grain fill stress treatment, five of the ten events showed an average significant increase of 13 bu/acre when compared to the non-transgenic control. Two of the events showed a significant 3 bu/acre decrease, and three events were neutral.

In next year, the top five events were evaluated under the drought testing program again at additional locations. In total, the construct was evaluated in six environments consisting of Site A flowering stress (167 bu/acre), very mild stress Site A (201 bu/acre), Site B (162 bu/acre), Site C (107 bu/acre), Site D (38 bu/acre) and Site E (178 bu/acre). In both the Site A mild stress and the Site C environments, four of the five events showed a significant increase in yield over the non-transgenic control that average 6 bu/acre and 10 bu/acre respectively. In the other environments the effect of the transgene was neutral. In a multi-location analysis, three of the five events showed a significant increase in yield relative to the control that averaged 3 bu/acre.

Transgenic maize plants overexpressing ZmARGOS8 were evaluated under drought stress treatments with various combinations of testers under Site A flowering (WO-FS) and grain fill (WO-GF) as well as a severe stress in Site C (GC-FS). Under WO-FS, UBI:ZmARGOS8 showed a 4.3 bu/acre and 6.0 bu/acre increase relative to the bulk null with HNH9HBH2 and GR1B5B9 testers respectively. No other tester x location combination was significantly different than the bulk null at the construct level. The event was also evaluated under low and normal nitrogen. Across all low N environments, the construct mean was 2 bu/acre greater than the bulk null which was significant at P<0.10.

A multi-year analysis (2009-2010) identified 8 of the 10 events as having a significant increase in yield relative to the control. These advantages ranged from 1.7 bu/acre to 2.9 bu/acre (FIG. 23).

### Example 44

### ZmArgos1 Transgene Effect on Root Growth and Leaf Area in Different Genetic Backgrounds and Yield Increase

The experiments involving transgenic maize plants expressing ZmArgos1 were conducted in greenhouse in plexiglass chambers. Plants were harvested when 5-6 leaves were fully expanded, root systems were washed and transferred to a metallic grid where they were imaged using a digital camera. Leaf area was measured for each plant. Leaves, roots and stems and sheaths were dried to constant weight. Two transgenic and non-transgenic pairs and analyses were conducted by pair. Ratio between width and depth (the higher the ratio the more rectangular the root system) of the roots and the root angle were measured among other traits.

The ZmArgos1 transgene affected growth in one of the two genetic backgrounds tested. In the other genetic background, the expression of the transgene affected root angle and width-to-length ratio in. Similarly, in one of the genetic backgrounds, the transgene increased leaf expansion (+480 cm2+/−106; df=15; P<0.05), leaf biomass (+1.7 g+/−0.4; df=15; P<0.05) and total above ground biomass (+3.1 g+/−0.7; df=15; P<0.05). Increase in leaf area and biomass were such that specific leaf are (cm2/g) remained constant. In contrast, in this genetic background, the transgene did not affect root growth significantly and no significant difference was detected in the root biomass (+1.4g+/−2.1; df=15). In the second genetic background, the effects of the transgene were evident and significant on root angle (−9.2 degrees+/−2.9; df=15; P<0.05) and width to length ratio (+0.015+/−0.006; df=15; P<0.05). For a given depth the root system of the transgenic plant was wider that the non-transgenic (Null).

Results from this experiment indicate two possible mechanisms by which the transgene can affect yield in maize plants: (a) Water use pattern affected by changes in leaf area development (b) Water capture via effects on root angle and width-to-length ratio (c) Growth and (d) Allocation of growth to above ground biomass, when the harvest index remains constant increase biomass production translates into increase yield. Harvest index depends on severity of environmental stress and crop management.

### Example 45

### Variants of ARGOS Sequences

A. Variant Nucleotide Sequences of ARGOS That Do Not Alter the Encoded Amino Acid Sequence

The ARGOS nucleotide sequences are used to generate variant nucleotide sequences having the nucleotide sequence of the open reading frame with about 70%, 75%, 80%, 85%, 90% and 95% nucleotide sequence identity when compared to the starting unaltered ORF nucleotide sequence of the corresponding SEQ ID NO. These functional variants are generated using a standard codon table. While the nucleotide sequence of the variants are altered, the amino acid sequence encoded by the open reading frames do not change.

B. Variant Amino Acid Sequences of ARGOS Polypeptides

Variant amino acid sequences of the ARGOS polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using the protein alignment set forth in FIGS. 2, 12 and 21, an appropriate amino acid can be changed. Once the targeted amino acid is identified, the procedure outlined in the following section C is followed. Variants having about 70%, 75%, 80%, 85%, 90% and 95% nucleic acid sequence identity are generated using this method.

C. Additional Variant Amino Acid Sequences of ARGOS Polypeptides

In this example, artificial protein sequences are created having 80%, 85%, 90% and 95% identity relative to the reference protein sequence. This latter effort requires identifying conserved and variable regions from the alignment set forth in FIGS. 2, 12 and 21 and then the judicious application of an amino acid substitutions table. These parts will be discussed in more detail below.

Largely, the determination of which amino acid sequences are altered is made based on the conserved regions among ARGOS protein or among the other ARGOS polypeptides. Based on the sequence alignment, the various regions of the ARGOS polypeptide that can likely be altered are represented in lower case letters, while the conserved regions are represented by capital letters. It is recognized that conservative substitutions can be made in the conserved regions below without altering function. In addition, one of skill will understand that functional variants of the ARGOS sequence of the disclosure can have minor non-conserved amino acid alterations in the conserved domain.

Artificial protein sequences are then created that are different from the original in the intervals of 80-85%, 85-90%, 90-95% and 95-100% identity. Midpoints of these intervals are targeted, with liberal latitude of plus or minus 1%, for example. The amino acids substitutions will be effected by a custom Perl script. The substitution table is provided below in Table 6.

TABLE 6
Substitution Table

| | Strongly | | |
|---|---|---|---|
| | Similar and | Rank of | |
| | Optimal | Order to | |
| Amino Acid | Substitution | Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C and P are not changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involve a 50:50 substitution of the two alternate optimal substitutions.

The variant amino acid sequences are written as output. Pert script is used to calculate the percent identities. Using this procedure, variants of the ARGOS polypeptides are generating having about 80%, 85%, 90% and 95% amino acid identity to the starting unaltered ORF nucleotide sequence of SEQ ID NOS: 1-37, 40-91 and 96-102.

All publications and patent applications in this specification are indicative of the level of ordinary skill in the art to which this disclosure pertains. All publications and patent applications are herein incorporated by reference to the same extent as if each individual publication or patent application was specifically and individually indicated by reference.

The disclosure has been described with reference to various specific and preferred embodiments and techniques. However, it should be understood that many variations and modifications may be made while remaining within the spirit and scope of the disclosure.

What is claimed is:

1. A method of modulating the ethylene sensitivity in a plant, comprising:
a introducing into a plant cell a recombinant construct comprising a polynucleotide encoding a transmembrane protein comprising a proline rich motif having a sequence PPLXPPPX (SEQ ID NO: 96),wherein the proline rich domain is located between a first transmembrane sequence and a second transmembrane sequence, operably linked to a promoter; and
b. expressing said polynucleotide to modulate the level of ethylene sensitivity in said plant.

2. The method of claim 1, wherein the proline rich motif (PRM) sequence comprises:
a. original PRM (SEQ ID NO: 88), or
b. variant PRM (SEQ ID NO: 102)

3. The method of claim 1, wherein the plant is selected from the group consisting of: maize, soybean, sorghum, canola, wheat, alfalfa, cotton, rice, barley, millet, peanut, sugarcane, miscanthus, poaceae, cocoa, camelina, 1pomoea and Solanum.

4. A method of modulating the ethylene sensitivity in a plant, comprising:
a. introducing into a plant cell a nucleotide construct comprising a polynucleotide which encodes a TPT domain having at least 50% sequence identity to that of TM1 SEQ ID NO: 90 or TM2 SEQ ID NO: 91 operably linked to a promoter, also including the proline motif of claim 2; and
b. growing the plant under either a drought or a low nitrogen condition.

5. The method of claim 4, wherein the plant is selected from the group consisting of: maize, soybean, sorghum, canola, wheat, alfalfa, cotton, rice, barley, millet, peanut, sugarcane, poaceae, cocoa, camelina, 1pomoea and Solanum.

6. The method of claim 5, wherein said plant cell is from a monocot.

7. The method of claim 6, wherein the plant cell is from maize.

8. The method of claim 1 wherein the ethylene sensitivity is decreased.

9. The method of claim 1 wherein said construct is an over expression construct.

10. The method of claim 1 wherein said construct comprises SEQ ID NO: 88 or SEQ ID NO: 102.

11. A transgenic plant produced by the method of claim 1.

12. The transgenic plant of claim 1, wherein the plant has decreased ethylene sensitivity when compared to a plant which has not been transformed.

13. The transgenic plant of claim 1, wherein the plant has decreased susceptibility to abiotic stress.

14. The transgenic plant of claim 11 wherein the plant has decreased susceptibility to drought stress.

15. The transgenic plant of claim 11, wherein the plant has decreased susceptibility to crowding stress.

16. The transgenic plant of claim 11, wherein the plant has decreased susceptibility to flooding stress.

17. An isolated protein comprising a member selected from the group consisting of:
a. polypeptide of at least 20 contiguous amino acids from a polypeptide of SEQ ID NO: 89;
b. a polypeptide of SEQ ID NO: 89;
c. a polypeptide having at least 80% sequence identity to, and having at least one linear epitope in common with, a polypeptide of SEQ ID NO: 89, wherein said sequence identity is determined using BLAST 2.0 under default parameters; and,
d. at least one polypeptide encoded by a member of claim 1.

18. An isolated polynucleotide sequence encoding a protein with ethylene regulatory activity having the sequence of SEQ ID NO: 89.

19. A polypeptide with ethylene regulatory activity having the sequence of SEQ ID NO: 89.

20. A method of increasing yield in a crop plant, the method comprising
a. expressing a recombinant construct comprising a polynucleotide encoding a transmembrane protein comprising a proline rich motif having a sequence PPLXPPPX (SEQ ID NO: 96), wherein the proline rich domain is located between a first transmembrane sequence and a second transmembrane sequence, operably linked to a promoter; and
b. increasing the yield of the crop plant, wherein the yield is increased under lower than normal nitrogen levels.

21. The method of claim 20, wherein the lower nitrogen level is about 10% to about 40% less compared to a normal nitrogen level.

22. The method of claim 20, wherein the crop plant is maize.

23. The method of claim 22, wherein the maize is hybrid maize.

24. A method of improving an agronomic parameter of a maize plant, the method comprising
a. expressing a recombinant construct comprising a polynucleotide encoding a transmembrane protein comprising a proline rich motif having a sequence PPLXPPPX (SEQ ID NO: 96), wherein the proline rich domain is located between a first transmembrane sequence and a second transmembrane sequence, operably linked to a promoter; and
b. improving at least one of the agronomic parameters selected from the group consisting of root growth, shoot biomass, root biomass, kernel number, ear size, and drought stress.

25. The method of claim 22, wherein the agronomic parameter is improved under low nitrogen levels.

26. A method of marker-assisted selection of a maize plant that exhibits an altered expression pattern of an endogenous gene, the method comprising:
a. obtaining a maize plant comprising an allelic variation in the genomic region of a polynucleotide encoding a transmembrane protein comprising a proline rich motif having a sequence PPLXPPPX (SEQ ID NO: 96), wherein the expression of the polynucleotide is increased compared to a control maize plant not having the variation;
b. selecting the maize plant comprising the variation; and
c. developing a population of maize plants comprising the variation through marker-assisted selection process.

27. The method of claim 26, wherein the variation is present in the regulatory region of the genomic region.

28. The method of claim 26, wherein the variation is present in the coding region of the polynucleotide.

29. The method of claim 26, wherein the variation is present in the non-coding region of the genomic region.

30. The method of claim 26, wherein the expression of the polynucleotide is increased differentially in different genetic backgrounds.


SEQUENCE LISTING (104 sequences)

SEQ ID NO: 1 - DNA, 879 bp, Zea mays
tttttagctagctagatctggcctgattcgccgatcgagcggtggtgagacggagtgcttcagctcaaagactgctagtggtaggctggtagctagctgtgtgcctgtgtgcagtgtgcactgccactgcatgcgcggcgccttggacttaagacggcagcacacgcacgcgaggaggcgtcggctgaagcgagcgctccggcggctccgcttcgctcatcaggttcttgagccccggaaacgatgagcacgacccggccggaggacacccagcaactgatcaacagtgccgccgctagccccaaccgcagcgcaccgtccgccgcgcccagcgatatggagaggggcagcggaaccgccgcgtcctcgtcgcgcgcttcgacgacgtctcactcccaccagagggccacccacagggtggtggaggaggaggaggaggaggagcctagtagcagccgtggcggcggcagcctctgctccgggtacctgtcgctcccggctctgctgctcgtcggcgtcaccgcgtcgctggtgatcctcccgctcgtcctgcccccgctgccgccgccgccgtcgatgctgatgctggtccccgtggcaatgctgctcctgctgctcgtgctggcgttcatgcccacgtcgtccaccggcggccgcggtggaaccggaccgacctacatgtagataatcacatcggttttttttttcctttctttctcttgtcgtcctttcgtttggattttgtgacagagggaggtcttgcgatggatcagttagtcctcagcttctgctcttctcgatcgtacgatgtctctgttcggctaattaatttgcataggggtatatatatgctgcctagatcttaaaagtatctcgtgc

SEQ ID NO: 2 - protein, 146 aa, Zea mays
MSTTRPEDTQQLINSAAASPNRSAPSAAPSDMERGSGTAASSSRASTTSHSHQRATHRVVEEEEEEEPSSSRGGGSLCSGYLSLPALLLVGVTASLVILPLVLPPLPPPPSMLMLVPVAMLLLLLVLAFMPTSSTGGRGGTGPTYM

SEQ ID NO: 3 - DNA, 936 bp, Zea mays
caacgtccaacccctcttgtctctcgtctacctctcttctgcccctctgcgtccgtgtctccctcgtcgtcgctgcgtgaggttgacgacgaccagtcacaggatctgttcgttcctcatgcgacccagctagctaaaactggcatgcatggacatgctacgctgctgcgtcaatccatctcaccagcagtgctagctagctagatctggcctgattcgccgatcgagcggtcgccggtcagagactcagagttcatgagacggagtgcttcagctcaaagactgctagtggtagctaggtagctgcgtgcactgcatgcgcggcgccttggacttgaagaaaccgagcgctccgatagtccgatccggaaacgatgagtgccgggccggaggacacccagcagctgatcaacagtgccgccgctagccccaaccgcagcgcaccgtccgccgcgcccagcgatatggagaggggcagcggaaccgccgcgtcctcgtcgcgcgcttcgacgacgtcccactcccaccagagggccacccacagggtggtggaggaggaggaggaggagcctagtagcagccgtggcgccggcagcctctgctccgggtacctgtcgcttccggctctgctgctcgtcggcgtcaccgcgtcgctggtgatcctcccgctcgtcctgcccccgctgccgccgccgccgtcgttgctgatgctggtccccgtggcaatgctgctcctgctgctcgtgctggcgttcatgcccacgtcgtccaccggcggccgcggtggaaccggaccgacctacatgtagataatcacatcggtttttttttttttcctttctttctcttgtcgtcctttcgtttggattttgtgacagagggaggtcttgcgatggatcagttagtcctcaaaaaaaaaaaaaaaaaaaaaaaaaa

SEQ ID NO: 4 - protein, 144 aa, Zea mays
MSAGPEDTQQLINSAAASPNRSAPSAAPSDMERGSGTAASSSRASTTSHSHQRATHRVVEEEEEEPSSSRGAGSLCSGYLSLPALLLVGVTASLVILPLVLPPLPPPPSLLMLVPVAMLLLLLVLAFMPTSSTGGRGGTGPTYM

SEQ ID NO: 5 - DNA, 1067 bp, Zea mays
ctccatccttccccccgggagcaggagctgcagccaggagtcgagtcggcgtcgtcacgggagatatcagcttcgctatcaccggatcccccctctgctccctccgcacctcccatctgcgctctctgttttcttccgcgcaccccggctgttggtgtcccgtccggcggcgttgctggtggctgaatccgagcctttgaggggtctcccgccgccgccgctcttgagatctctttattgatctggagggattaaagagggattcttgccttcctactggagcaagagaaaggggagaacgtgtttcttcaggcgtggttgaacagtgaggaccggagaacaatgcgaggttcgggatttaagatgttctggctttaggggccgttcttctgaagcaggggacgggcgattcgaccaccggagctcagatctgattacaaaacgttcagaaaacacaaggcgttctcacaccgcctttcacttcttgcttactttggcaaccactcactgcgactggtctccacctccacctacaccaaagaacacatggcaagccgatctagcgcgatggaaggaggggcggcaatacaaaggaggaatgccgtgaagcggcatctgcagcagcgtcagcaggaggcggatttcctcgacaagaaggtcatcgcgtccacctacttcagcatcggggcgttcctcgtgctcgcctgcctcaccgtctcgctgctgatactgccgctggtgctgcctcccctgccgccgccgccgtcgctgctgctgtggctgccggtctgcctgctcgtcttgctggttgtactggccttcatgccgacagatgtgcgcagcatggcctcctcttacctgtaaatagataaataggtcttggccagattttctgtgttttgcagctgcaggattcgtcctaagacgagtcatgagtgtaatgtgaagcaacttctccagggatagatctcaaccaagtttggtagccatacgaagttattgactggaatttagaacatatagttgtgcacaatttcgaacatatcttgtagtggagagcgggccga

SEQ ID NO: 6 - protein, 105 aa, Zea mays
MASRSSAMEGGAAIQRRNAVKRHLQQRQQEADFLDKKVIASTYFSIGAFLVLACLTVSLLILPLVLPPLPPPPSLLLWLPVCLLVLLVVLAFMPTDVRSMASSYL

SEQ ID NO: 7 - protein, 152 aa, Zea mays
MCRGLPTPAPAPALQFQSQDCSRQQRGTTQAPPGRASESVRACMAAERKAASRPAACGRMRGAEGAKPRGRQAKAARAPPGQGYFTAGLAALFLCLTTLLVFLPLVLPPLPPPPLLLLLVPVGLMAVLLALALVPSDGRAAAAAVASSSCVC

SEQ ID NO: 8 - protein, 119 aa, Zea mays
MHLLDDLRQDRGGAAAHTGSRSRKPPPPLAAAAAAAAGVPAGSSTAATATHLGPEAAALLACVTATLLLLPLVLPPLPPPPPLLLLVPVAIFAVLLLLVLLPSDARAAVATPTSSASYL

SEQ ID NO: 9 - protein, 64 aa, Zea mays
MSKRVLMMLLAATVILLCLPLVLPPLPPPPLFLLFVPVVMMLLLFSLVFFPSNHCPCSSPTFTQ

SEQ ID NO: 10 - protein, 106 aa, Zea mays
MPSSSQTPPPPVGRTAAHGGRHKHDDDDPSTPRGFCAKYFSRESCLLLALVTVLLVVLPLVLPPLPAPPLALLLVPVAMLAVLLVLALMPAAAGGRNEAVDPASYL

SEQ ID NO: 11 - protein, 118 aa, Zea mays
MMLHCTFAISEAPARALALGQVSVMRAMPQEEEAAVATTTMAGGKVAALLATAAALLLLLPLALPPLPPPPTQLLFVPVVLLLLVASLAFCPAATSSPSPMHAADHGSFGTTGSPHLC

SEQ ID NO: 12 - protein, 126 aa, Zea mays
MPVASSLMAMELETDQLAWAEQQRQQNRRQTMVVCRKSDAAVAKGQQRQNASPPSPKPPPAGGLSAEAFLVLACVAVSLIVLPLVLPPLSPPPPLLLLVPVCLLLLLAALATFVPSDVRSMPSSNL

SEQ ID NO: 13 - protein, 103 aa, Oryza sativa
MKTTLAVVEGTRAHIVNLANSRASRLNERLIDPAIESRSIAGATPAPFEMETAMVLLLLALVAFLLCYPLVLPPLPPSPPALFIWIPVFMLLLLFALALFPVQ

SEQ ID NO: 14 - protein, 68 aa, Oryza sativa
MVMLLLAAAAVLLLLLPLLLPPLPPPPSLLLLVPVVLLLALLSLAFLPNRDVVVYGQQPAADQFFFRQ

SEQ ID NO: 15 - protein, 147 aa, Oryza sativa
MSFAIRSSEPEFWFLIPSEEAAVAVAAHRLVVMDQRRSGSAYRPKRTHMAAAEDEHRRPGTSSRRRVAPTPTTQTQTQTAPGYFTVELVMAFVCVTASLVLLPLVLPPLPPPPSLLLVVPVCLLAVLVAMAFVPLDAQSNVVGSSCL

SEQ ID NO: 16 - protein, 130 aa, Oryza sativa
MEKGRGKACGGGSTAPPPPPPSSSGKSGGGGGSNIREAAASGGGGGVWGKYFSVESLLLLVCVTASLVILPLVLPPLPPPPSMLMLVPVAMLVLLLALAFMPTTTSSSSSAGGGGGGGRNGATTGHAPYL

SEQ ID NO: 17 - protein, 127 aa, Oryza sativa
MRGVILLRYEEDAMAGHRSTAAATGGRLYGQVGVKRRVVEETAAAVEVGGGGGGYLGVEAAVLLGVVTATLLVLPLLLPPLPPPPPMLLLVPVAIFAVLLLLVLLPSDAKSIAAAGRPSSSSSSSYL

SEQ ID NO: 18 - protein, 105 aa, Oryza sativa
MQEEAASSSSSSASPVMDGGKAMAVLLAVAAAVLLLLPLVLPSLLLLLPVVLLLLVVSLAFFPAAGSDGVVAAAAVAGTYQPPPPPPARSSPPPSSSSSSSSRQL

SEQ ID NO: 19 - protein, 105 aa, Oryza sativa
MEGVGARQRRNPLIPRPNGSKRHLQHQHQPNAAEKKTAATSNYFSIEAFLVLVFLTMSLLILPLVLPPLPPPPSLLLLLPVCLLILLVVLAFMPTDVRSMASSYL

SEQ ID NO: 20 - protein, 120 aa, Oryza sativa
MEEQMFREQQMQRGGRHHQHHTTREQEQQQKQQQRRRLMNNATNGGGGDGGSRCYFSTEAILVLACVTVSLLVLPLILPPLPPPPTLLLLLPVCLLALLVVLAFMPTDMRTMASSYFFCL

SEQ ID NO: 21 - protein, 96 aa, Glycine max
MMMVHPRDQVGGETHKNLVEPNVAASKKARNCACMVSYSVLILALLTLSILLLPLVLPPLPPPPLLLLFVPVFILVVLFFLAFSPSTLPNMAVLTS

SEQ ID NO: 22 - protein, 96 aa, Glycine max
MMMVHPRDQVGGDTHKNLVAPNVAASKKARNCACMVSYSVLILALLTLFILLLPLVLPPLPAPPLLLLFVPVFLLVVLFFLAFSPSTLPNMAVLTS

SEQ ID NO: 23 - protein, 102 aa, Glycine max
MARCFGLGSVLVLAALAASMVVLPLMLPPLPPPPLVLLFFPVGIMAALMLLAFSPSDQNGVVYASTRRWWETGSAGATFWGFLKVPMGLLRFMFFFFFKLRC

SEQ ID NO: 24 - protein, 66 aa, Glycine max
MARCFGLGSVLVLAALAASMVVLPLMLPPLPPPPLVFFFFPVGIMAALMLLVFSPSDQNGVVYATT

SEQ ID NO: 25 - protein, 90 aa, Glycine max
MSSWLIHYNKRFIISISLAFMLRLFGFKSTMFMVVLTIAILVLPLMLPPLPPPPMILMLVPLVIMLLLVKLALYSKHGPADVIYQCNFTW

SEQ ID NO: 26 - protein, 130 aa, Arabidopsis thaliana
MIREISNLQKDIINIQDSYSNNRVMDVGRNNRKNMSFRSSPEKSKQELRRSFSAQKRMMIPANYFSLESLFLLVGLTASLLILPLVLPPLPPPPFMLLLVPIGIMVLLVVLAFMPSSHSNANTDVTCNFM

SEQ ID NO: 27 - protein, 135 aa, Arabidopsis thaliana
MIREFSSLQNDIINIQEHYSLNNNMDVRGDHNRKNTSFRGSAPAPIMGKQELFRTLSSQNSPRRLISASYFSLESMVVLVGLTASLLILPLILPPLPPPPFMLLLIPIGIMVLLMVLAFMPSSNSKHVSSSSTFM

SEQ ID NO: 28 - protein, 88 aa, Arabidopsis thaliana
MRVHDQRLRFDVTPKPMGLNGSSLITARSVALLLFLSLLLLILPPFLPPLPPPPATLLLLPLLLMILLIFLAFSPSNEPSLAVEPLDP

SEQ ID NO: 29 - protein, 146 aa, Sorghum bicolor
MSTGRPEDIQQLINSATSSPNRTSPSASPSDMESGGGSASSPRASTSDRRLQRAAHSHREEWEPAAAASGDGGTGSLWSRYFSLPVLLLVGVTASLVILPLVLPPLPPPPSMLMLVPVAMLVLLLVLAFMPTSSVRAGTGTGPTYM

SEQ ID NO: 30 - protein, 107 aa, Sorghum bicolor
MASRSSALEGGGAAIQRRNNAVKRHLQQRQQEADFHDKKVIASTYFSIGAFLVLACLTFSLLILPLVLPPLPPPPSLLLWLPVCLLVLLVVLAFMPTDVRSVAASYL

SEQ ID NO: 31 - protein, 88 aa, Sorghum bicolor
NAVKRHLQQRQQEADFHDKKVIASTYFSIGAFLVLACLTFSLLILPLVLPPLPPPPSLLLWLPVCLLVLLVVLAFMPTDVRSMASSYL

SEQ ID NO: 32 - protein, 58 aa, Sorghum bicolor
MMLLVATVILLCLPLVLPPLPPPPLFLLFVPVVMMLLLFSLVLFPSHHCACSSPTFTQ

SEQ ID NO: 33 - protein, 148 aa, Sorghum bicolor
MSFVAGSSEADQLWFLIPSEQARAHAVQPHHPLAMDRRSSARRRGDPHPHRRGAMHGAAEQQKQQQQRGRPQGTRAAPPVPPGYFTAELVLAFLFVAVSLAFLPLVLPPLSPPPFLLLLVPVGLLAVLLALAFVPLDAHSHLVVGSSR

SEQ ID NO: 34 - protein, 120 aa, Sorghum bicolor
MAEERKQAGSRWPAGGSGGGRMRDAEGGSGKMRGRQATKARPVVLAPPGQGYFTAGLAALFLCLTALLVFLPLVLPPLPPPPYLLLLVPVGLMAVLLALVALVPSDGRAATAAVASSCVC

SEQ ID NO: 35 - protein, 101 aa, Sorghum bicolor
MRRAVPQEEAVAAATTTTMDGGKVVALLATAAALLLLLPLALPPLPPPPTQLLFVPVVMLLLVASLAFCPTAASSGGGGKSKLADADHGSSFRTTGSPHLR

SEQ ID NO: 36 - protein, 106 aa, Sorghum bicolor
MPSPSQTSPPVGRRTAHGGWHKHDDPSTPRGFCTKYFSVESCLLLALVAVLLLVLPLVLPPLPPPPLAVLLVPVAMLAVLLVLALMPVAAAAAGARNEVVDPASYL

SEQ ID NO: 37 - protein, 72 aa, Sorghum bicolor
MERSMVTMLLLATAAVVLLLLPLLLPSSLPPPPSLLLVVPVVLLLSLLSLAFLPTRDDDDAIAIYGSLRSVQ

SEQ ID NO: 38 - DNA, 13 bp, Artificial sequence
  primer
cgctagccccaac

SEQ ID NO: 39 - DNA, 27 bp, Artificial sequence
  primer
cacataacacacaactttgatgcccac

SEQ ID NO: 40 - DNA, 459 bp, Zea mays
atgtgccgcggcctcccaactccagctccagctccagcgcttcaatttcagtcccaggattgcagtcggcagcagcgaggtactacccaagcaccgcccggccgagcgagcgagtccgtgcgtgcgtgcatggcagcagagaggaaggcggcctcccgcccggccgcctgcgggcgaatgcgcggcgccgagggtgccaagccgcggggccgtcaggcaaaggcagcgcgggcaccaccgggccaggggtacttcacggcggggctggcggcgctgttcctttgcctcaccacgctgctcgtgttcctgcctctcgtgctgccgccgctgccgccgccgccgttgctgctgctgctcgtgcccgtgggcctcatggctgtactgcttgcgctggcgctcgtgccgtccgacggccgggccgccgccgccgccgtcgcttcttcatcgtgcgtgtgctga

SEQ ID NO: 41 - DNA, 360 bp, Zea mays
atgcacctgctcgacgacctccgccaagaccgcggcggcgcggccgcccacaccggcagccgcagtcgcaagccgcccccgccccttgccgccgccgccgccgccgccgcgggggtcccggcgggctcctccaccgccgccaccgccacccacctgggcccggaggcggcggcgctgctggcgtgcgtcacggccacgctgctgctgcttccgctggtcctgccgcccctgccgccgccgccgccgctcctcctcctcgtgcccgtcgccatcttcgccgtcctgctactcctcgtgctcctcccctccgacgcccgcgccgccgtcgccacgcccacctcctccgcctcctacttgtag

SEQ ID NO: 42 - DNA, 195 bp, Zea mays
atgagcaagagagtactgatgatgttgctggcggcgacagtgatcctcctgtgcctgccgttggtgctgccaccccttccgccaccaccgctgtttcttctcttcgtccctgtggtgatgatgctcctgctcttctccctggttttcttcccgtctaaccactgtccatgctcttctccgaccttcactcagtaa

SEQ ID NO: 43 - DNA, 321 bp, Zea mays
atgccgtcatcgtcgcagacaccgccgccgccggtcgggaggactgctgctcacggcggccggcacaagcacgacgatgacgacccaagcacgccgaggggcttctgcgccaagtacttctccagggagtcgtgcctcctgctcgccctcgtcaccgtgctgctggtggtgctcccgctcgtcctgccgccgctcccggcgccgccgttggcgctgctgctcgtgccggtcgcaatgttggcggtgctgctggtgctcgcgctcatgccggcggcggcaggtggccggaacgaggctgtggacccggcgtcgtacttgtag

SEQ ID NO: 44 - DNA, 357 bp, Zea mays
atgatgctgcactgcacatttgctatatctgaggctcctgcgcgcgccttggcccttggccaggtgtctgtcatgcgggcgatgccgcaggaagaagaagccgcggtggcgacgacgaccatggccgggggcaaggtggcggcgctgctggccacggcggccgcgctgctgctgctgctcccgctggcgctgccgccgctgccgccgccgcccacgcagctgttgttcgtccccgtggtcttgctgctcctcgtggcgtccctcgcgttctgccccgccgcgacctcctcgccgtcgccgatgcatgccgccgaccacgggtcgttcgggaccactggatcaccgcacctatgttga

SEQ ID NO: 45 - DNA, 381 bp, Zea mays
atgccggttgcttcgtcgctaatggcgatggagttggagacggaccaactcgcctgggcggagcagcagcggcagcagaataggaggcagaccatggtcgtctgcagaaagagcgacgcagcggtggccaaagggcagcagcgtcagaacgcttcgccgccgtcgcccaagcctccgcccgcgggcgggctcagcgcggaggcgttcttggttctggcgtgcgtcgccgtgtcgctcatcgtgctgccgctggtcctgccgccgctgtcgcccccgccgcctctgctgctgctggtgccggtgtgcctgctcctgctcctcgccgcgctcgccaccttcgtgccgtcggatgtcaggagcatgccatcctccaacttgtaa

SEQ ID NO: 46 - DNA, 312 bp, Oryza sativa
atgaagacgactttggctgtggtggaagggaccagggcacatattgttaacctggcgaattcaagggcgtctcgattgaacgaacggctgatcgatccagcaatcgagtctcgatcgattgccggagcaacacctgcgccgtttgagatggagacggcaatggtgctgctgctgcttgcactggtcgccttccttctctgctaccctcttgttctaccaccgctgccgccttcgcccccggccctgttcatctggataccggtgttcatgctgctcctgctcttcgcccttgccctcttccctgttcagtaa

SEQ ID NO: 47 - DNA, 207 bp, Oryza sativa
atggtgatgcttctcctcgctgcggcggcggtgctgctgctgctgctcccgctgctgctcccgccgctgccgccgccgccgtcgctgctgctgctcgtccccgtcgtgctgctgctggcgctcctttccctcgctttcctccccaaccgcgacgtcgtcgtctacggacagcagccagctgcggatcaattcttcttccgacaatga

SEQ ID NO: 48 - DNA, 444 bp, Oryza sativa
atgagctttgcaatccgcagctctgagcctgaattctggttcttgatcccgtcggaagaggcagcagtagcagtcgcagcacatcggctggtggtgatggatcagaggagaagcggatcagcttatcgtcctaagcggacacatatggcggcggcggaggacgagcaccggcggccggggacgtcgagccgccgccgggtggcgccgacgccgacgacgcagacgcagacgcagacggcgcccggctacttcaccgtcgagctggtgatggcgttcgtctgcgtgaccgcgtcgctcgtgctgctgccgctcgtcctgccgccgttgccgccgccgccgtcgctgctgctggtggtgccggtgtgcctgctcgccgtcctggtggccatggcgttcgtcccgctcgacgcgcagagcaacgtcgtcggctcgtcttgcttgtag

SEQ ID NO: 49 - DNA, 537 bp, Oryza sativa
atgtacttgttgagcccaagaaatggcgacgaggaggacgaacaggaggaaatccaggagctgatcagcgacgacgagccgcccaatctcaagttggcatcctgcgccactgcagccagcagcagcagcagcagcggcagcgacatggagaagggaagaggtaaagcctgcggcggcgggagtacggcgccgccgccgccgccgccgtcgtcgtcaggtaaatccggcggcggcggcggcagcaatatcagggaggcggcggctagcggcggcggcggcggcgtgtggggcaagtacttctcggtggagtcgctgctcctgctggtgtgcgtgacggcgtcgctggtgatcctcccgctcgtgctgccgccgctgcccccgccgccgtcgatgctgatgctggtgccggtggcgatgctggtgctgctgctggcgctggcgttcatgccgacgacgacgtcgtcgtcgtcgtccgccggcggcggcggcggcggcggccgcaatggggcgacgacgggacatgctccctacttgtag

SEQ ID NO: 50 - DNA, 384 bp, Oryza sativa
atgcgaggagtcatcttgctgcgttacgaggaggacgccatggccgggcacaggtccacggcggcggcgacgggagggagattgtacggacaggtgggagtgaagcggagagtggtggaggagacggcggcggcggtggaagtaggcggaggaggaggagggtacttgggggtggaggcggcggtgctgctcggggtggtgacggcgacgctgctggtgctgccgctgctgctgccgccgctgccgccgccgccgccgatgctgctgctcgtgcccgtcgccatcttcgccgtgctcctcctcctcgtcctgctgccctccgacgccaagtccatcgccgccgctggccgaccctcttcttcctcctcctcctcctacctgtag

SEQ ID NO: 51 - DNA, 318 bp, Oryza sativa
atgcaagaagaagcggcgtcgtcgtcgtcgtcgtcggcgtcgccggtgatggacgggggcaaggcgatggcggtgctgctggcggtggcggccgcggtgctgctgctgctcccgctcgtgctgccgtcgctgctgctgctcctccccgtggtgctgctcctgctggtggtttccctcgccttcttccccgcggccggcagcgacggcgtcgtcgccgccgccgcggtcgccggcacctaccagccgccgccgcctccgcctgctcggtcgtcaccgccgccgtcgtcgtcgtcgtcatcgtcgtcgcggcagctgtga

SEQ ID NO: 52 - DNA, 318 bp, Oryza sativa
atggaaggtgtaggtgctaggcagaggaggaaccctctgatacccagaccaaacggttcaaagaggcatctgcagcatcagcatcagccaaatgctgccgagaagaagaccgccgcgacatcgaattacttcagtatcgaggcgttcctcgtgctcgtcttcctcaccatgtcattgctcatacttccattggtgcttcccccattgcctccgccgccatcgctgctgctgctgctgccagtctgcctgctcatcctgctggttgtgctggccttcatgccaacggatgtgcggagcatggcttcctcttacttgtaa

SEQ ID NO: 53 - DNA, 363 bp, Oryza sativa
atggaggaacagatgttcagagagcagcaaatgcagagaggtggaaggcatcatcagcatcacaccacaagggaacaagaacaacagcagaagcagcagcagcggcggcggctgatgaacaatgcgaccaacggcggcggcggcgacggcggcagcaggtgctacttcagcacggaggccatcctggtgctggcatgcgtcaccgtgtcgctgctggtgctgccgctcatcctgccgccgctgccgccgccgccgacgctgctgctgctgctgccggtgtgcttgctggcgctcctggtggtgctggccttcatgcccactgacatgaggaccatggcctcttcctactttttttgtttgtga

SEQ ID NO: 54 - DNA, 291 bp, Glycine max
atgatgatggtgcatcctcgtgatcaagtaggtggagagacacacaagaatttggtggagccaaacgtggcagcttctaagaaagctagaaattgtgcatgcatggtaagttactcggtgttgattttggctcttctcactttgtccattttgttgctacctttggtgttacctcctctgccgccaccacccttgttgcttctctttgttccagttttcatcttggtggttctctttttcttggccttttcaccctccacactacccaacatggctgttcttacatcatga

SEQ ID NO: 55 - DNA, 291 bp, Glycine max
atgatgatggtgcatcctcgtgatcaagtaggtggagacacacacaagaatttggtggcgccaaacgtggcagcttctaagaaagctagaaattgtgcatgcatggtaagttattcggtgttgattttggctcttctgactttgttcattttgttgctgcctttggtgttgcctcctctgccggcaccacccttgttgcttctctttgttcctgttttcctcttggtggttctctttttcttggccttttcaccttccacactacccaacatggctgttcttacatcatga

SEQ ID NO: 56 - DNA, 312 bp, Glycine max
atggcgcgttgttttggtttaggttccgttctggttctggcggcgctcgcggcgtcgatggtggttctgccgctgatgctgccgccgctcccgccgccgccactagttcttctcttcttccccgtcgggatcatggcggcgctcatgttgctcgcgttctcgccatcagatcaaaacggcgtcgtttacgcgtcgacgtagcgaaggtggtgggaaaccggatcagccggtgccacattttggggtttcttgaaggttccgatgggattgcttcgtttcatgtttttttttttttttaagttacggtgttaa

SEQ ID NO: 57 - DNA, 201 bp, Glycine max
atggcgcgttgtttcggcttaggttccgttctggttctggcggcgctcgcggcgtcgatggtggtgctgccgttgatgctcccgccgctcccgccgccgccgctggtttttttttttttccccgtcgggatcatggcggcgctcatgttgcttgtgttctcgccgtcggatcaaaacggcgtcgtttacgccaccacgtaa

SEQ ID NO: 58 - DNA, 273 bp, Glycine max
atgagctcttggttgattcactacaacaagagattcataataagcatctcattagcgtttatgctaaggctttttgggtttaaatcaaccatgttcatggtggtgctgaccatagcaatcttggttctaccactgatgctaccacctctacctccaccaccaatgattcttatgttggtgcctcttgtgataatgctgcttctggtgaaattggctttgtattccaaacatggccctgcagatgtcatttatcagtgtaattttacttggtag

SEQ ID NO: 59 - DNA, 393 bp, Arabidopsis thaliana
atgattcgagaaatctcaaacttacaaaaagatattataaacattcaagacagttattcgaacaaccgagtcatggacgtcggaagaaacaaccggaaaaacatgagctttcgaagttcgccggagaaaagcaagcaagagttacggcggagtttctcggcgcagaaaaggatgatgatcccggcgaattatttcagtttagagtctctgttcctattggttggtctaacggcatctctgttaatacttccgttagttttgccgccgttacctccgcctccgtttatgctgctattggttcccattgggattatggttttactcgtcgttcttgccttcatgccttcttctcattctaatgctaatacagatgtaacttgcaatttcatgtaa

SEQ ID NO: 60 - DNA, 408 bp, Arabidopsis thaliana
atgattcgtgagttctccagtctacaaaacgacatcataaacattcaagaacattattctctcaacaacaacatggacgtgagaggagatcataaccggaaaaacacgagttttcgtggttcagctccagctccgattatggggaagcaagaattgtttcggacattgtcgtcgcagaacagtccaaggaggctaatatcagcgagttacttcagtttagaatcaatggttgtgcttgttggtctcacagcatctctcttgatcttaccgttgattcttccaccattgcctcctcctccttttatgctgcttttgattcctattgggattatggttttgcttatggttcttgctttcatgccttcttctaattccaaacatgtttcttcttcttccacttttatgtaa

SEQ ID NO: 61 - DNA, 267 bp, Arabidopsis thaliana
atgagggttcatgatcaacggctgagatttgatgtcacacccaagccgatgggtttgaacggaagttctttgatcacggcaagatccgtcgcacttcttctctttctctctctgcttcttctgattctgccaccgttcctgccgccgcttccaccgcctccggcgacactcctcctccttcctctactcctcatgattctcctcattttcttggctttttctccttctaatgagcccagcctcgccgttgaacctctcgacccctga

SEQ ID NO: 62 - DNA, 441 bp, Sorghum bicolor
atgagcaccggccggccggaggacatccagcagctaatcaacagtgccactagtagccccaaccgcactagtccatccgcctcgcccagcgacatggagagcggcggcggaagcgcgtcctcgccgcgcgcttcgacgtccgaccggcgcctgcagagggccgcccacagtcacagggaggagtgggagcctgctgctgctgctagcggcgatggcggcacgggtagcctctggtccaggtacttctcgctcccggtcctcctgctcgtcggcgtcaccgcgtcgctggtgatcctcccgctcgtgctccccccgctaccgccgccgccgtcgatgctgatgctggtcccggtggcaatgctggtcttgctgctcgtgctggcgttcatgccgacgtcgagcgtccgcgctgggacggggacggggccgacctacatgtag

SEQ ID NO: 63 - DNA, 267 bp, Sorghum bicolor
aatgccgtgaagcggcacctgcagcagcggcagcaggaggcggatttccacgacaagaaggtcatcgcgtccacctacttcagcatcggcgcgttcctggtgctcgcctgcctcaccttctcgctgctcatcctgcctctggtgctgccgccgctgccgccgccgccgtcgctgctgctgtggctgccggtctgcctgctcgtcctgctggttgtgctggccttcatgccgacagatgtgcgcagcatggcctcctcttacttgtaa

SEQ ID NO: 64 - DNA, 324 bp, Sorghum bicolor
atggcaagccgatctagcgcgctggaaggagggggggcagcaatacagcggaggaataatgccgtgaagcggcacctgcagcagcggcagcaggaggcggatttccacgacaagaaggtcatcgcgtccacctacttcagcatcggcgcgttcctggtgctcgcctgcctcaccttctcgctgctcatcctgccgctggtgctgccgccgctgccgccgccgccgtcgctgctgctgtggctgccggtctgcctgctcgtcctgctggttgtgctggccttcatgccgacagatgtgcgcagcgtggcggcctcttacttgtaa

SEQ ID NO: 65 - DNA, 177 bp, Sorghum bicolor
atgatgttgctggtggcgacagtgatcctcctgtgcctgccattggtgctgccaccacttccgccaccaccgctgttccttctcttcgtccctgtggtgatgatgctcctgctcttctccctggttctcttcccgtctcaccactgtgcatgctcttctccaaccttcactcagtaa

SEQ ID NO: 66 - DNA, 447 bp, Sorghum bicolor
atgagctttgtggccggcagctctgaggctgatcaactctggttcttgatcccgtcggaacaagcacgagctcacgcggtacagcctcatcatccgttggccatggaccggaggtcgtcggcgaggaggagaggcgatcctcaccctcaccgccggggcgcaatgcacggtgccgccgagcagcagaagcagcagcagcagcgcggccggccgcagggaacgcgggcggcgccgcccgtgccgccgggctacttcacggcggagctggtgctggcgttcctgttcgtggccgtgtcgctggcgttcctcccgctggtcctgccgccgctgtcgccgccgccgttcctgctgctgctggtgcccgtgggactgctggccgtgctcctcgcgctcgcgttcgtgccgctcgacgcgcacagccacctcgtcgtcggctcctcccgctga

SEQ ID NO: 67 - DNA, 363 bp, Sorghum bicolor
atggcggaggagaggaagcaggcgggctcccgctggcccgccggaggcagcggcggcgggcgaatgcgcgacgccgagggtggcagtggcaagatgcggggccggcaggcaacaaaggcaaggcccgtagtactggcgccgccgggccaggggtacttcacggcggggctggcggcgctgttcctctgcctcaccgcgctgctggtgttcctgccgctcgtgctgcccccgctgccgccgccgccgtatcttctgctgctcgtgccggtgggcctcatggccgtactgctggctctggtggcgctcgtgccgtccgacggccgggccgccaccgccgccgtcgcgtcgtcgtgcgtgtgctga

SEQ ID NO: 68 - DNA, 306 bp, Sorghum bicolor
atgcggcgggcggtgccgcaggaggaagccgtggcggcggcgacgacgacgaccatggacgggggcaaggtggtggcgctgctggccacggcggccgcgctgctgctgctcctcccgctggcgctgcccccgctgccgccgccgcccacgcagctgctgttcgtccccgtcgtcatgctgctgctcgtggcgtccctcgccttctgccccaccgccgcgagcagcggcggcggcggcaagagcaagctcgccgacgccgaccacgggtcgtcgtttcggactactggatcaccgcacctgcgctga

SEQ ID NO: 69 - DNA, 321 bp, Sorghum bicolor
atgccgtcgccgtcgcagacatcgccgccggtcgggaggcggactgctcatggcggctggcacaagcacgatgacccaagcacgccgaggggcttctgcaccaagtacttctccgtggagtcgtgcctcctgctcgccctcgtcgccgtgctgctgctggtgctcccgctcgtcctgccgccgctcccgccgccgccgttggcggtgctgctcgtgccggtcgcaatgttggcggtgctgctggtgctggcgctcatgccggtggcggcggcggcggcgggtgcccggaacgaggtcgtggacccggcgtcgtacttgtag

SEQ ID NO: 70 - DNA, 219 bp, Sorghum bicolor
atggaacgaagcatggtgacgatgctgctcctcgcgacggcggccgtggtgcttctgctgctcccgctgctgctcccttcttccctgccgccaccgccgtcgctgctgctggtcgtccctgtcgtgctgctgctctcgctgctttccctcgctttccttcccacccgcgacgacgatgacgctattgctatctacggatcactccgatccgtgcagtga

SEQ ID NO: 71 - DNA, 2436 bp, Zea mays
aacaattcttgctacatgacataaaaataataaatggtcgactgacttgttacactgacagaaataacatccaagtctcccaatccaattccccttaatgaagttagcttttgttagcaaggcccattttctatgagccacataaagaacttgatttttggtggttttttttctttacaaatgtgtttaaaatgtaaaccggtttcatttctagtaaggtattgtatacagatttgacagaaaatcttccatttttcctagatttccagataatgctttctggactgtcattaagatggaacgacaccacttgttcctaaatcttatcccacatatatatacttgaggtcatccactaataattagatttttggcacacttactttcttctggttacataattaataagggcctgtttagataccaggagctaaagaaaaagtggttaaagtttagtcaatttagggggttaaagatctaaaccggaagaatgagtgactaaaataataaaagtgtacccttttagtcacttttagctcctaagaagaagctaacctttaatcagtttgcttttaacccctggatccaaacaagtcctaaatcaccggtataactaggcacaatgcctcatcagacagccaactgccaatcccaaattctactttgttgtccatttctaattttaatgcctctgcctgcacgtatactatttttgtttttgattaagtcataccacataggagaatcactcaatttattagtaattgtattgtatgaactgaatcatttggcgtatatttggctttctttaagcacaggcacactgctaccaaaagcattaggcgcttaagcatcacccttgtggctggcacgagaaccatttgattcacgacagatttagcgcttgtttgaagtgttggctaaaactagcaatctacagggaagaacaccatacattagtactccgtcgggacacgccaccacatgccgctgaaatatatccgcaacgattctcccagctgcttatagctcagaagcaagagccaaggccggcagctaaccacgactcgtctaatcatccctggaccatagcgattaataaattgattaagctagtacatcgcccctagatttccggcagaattaagaaaaccgcgggcagcagagccgatgccgatggcaacaaagaagaaggggctgttggtactgcagccgcagtctataaagataaaaattgtagaagtagtaggaagcttagccggagctggcatggcaggctgctgctgagtgagcagcggtgggggcctcctggctggcgcgtggaaaaacccgagcaaatggcagcgtgaagcacgtccgagactgaggtcaggcgtcgggcgggggttgcggccaggggagacgaatgaacccctgcccccgcctggatcccatcgcaaaagccccctccccctctccgccttcgcgcatattatattcgcgcaccatcgcagcaacttgcacgggcgccgatgactagttgcgccagatgcactgcatctgctcggcgtggtgcctccaacgtccaacccctcttcctcttgtctctcgtctacctctcttctgcccctctgcgtccgtgtctccatcgtcgtcgctgcgtgaggttgacgacgaccagtcacaggacctgttcgttcctcatgcgacccagctagctaaaactggcatgcatggacatgctacgctgctgcgtcaatccatctcaccaggtacgctgctgtacatgctgctacgagcctacgatcgatagcagtctgtgccttcctttgctcgatgccgatgtttatctgcatgtgatcgtattcgtatgcacggccctccgccctctcaagctgagtgctttttggtgggcccatcgtcctatatacgctcatcagttcactgacgacgatataacgactgttggggttcagaaactacatattgtggtgctcgcccgatctctttcttgtatattcttcttattattagtctctctctctctgaaagaacaaggaactagatgtcttgttttgtgcctcctactatacctttgcgtgtttttcttgcttttgtccatggcttttcaccggtctgctgggtgaagtaatttacacgcatgtcttacgcacgcgctccttcagttgtccgcatatctgatcataacatcgcttcattcatgtgctgacgagatatttttcgccgccgagactgcagtgctagctagctagatctggcctgattcgccgatcgagcggtggtgagacggagtgcttcagctcaaagactgctagtggtaggctggtagctagctgtgtgcctgtgtgcagtgtgcactgccactgcatgcgcggcgccttggacttaagacggcagcacacgcacgcgaggaggcgtcggctgaagcgagcgctc

SEQ ID NO: 72 - DNA, 709 bp, Paspalum notatum
ggtggcggcggcggcttaattactagatctggcctgattcggcggtcggccgcgagcatgcagtcagagatgatgagatcaagtgcttcagctgcaagactctagctgcatgcgtgccgtcgacttcaagaaggcgcacgcaaagggcaagggcatcgatcgcgaggtcgatcgactggaccgatcgaacgctccgatccgatcgtcctcaatcctcatgttcttgagcccgccggggaggaatgagcagccggaggacatccagcaactgatcaacagcagcgccgctggtcccagcctgaatccacctgccgcgcccagcagccccagcagcgacagcgacatgatggtggagagcggcggcggcggcggacgcgcgtcctcgtctcctgcttgttgcacttcatcgacgtccggccagagggcccacagggaggaggaggagctttgcagccatggcggctggtggtcgtccagcagcagcaggtacttgtcgctgccgctcctgctgctcgtcggcgtcaccgcgctgctgctgatcctcccgctcgtcctgccgccgctgcccccgccgccgtcgatgctcatgctggtccccgtggcaatgctcgtgttgctgctcgtgctggcgttcatgccgacgacgtccggcggccgtgctggcaccgcggggccgacctacatgtagataatcacatctttttttttt

SEQ ID NO: 73 - protein, 192 aa, Paspalum notatum
MRAVDFKKAHAKGKGIDREVDRLDRSNAPIRSSSILMFLSPPGRNEQPEDIQQLINSSAAGPSLNPPAAPSSPSSDSDMMVESGGGGGRASSSPACCTSSTSGQRAHREEEELCSHGGWWSSSSSRYLSLPLLLLVGVTALLLILPLVLPPLPPPPSMLMLVPVAMLVLLLVLAFMPTTSGGRAGTAGPTYM

SEQ ID NO: 74 - DNA, 656 bp, Paspalum notatum
aagcactgccaggctcgagatcagagatactattggcgcagatcttcttagctgctgcagacctgcagtcagagcgccaaggatccatttctgaaaccgagtaggcaccgagctcagatagcatcgctcgtcggaataattgagcaagctcattgctgcgcgttggatgctggcagctgagttgctcttgctcatgggcatggcaatggagatggagcagatcgccagggatcagcggcgccccaggcggcagcagggcaggcggcaggccgtggtggtcgtctcgagcaggcacggcggcgccgcggcgaagggacagcgtcagaacgtgccgccgtcgcccaggtcgacggctgctgccgggctcagcgcggaggcgttcctcgtgctcgcctgcgtcgccgtctcgctcatcgtgctgccgctggtcctgccgccgctgccgcccccgccgccgttgctgctgctggtgcccgtgtgcctgctcctcctcctggcagcgctcgccaccttcgtgccgtccgatgtgaagaccatggcgtcctcctacatgtaaatgtttctagttgtagtcttgtaatataaaaattttatttaatctgttccggctatttctgtatgtttttggcataaaatgagtgcaacgaaatgaaatt

SEQ ID NO: 75 - protein, 129 aa, Paspalum notatum
MLAAELLLLMGMAMEMEQIARDQRRPRRQQGRRQAVVVVSSRHGGAAAKGQRQNVPPSPRSTAAAGLSAEAFLVLACVAVSLIVLPLVLPPLPPPPPLLLLVPVCLLLLLAALATFVPSDVKTMASSYM

SEQ ID NO: 76 - DNA, 343 bp, Paspalum notatum
caaaatggcaagccgatccggcgcgatggaagaaggcggcggcgggacgaggcagaggaggagcccagcaagtgctgcaaagcggcactttcagcagcagaggcagcaggaggcggatttctacgacaggaaggtgatggcgtccacctacttcagcatcggcgccttcctcgtgctcgcctgcctcaccgtctcgctgctcatcctgccgctggtgctgccgccgctgccgccgccgccgtcgctgctgctctggctgcccgtctgcctgctcctcctgctcatcgtgctcgccttcatgcccaccgatgtgcggagcatggcctcctcctacctgtaaata

SEQ ID NO: 77 - protein, 111 aa, Paspalum notatum
MASRSGAMEEGGGGTRQRRSPASAAKRHFQQQRQQEADFYDRKVMASTYFSIGAFLVLACLTVSLLILPLVLPPLPPPPSLLLWLPVCLLLLLIVLAFMPTDVRSMASSYL

SEQ ID NO: 78 - DNA, 476 bp, Paspalum notatum
cgcgccctcaattttgtggacatatatatataggacgacgataccttctctccctctcgcctacctcttctcagtctcagcctctcaggacgcgcgcatgcacaaacccacacccgcacatacctaacctgacgacgcttcttgtaggcagctatggaaggaagcatggtgatgctgctcgtcgccacagcggccgtggtgcttctgctgcttcctctgctgctccctcccctgccgccgccgccgtcgctgctgctgatcgtccccgtcgtcctactgctctcgctgctttccctggctttcgtccccagtacaaagctccatggatcgtcgactgatcgttttatgcagcgagacgcagcacaggcgtacgtgcgtgtttaactctgcgcctctctacggcgctgctacttaattacaatgaggcgagacgcatgcgtgcacacaagagactgatgcagctagcgtacgtcgtc

SEQ ID NO: 79 - protein, 76 aa, Paspalum notatum
MEGSMVMLLVATAAVVLLLLPLLLPPLPPPPSLLLIVPVVLLLSLLSLAFVPSTKLHGSSTDRFMQRDAAQAYVRV

SEQ ID NO: 80 - DNA, 879 bp, Poaceae sp
ggggttctcgcctcaccggcgtctggtgagcgccgccgaaggacatccagcaaccatccgaccggttggagaggcaaaccaacgccacagcctttgaagcggcggcacgagaatggagtcgccgcagggcgggagggcagctcacctcgacggccggctcaagtacgacgacccgagcacgccgaggggcttctgcgccaagtacttctccgtgaagtcgtgcctcctactcgccgtcgtcaccgtgctgctgctggtgctcccgctcgtcctgccgccgctcccgccgccgccgatgctgctgctgctcgtgccggtggcgatgctggccatgctgctgctactggcgctcacgccgccgcgctgccgacagaacgaagctgtggacgcgacatctaattacctgtaggttccagttttgagcaagttaaagggcatacaccatctcggtgatcagcaatgcacttaattttgtttgtgtatataaatctatttttatgttgtcttactccagttttttattttcgaacatgggcatgacgttaattggacagttggacttatgctgacatggacctggtttggacatctaattaagcatccacagacagttcttgtcgtgaaatagcgggggaacaacagcggagatggatgctaccgctacgtagcgtactactagatgacatgccagcaaaaccattcgctgggggtggtaaaatagacggtataaaggattcgagggtgttcactgtccaatattgcagtaaatgcagttcaagattactttagaccctacggatagttttttaggtacaaattgagttttaccaaattcatatgacaacgccctggatgcttttacaagaaagcgttgtcacg

SEQ ID NO: 81 - protein, 98 aa, Poaceae sp
MESPQGGRAAHLDGRLKYDDPSTPRGFCAKYFSVKSCLLLAVVTVLLLVLPLVLPPLPPPPMLLLLVPVAMLAMLLLLALTPPRCRQNEAVDATSNYL

SEQ ID NO: 82 - DNA, 937 bp, Poaceae sp
ccgtcaattcctttaagtttcagccttgcccgcccccacgccgccaaaagcggtgacaccgcccaccgcctcctcctccccgttcgttcggctaccgttgcgccgcagccgcaggtcgccattaagtcctctgcgctttttgcccggcgtgcgtcctcttggttcttcttgcacaactctgctgctgctgctgctggtttcctccagctctgttttttcttccgttcttttcctcctgtcagtcctggtcaaatccatcctccattcctcctggatcgcttttggaaattcccgcgggctgccgttcttggtttgtgttcttggtggtattaatctggagatccaatcacttggggacgagatcaagaccgccaagaaacagaacgggcaaaagccggcatcgccaagcttaacatcagaggttgccgcgtcgctcatggcgatggagttggagacggaccacctcgccaggcggcagcagagcaggaggcaggccaagggccagcagcagcagcagcagcagcgccagaacgcgccgtcgcccaagcctcctgctccggcggcggcggcagcgggcgggctgagcgccgaggcgttcctggcgctggcgtgcgtggccgtgtcgctcgtcgtgctgccgctcgtcctgccgccgctgccgcccccgccgccgctgctgctgctcgtgcccgtctgcctgctcctgctcctcgccgcgctcgccaccttcgtgccgtcggcggatgtcaggaccatggcgtcctcctacttgtaactagctcactagttgtttagtgagagtttatgcataattaattctttcttttttgttcccgccggcccttttcttctgtgtatatggataaaatgagtgtaatgatgaaatggaaatcttgttcttttgtttgtttgtatttttctttttctgaaacagaga

SEQ ID NO: 83 - protein, 112 aa, Poaceae sp
MAMELETDHLARRQQSRRQAKGQQQQQQQRQNAPSPKPPAPAAAAAGGLSAEAFLALACVAVSLVVLPLVLPPLPPPPPLLLLVPVCLLLLLAALATFVPSADVRTMASSYL

SEQ ID NO: 84 - protein, 15 aa, Poaceae sp
LVVLPLVLPPLPPPP

SEQ ID NO: 85 - protein, 15 aa, Artificial sequence
  conserved region
LLVLPLVLPPLPPPP

SEQ ID NO: 86 - protein, 15 aa, Artificial sequence
  Conserved region with variables
LXXLPLXLPPLXXPP

SEQ ID NO: 87 - protein, 72 aa, Zea mays
MVATTTMAGGKVAALLATAAALLLLLPLALPPLPPPPTQLLFVPVVLLLLVASLAFCPAATSSPSPMHAADH

SEQ ID NO: 88 - protein, 9 aa, Zea mays
LPPLPPPPS

SEQ ID NO: 89 - protein, 56 aa, Zea mays
SGYLSLPALLLVGVTASLVILPLVLPPLPPPPSMLMLVPVAMLLLLLVLAFMPTSS

SEQ ID NO: 90 - protein, 23 aa, Zea mays
SGYLSLPALLLVGVTASLVILPL

SEQ ID NO: 91 - protein, 25 aa, Zea mays
PSMLMLVPVAMLLLLLVLAFMPTSS

SEQ ID NO: 92 - protein, 22 aa, Artificial sequence
  primer
DYKDDDDKVKLYPYDVPDYAAA

SEQ ID NO: 93 - protein, 8 aa, Artificial sequence
  linker
GGGSGGGS

SEQ ID NO: 94 - DNA, 19 bp, Artificial sequence
  primer
gtctgcaccatcgtcaacc

SEQ ID NO: 95 - DNA, 21 bp, Artificial sequence
  primer
gaagtccagctgccagaaacc

SEQ ID NO: 96 - protein, 8 aa, Zea mays
  misc_feature (4)..(4): Xaa can be any naturally occurring amino acid
PPLXPPPX

SEQ ID NO: 97 - protein, 15 aa, Sorghum bicolor
LLVLPLVLPPLPPPP

SEQ ID NO: 98 - protein, 15 aa, Sorghum bicolor
LLILPLVLPPLPPPP

SEQ ID NO: 99 - protein, 15 aa, Sorghum bicolor
LVILPLVLPPLPPPP

SEQ ID NO: 100 - protein, 15 aa, Sorghum bicolor
LLVLPLLLPPLPPPP

SEQ ID NO: 101 - protein, 15 aa, Sorghum bicolor
LVFLPLVLPPLPPPP

SEQ ID NO: 102 - protein, 8 aa, Artificial sequence
  consensus
PPLDPPPD

SEQ ID NO: 103 - DNA, 204 bp, Arabidopsis thaliana
atgtttgtgattggagtggtgatggtgctattggcggttcttccagccgttctgccgccgcttccgccgccgccgatgatattgatgggaattccggtggtgctgatgctaatgcttatttacttagccatttattatccacctcatcaagctcattttctctcttcatcttcctttgacactacttctaggcatgtaatgtga

SEQ ID NO: 104 - protein, 67 aa, Arabidopsis thaliana
MFVIGVVMVLLAVLPAVLPPLPPPPMILMGIPVVLMLMLIYLAIYYPPHQAHFLSSSSFDTTSRHVM